(19) United States
(12) Patent Application Publication
(10) Pub. No.: US 2009/0263407 A1
(43) Pub. Date: Oct. 22, 2009

(54) Cationic Lipids and Uses Thereof

(75) Inventors: Prasad A. Dande, Evanston, IL (US); Todd M. Hansen, Grayslake, IL (US); Robert D. Hubbard, Lindenhurst, IL (US); Carol K. Wada, Evanston, IL (US); Lu Tian, Evanston, IL (US); Xiaobin Zhao, Potomac, MD (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, Abbott Park, IL (US)

(21) Appl. No.: 12/425,254

(22) Filed: Apr. 16, 2009

Related U.S. Application Data
(60) Provisional application No. 61/045,350, filed on Apr. 16, 2008.
(60) Provisional application No. 61/170,015, filed on Apr. 16, 2009.

(51) Int. Cl. A61K 47/18; C07D 207/08; C07D 317/26; C07D 317/28; C07C 229/34; C07C 217/48; C07C 217/28; A61K 47/22; A61K 31/7088; A61K 31/7105; A61K 39/00; A61K 38/02

(52) U.S. Cl. 424/184.1; 548/568; 549/453; 549/451; 554/103; 554/111; 564/391; 564/504; 514/785; 514/788; 514/44 R; 514/44 A; 514/2

(57) ABSTRACT

Cationic lipids, cationic lipid based drug delivery systems, ways to make them and methods of treating diseases using them are disclosed.

This application claims priority to U.S. Provisional Application Ser. No. 61/045,350 filed Apr. 16, 2008.

### FIELD OF THE INVENTION

This invention pertains to cationic lipids, cationic lipid based drug delivery systems, ways to make them, and methods of treating diseases using them.

### BACKGROUND OF THE INVENTION

Through the development of novel delivery formulations, research is now able to focus more on improving efficacy on the therapeutic and clinical efficacious of therapeutic agents such as nucleic acids, RNA, antisense oligonucleotide, a DNA, a plasmid, a ribosomal RNA (rRNA), a micro RNA (miRNA), transfer RNA (tRNA), a small inhibitory RNA (siRNA), and small nuclear RNA (snRNA). Such novel delivery formulations will need, for example, to allow for appropriate internalization of the therapeutic agent into the cell, agents sufficient absorption from the site of administration, distribution to various tissues, sufficient residence time and concentration at the sites of action to elicit effective biologic response, in addition to also maintaining it's stability, and size. To this end, many efforts have been made to develop liposome or cationic polymer complexes with polyethylene glycol (PEG) or other neutral or targeting moieties. Ogris et al., Gene Ther. 6, 595-605 (1999).

However, many of the complexes to date have not been found to successfully deliver therapeutic agents. As such, there is a clear need in the art to develop a novel liposomal delivery system that can enhance therapeutic agent efficacy.

### SUMMARY OF THE INVENTION

One embodiment of this invention, therefore pertains to a cationic lipid or mixtures thereof, having Formula (I)

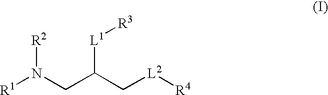

wherein

R¹ and R² are independently H, cycloalkyl, cycloalkenyl or R⁵; or

R¹ and R², taken together with the atoms to which they are attached, are heterocycloalkyl or heteroaryl;

L¹ is O, OC(O) or (O)CO;

L² is O, OC(O) or (O)CO;

one of R³ and R⁴ is H, and the other is C₁₄-C₂₀-alkenyl or C₁₄-C₂₀-alkyl; or

R³ and R⁴ are independently C₁₄-C₂₀-alkenyl or C₁₄-C₂₀-alkyl; or

R³ and R⁴ combine to form CR²⁰R²¹, wherein R²⁰ is H and R²¹ is C₁₄-C₂₀-alkenyl or C₁₄-C₂₀-alkyl; or R²⁰ and R²¹ are the same or are different and are C₁₄-C₂₀-alkenyl, C₁₄-C₂₀-alkyl, or (CH₂O)—C₁₄-C₂₀ alkenyl;

R⁵ is alkyl, which is unsubstituted or substituted with one or more R⁶, OR⁶, SR⁶, S(O)R⁶, SO₂R⁶, C(O)R⁶, CO(O)R⁶, OC(O)R⁶, OC(O)OR⁶, NH₂, NHR⁶, N(R⁶)₂, NHC(O)R⁶, NR⁶C(O)R⁶, NHS(O)₂R⁶, N R⁶S(O)₂R⁶, NHC(O)OR⁶, NR⁶C(O)OR⁶, NHC(O)NH₂, NHC(O)NHR⁶, NHC(O)N(R⁶)₂, NR⁶C(O)NHR⁶, NR⁶C(O)N(R⁶)₂, C(O)NH₂, C(O)NHR⁶, C(O)N(R⁶)₂, C(O)NHOH, C(O)NHOR⁶, C(O)NHSO₂R⁶, C(O)NR⁶SO₂R⁶, SO₂NH₂, SO₂NHR⁶, SO₂N(R⁶)₂, C(O)H, C(O)OH, C(N)NH₂, C(N)NHR⁶, C(N)N(R⁶)₂, CNOH, CNOCH₃, OH, (O), CN, N₃, NO₂, CF₃, CF₂CF₃, OCF₃, OCF₂CF₃, F, Cl, Br or I;

R⁶ is R⁷, R⁸, R⁹ or R¹⁰;

R⁷ is phenyl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R⁸ is heteroaryl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R⁹ is cycloalkyl, cycloalkenyl, heterocycloalkyl or heterocycloalkenyl, each of which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹⁰ is alkyl, alkenyl or alkynyl;

wherein each foregoing cyclic moiety is independently unsubstituted or substituted with one or more R¹¹, OR¹¹, SR¹¹, S(O)R¹¹, SO₂R¹¹, C(O)R¹¹, CO(O)R¹¹, OC(O)R¹¹, OC(O)OR¹¹, NH₂, NHR¹¹, N(R¹¹)₂, NHC(O)R¹¹, NR¹¹C(O)R¹¹, NHS(O)₂R¹¹, N, R¹¹S(O)₂R¹¹, NHC(O)OR¹¹, NR¹¹C(O)OR¹¹, NHC(O)NH₂, NHC(O)NHR¹¹, NHC(O)N(R¹¹)₂, NR¹¹C(O)NHR¹¹, NR¹¹C(O)N(R¹¹), C(O)NH₂, C(O)NHR¹¹, C(O)N(R¹¹)₂, C(O)NHOH, C(O)NHOR¹¹, C(O)NHSO₂R¹¹, C(O)NR¹¹SO₂R¹¹,SO₂NH₂, SO₂NHR¹¹,SO₂N(R¹¹)₂, C(O)H, C(O)OH, C(N)NH₂, C(N)NHR¹¹, C(N)N(R¹¹)₂, CNOH, CNOCH₃, OH, (O), CN, N₃, NO₂, CF₃, CF₂CF₃, OCF₃, OCF₂CF₃, F, Cl, Br or I;

R¹¹ is R¹², R¹³, R¹⁴,or R¹⁵;

R¹² is phenyl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹³ is heteroaryl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹⁴ is cycloalkyl, cycloalkenyl, heterocycloalkyl or heterocycloalkenyl, each of which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹⁵ is alkyl, alkenyl or alkynyl, each of which is unsubstituted or substituted with one or two of independently selected R¹⁶, OR¹⁶, SR¹⁶, S(O)₂R¹⁶, C(O)OH, NH₂, NHR¹⁶N(R¹⁶)₂, C(O)R¹⁶, C(O)NH, C(O)NHR¹⁶, C(O)N(R¹⁶)₂, NHC(O)R¹⁶, NR¹⁶C(O)R¹⁶, NHC(O)OR¹⁶NR¹⁶C(O)OR¹⁶,OH, F, Cl, Br or I;

R¹⁶ is alkyl, alkenyl, alkynyl, or R¹⁷;

R¹⁷ is phenyl, heteroaryl, cycloalkyl, cycloalkenyl, heterocycloalkyl or heterocycloalkenyl, each of which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

wherein R¹², R¹³, R¹⁴, and R¹⁷ are independently unsubstituted or substituted with one or more R¹⁸,OR¹⁸, SR¹⁸S(O)R¹⁸, SO₂R¹⁸, C(O)R¹⁸, CO(O)R¹⁸, OC(O)R¹⁸, OC(O)OR¹⁸, NH₂, NHR¹⁸, N(R¹⁸)₂, NHC(O)R¹⁸, NR¹⁸C(O)R¹⁸, NHS(O)₂R¹⁸, NR¹⁸S(O)₂R¹⁸, NHC(O)OR¹⁸, NR¹⁸C(O)OR¹⁸, NHC(O)NH₂, NHC(O)NHR¹⁸, NHC(O)N(R¹⁸)₂, NR¹⁸C(O)NHR¹⁸, NR¹⁸C(O)N(R¹⁸)₂, C(O)NH₂, C(O)NHR¹⁸, C(O)N(R¹⁸)₂, C(O)NHOH, C(O)NHOR¹⁸, C(O)NHSO₂R¹⁸, C(O)NR¹⁸SO₂R¹⁸,SO₂NH₂, SO₂NHR¹⁸, SO₂N(R¹⁸)₂, C(O)H, C(O)OH, C(N)NH₂, C(N)NHR¹⁸, C(N)N(R¹⁸)₂, CNOH, CNOCH₃, OH, (O), CN, N₃, NO₂, CF₃, CF₂CF₃, OCF₃,OCF₂CF₃, F, Cl, Br or I; and

R¹⁸ is alkyl, alkenyl, alkynyl, phenyl, heteroaryl, cycloalkyl, cycloalkenyl, heterocycloalkyl or heterocycloalkenyl.

A further embodiment pertains to Cationic-Based Lipid Encapsulation Systems (CaBLES) comprising one or more non-cationic lipids, one or more polyethylene glycol (PEG)-lipid conjugates and one or more cationic lipids having Formula I.

Another embodiment of the present invention is cationic lipids of the present invention (i.e., cationic lipids of Formula I) which can be used in the preparation of either empty liposomes or used to deliver any product (e.g., therapeutic agents including nucleic acids, diagnostic agents, labels or other compounds) to a cell tissue, including cells and tissues in mammals.

In still a further embodiment, Lipid-Based Particles of the present invention are defined as CaBLES which further comprise one or more therapeutic agent(s). Such Lipid-Based Particles can be used to deliver any of a variety of therapeutic agent(s), preferably said therapeutic agent is a nucleic acid encoded with a product of interest, including but not limited to, RNA, antisense oligonucleotide, a DNA, a plasmid, a ribosomal RNA (rRNA), a micro RNA (miRNA), transfer RNA (tRNA), a small inhibitory RNA (siRNA), small nuclear RNA (snRNA), antigens, fragments thereof, proteins, peptides, vaccines and small-molecules or mixtures thereof.

A further embodiment pertains to pharmaceutical compositions comprising a Lipid-Based Particle and a pharmaceutically acceptable carrier.

A further embodiment pertains to a method of treating cancer in a mammal comprising administering thereto a therapeutically acceptable amount of a Lipid-Based Particle. Yet another embodiment pertains to a method of decreasing tumor volume in a mammal comprising administering thereto a therapeutically acceptable amount of a Lipid-Based Particle.

A further embodiment pertains to a method of making CaBLES or Lipid-Based

Particles, comprising: (a) mixing the cationic lipid(s), the non-cationic lipid(s) and the PEG-lipid conjugate(s); (b) adding the mixture of step (a) to one or more therapeutic agents; and

(c) separating and purifying resulting suspension of step (b).

### DESCRIPTION OF THE DRAWINGS

FIGS. 1-40. In vitro and in vivo transfection activity of selected cationic lipids that were formulated as disclosed herein.

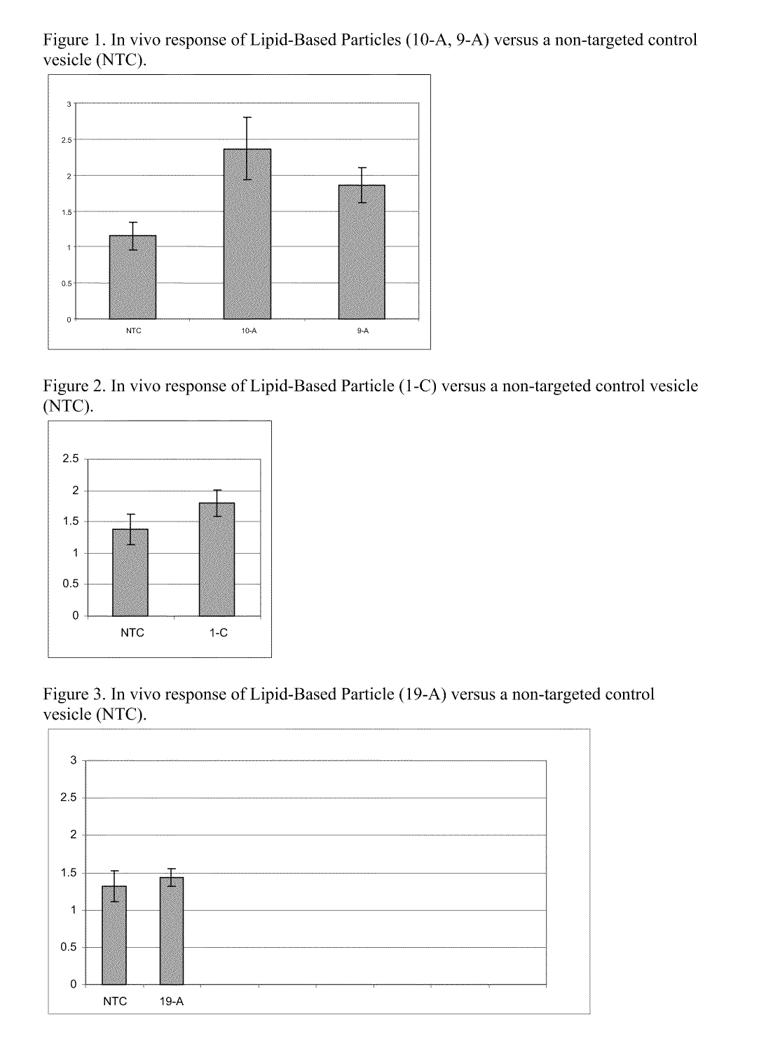

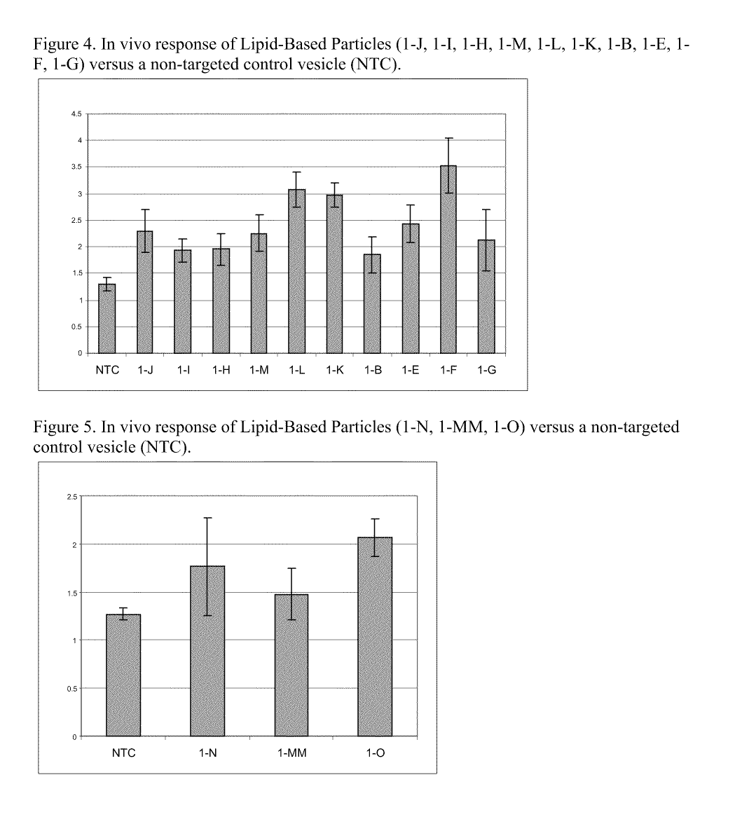

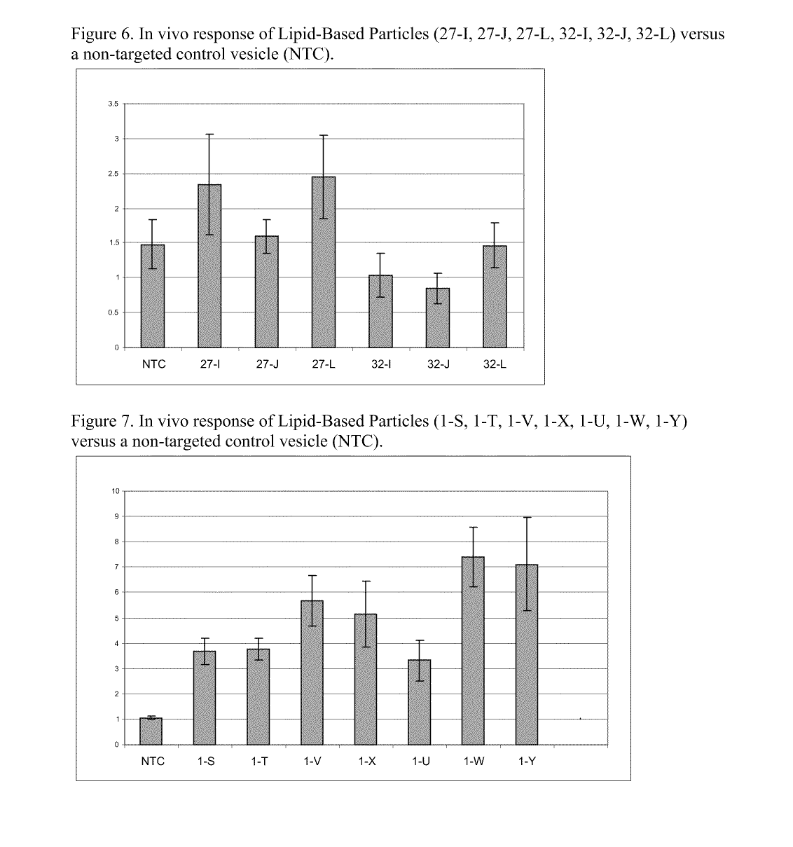

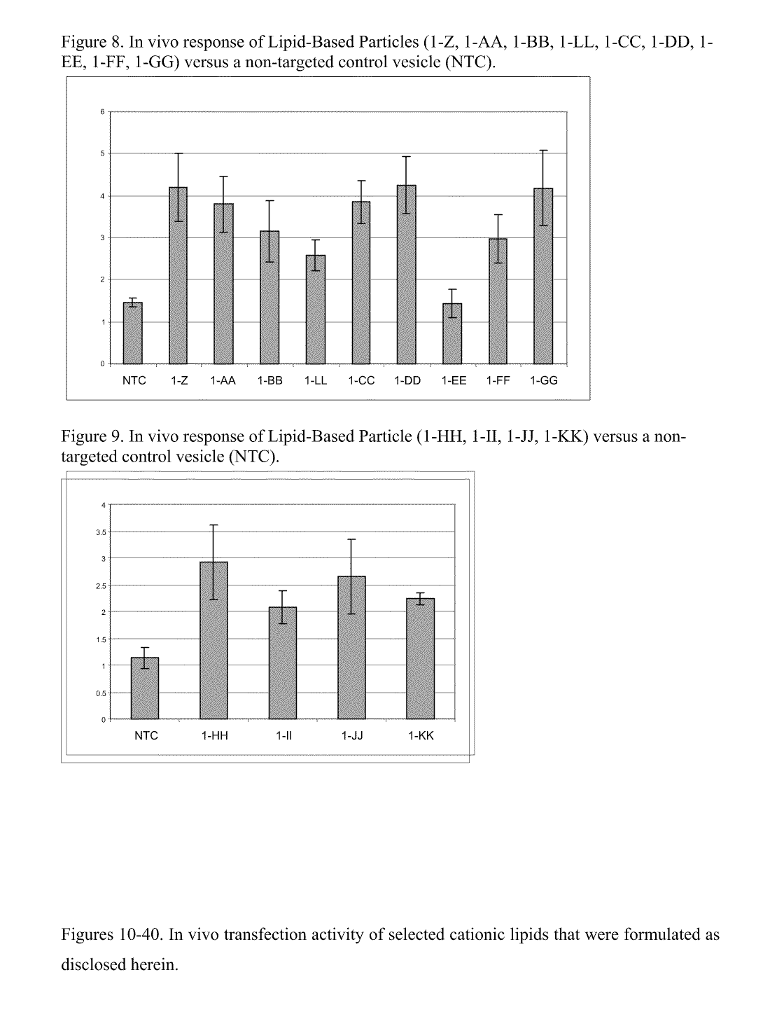

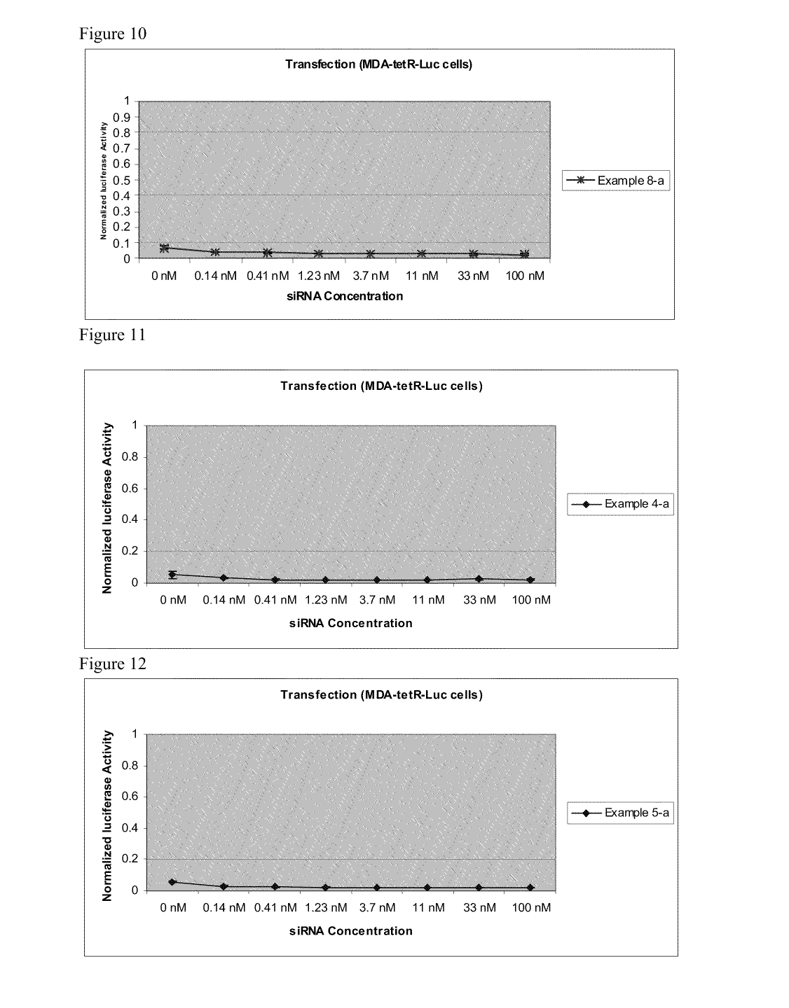

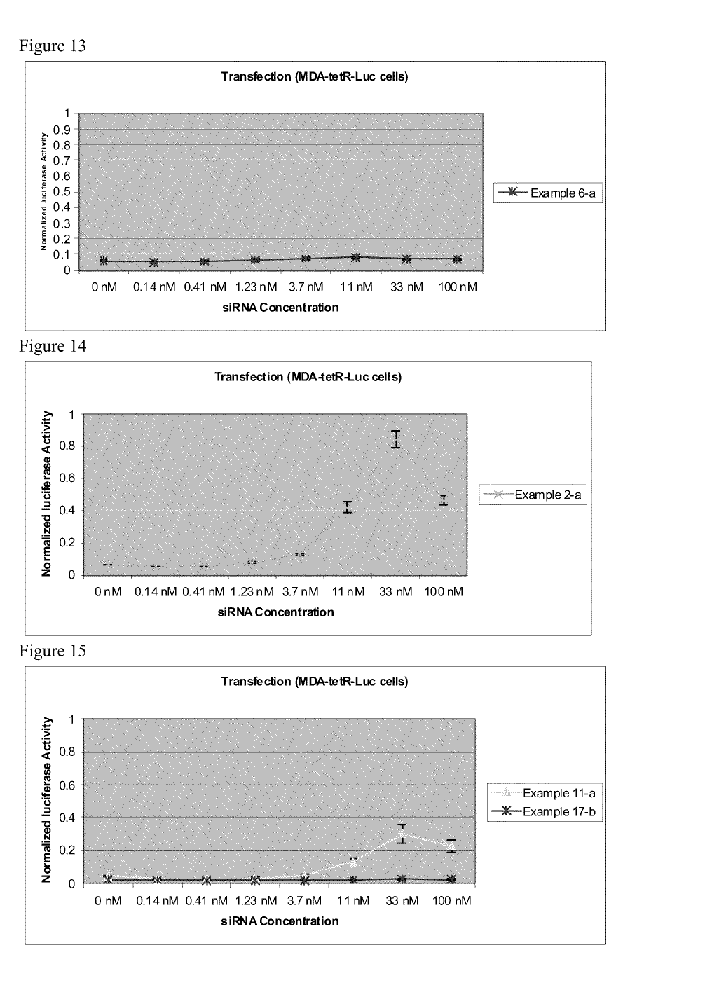

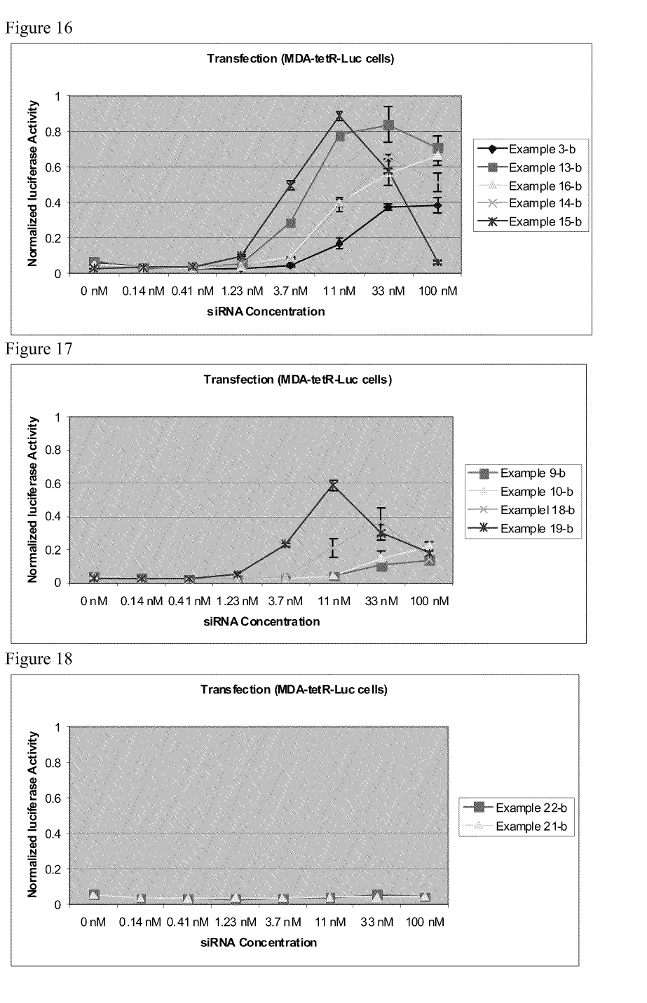

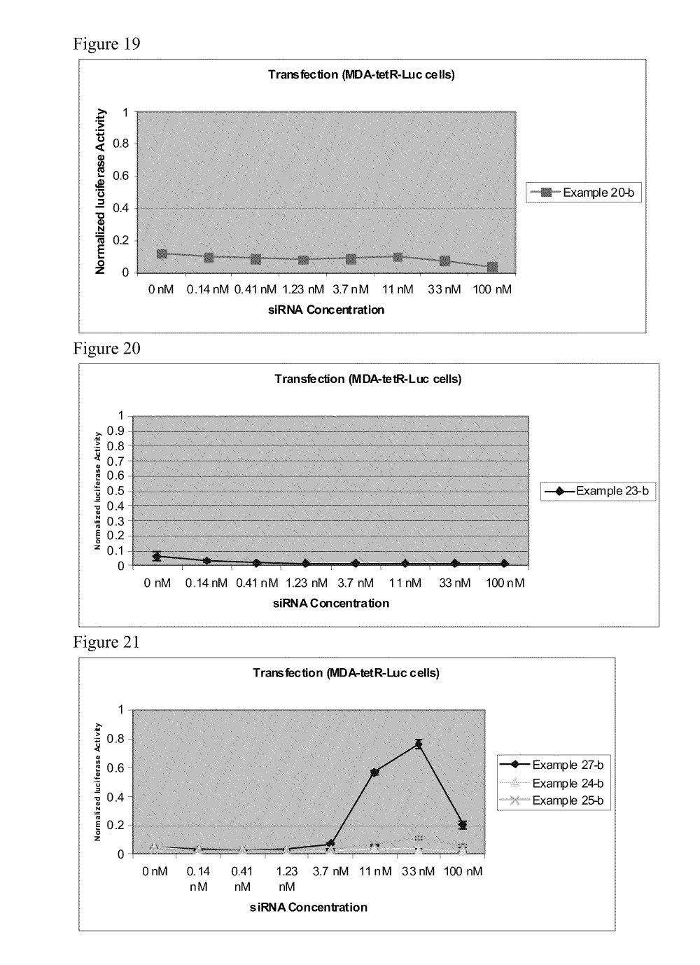

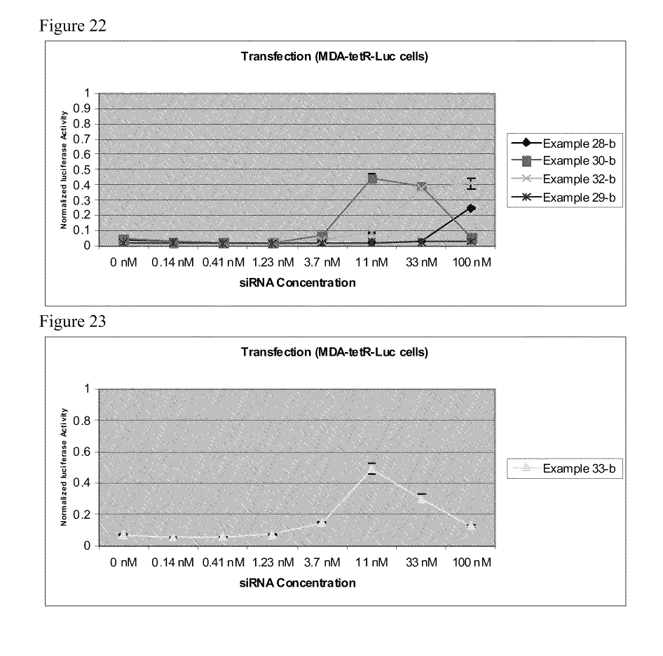

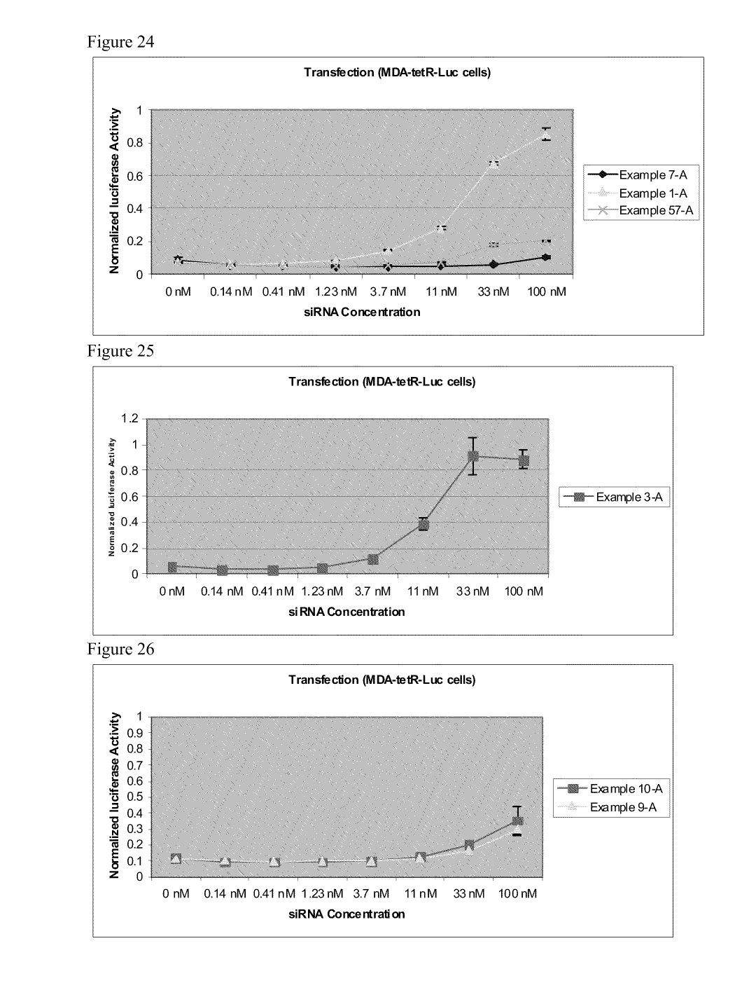

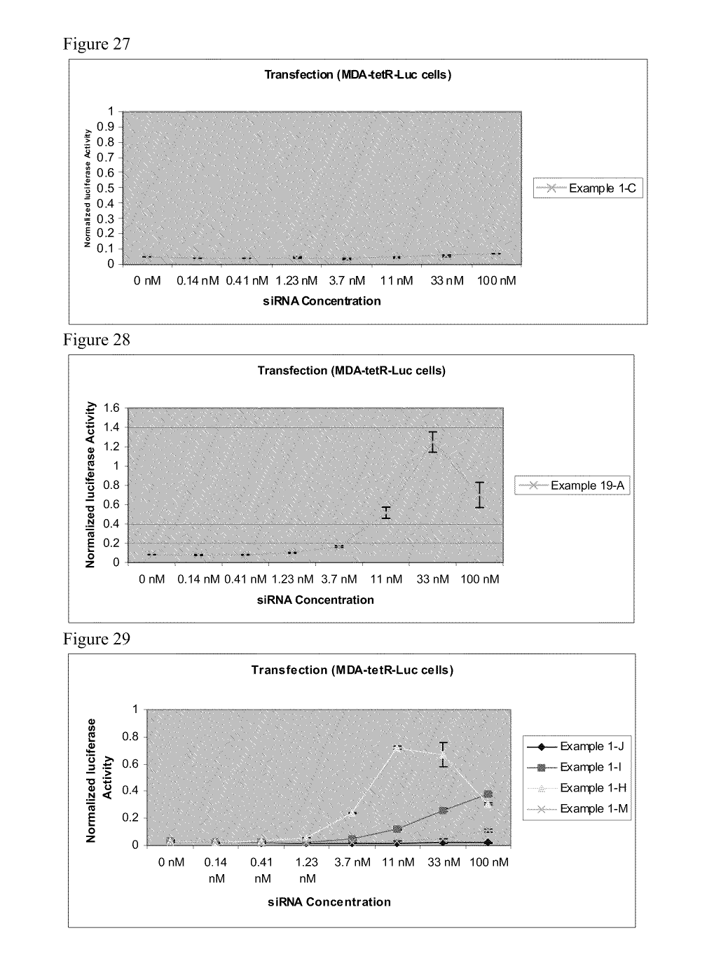

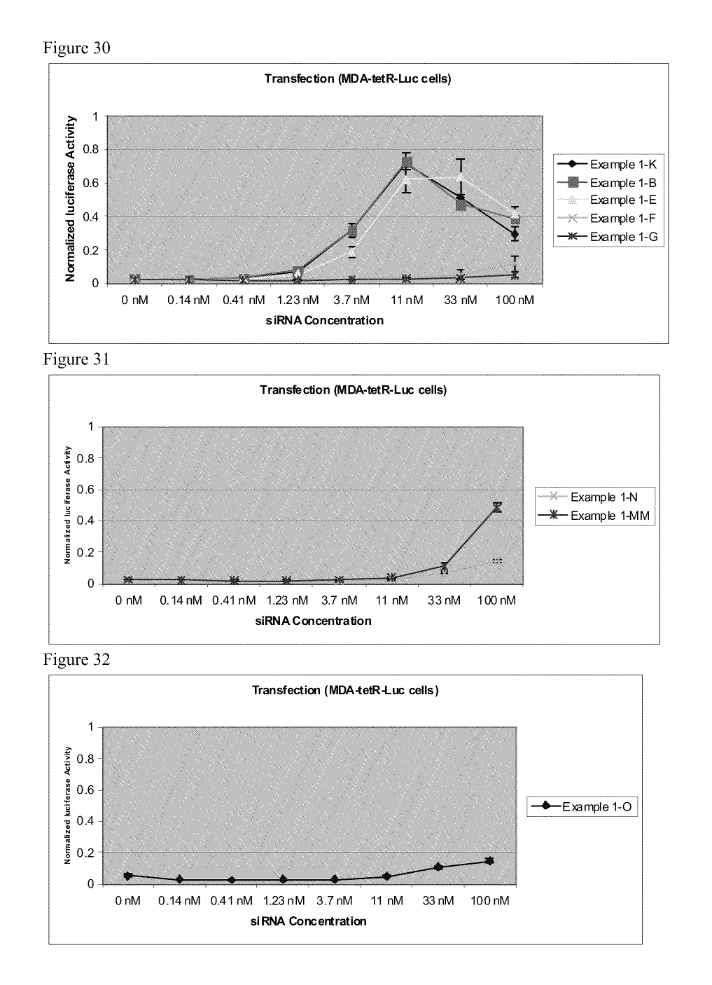

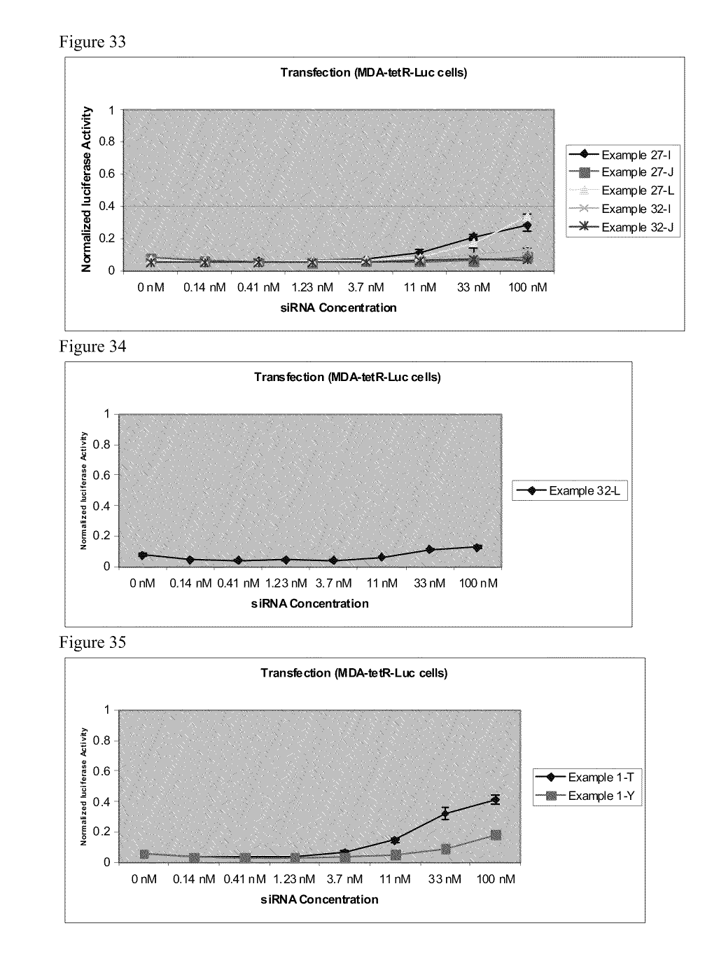

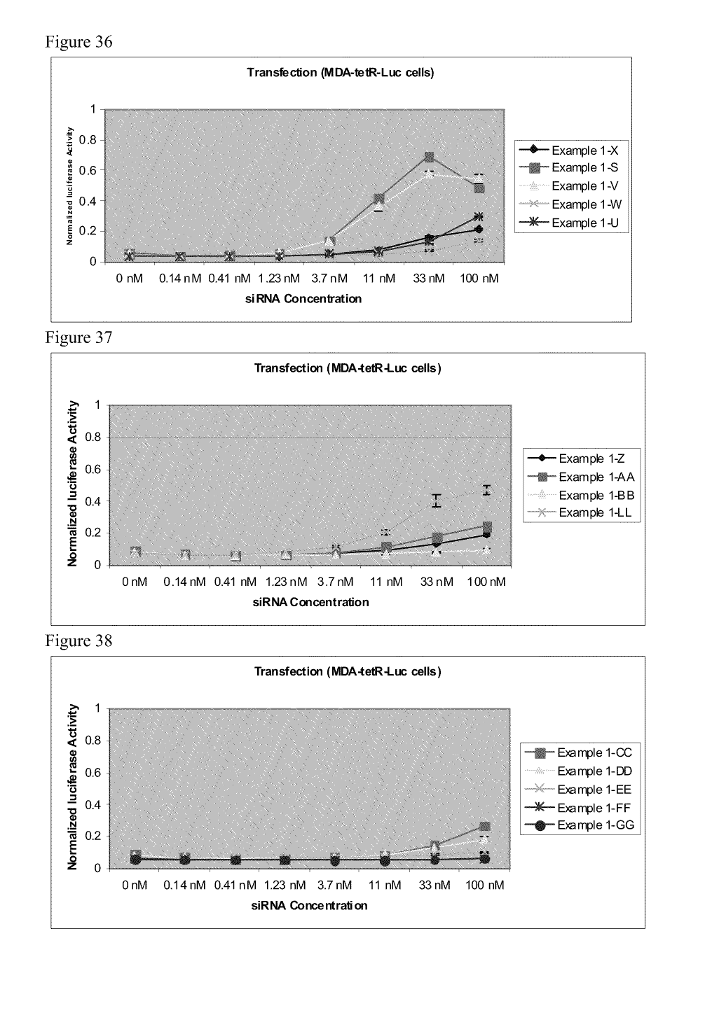

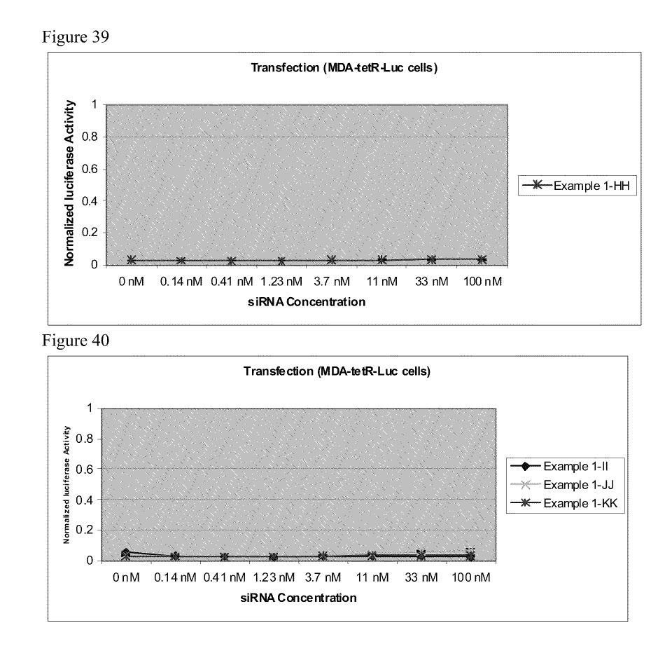

### DETAILED DESCRIPTION OF THE INVENTION

This invention pertains to in vitro and in vivo delivery of therapeutic agents. In particular, the invention pertains to compositions that allow for delivery of nucleic acids, including but not limited to RNA, antisense oligonucleotide, a DNA, a plasmid, a ribosomal RNA (rRNA), a micro RNA (miRNA), transfer RNA (tRNA), a small inhibitory RNA (siRNA), small nuclear RNA(snRNA), antigens, fragments thereof, proteins, peptides, and small molecules.

Variable moieties of compounds herein are represented by identifiers (capital letters with numerical and/or alphabetical superscripts) and may be specifically embodied.

It is also meant to be understood that a specific embodiment of a variable moiety may be the same or different as another specific embodiment having the same identifier and that asymmetric divalent moieties are drawn from left to right.

As used in the specification and the appended claims, unless specified to the contrary, the following terms have the meaning indicated:

The term “alkenyl,” as used herein, means monovalent, straight or branched chain hydrocarbon moieties having one or more than one carbon-carbon double bonds, such as C₂₋alkenyl, C₃₋alkenyl, C₄₋alkenyl, C₅₋alkenyl, C₆₋alkenyl and the like.

The term “C₁-C₆-alkylene,” as used herein, means divalent, saturated, straight or branched chain hydrocarbon moieties bonds, such as C₁-alkylene, C₂₋alkylene, C₃₋alkylene, C₄₋alkylene, C₅₋alkylene and C₆₋alkylene.

The terms “alkyl,” as used herein, means monovalent, straight or branched chain hydrocarbon moieties such as C₁-alkyl, C₂₋alkyl, C₃₋alkyl, C₄₋alkyl, C₅₋alkyl and C₆₋alkyl.

The term “alkynyl,” as used herein, means monovalent, straight or branched chain hydrocarbon moieties having one or more than one carbon-carbon triple bonds, such as C₂₋alkynyl, C₃₋alkynyl, C₄₋alkynyl, C₅₋alkynyl, C₆₋alkynyl and the like.

The term “C₁-C₈-alkyl” as used herein, means C₁-alkyl, C₂₋alkyl, C₃₋alkyl, C₄₋alkyl, C₅₋alkyl, C₆₋alkyl, C₇₋alkyl and C₈₋alkyl.

The term “C₁₄-C₂₀-alkenyl,” as used herein, means C₁₄-alkenyl,” C₁₅-alkenyl,” C₁₆-alkenyl,” C₁₇— alkenyl,” C₁₈-alkenyl,” C₁₉-alkenyl” and C₂₀-alkenyl.”

The term “C₁₄-C₂₀-alkyl,” as used herein, means C₁₄-alkyl,” C₁₅-alkyl,” C₁₆-alkyl,” C₁₇-alkyl,” C₁₈-alkyl,” C₁₉-alkyl” and C₂₀-alkyl.”

The term “cycloalkane,” as used herein, means saturated cyclic or bicyclic hydrocarbon moieties, such as C₃-cycloalkane, C₄-cycloalkane, C₅-cycloalkane, C₆-cycloalkane and the like.

The term “cycloalkyl,” as used herein, means monovalent, saturated cyclic and bicyclic hydrocarbon moieties, such as C₃-cycloalkyl, C₄-cycloalkyl, C₅-cycloalkyl, C₆-cycloalkyl and the like.

The term “cycloalkene,” as used herein, means cyclic and bicyclic hydrocarbon moieties having one or more than one carbon-carbon double bonds, such as C₅-cycloalkene, C₆-cycloalkene and the like.

The term “cycloalkenyl,” as used herein, means monovalent, cyclic hydrocarbon moieties having one or more than one carbon-carbon double bonds, such as C₄-cycloalkenyl, C₅*cycloalkenyl, C₆-cycloalkenyl and the like.

The term “heteroarene,” as used herein, means a five-membered or six-membered aromatic ring having at least one carbon atom and one or more than one independently selected nitrogen, oxygen or sulfur atom. The heteroarenes of this invention are connected through any adjacent atoms in the ring, provided that proper valences are maintained. Examples of heteroarenes include, but are not limited to furan, imidazole, isothiazole, isoxazole, oxadiazole, oxazole, pyrazine, pyrazole, pyridazine, pyridine, pyrimidine, pyrrole, thiazole, thiadiazole thiophene, tetrazine, tetrazole, triazine, triazole and the like.

The term “heteroaryl,” as used herein, means a monovalent five-membered or six-membered aromatic ring having at least one carbon atom and one or more than one independently selected nitrogen, oxygen or sulfur atom. The heteroaryls of this invention are connected through any carbon atom or any nitrogen atom in the ring, provided that proper valences are maintained. Examples of heteroaryls include, but are not limited to, furanyl, imidazolyl, isothiazolyl, isoxazolyl, oxadiazolyl, oxazolyl, pyrazinyl, pyrazolyl, pyridazinyl, pyridinyl, pyrimidinyl, pyrrolyl, tetrazolyl, thiadiazolyl, thiazolyl, thienyl, triazinyl, triazolyl and the like.

The term “heterocycloalkane,” as used herein, means cycloalkane having one or two or three CH₂ moieties replaced with independently selected O, S, S(O), SO₂ or NH and one or two CH moieties unreplaced or replaced with N and also means cycloalkane having one or two or three CH₂ moieties unreplaced or replaced with independently selected O, S, S(O), SO₂ or NH and one or two CH moieties replaced with N.

The term “heterocycloalkene,” as used herein, means cycloalkene having one or two or three CH₂ moieties replaced with independently selected O, S, S(O), SO₂ or NH and one or two CH moieties unreplaced or replaced with N and also means cycloalkene having one or two or three CH₂ moieties unreplaced or replaced with independently selected O, S, S(O), SO₂ or NH and one or two CH moieties replaced with N.

The term “heterocycloalkyl,” as used herein, means cycloalkyl having one or two or three CH₂ moieties replaced with independently selected O, S, S(O), SO₂ or NH and one or two CH moieties unreplaced or replaced with N and also means cycloalkyl having one or two or three CH₂ moieties unreplaced or replaced with independently selected O, S, S(O), SO₂ or NH and one or two CH moieties replaced with N.

The term “heterocycloalkenyl,” as used herein, means cycloalkenyl having one or two or three CH₂ moieties replaced with independently selected O, S, S(O), SO₂ or NH and one or two CH moieties unreplaced or replaced with N and also means cycloalkenyl having one or two or three CH₂ moieties unreplaced or replaced with independently selected O, S, S(O), SO₂ or NH and one or two CH moieties replaced with N.

The term “cyclic moiety,” as used herein, means benzene, cycloalkane, cycloalkyl, cycloalkene, cycloalkenyl, heteroarene, heteroaryl, heterocycloalkane, heterocycloalkyl, heterocycloalkene, heterocycloalkenyl and phenyl.

The term “DSPC,” as used herein, means 1,2-distearoyl-sn-glycero-3-phosphocholine.

The term, “Chol,” as used herein, means cholesterol.

The term, “PEG-Chol,” as used herein, means poly(oxy-1,2-ethanediyl)-2000-α-(3β)-cholest-5-en-3-yl-omega-hydroxy.

The term, “Pal-PEG-Cera,” as used herein, means N-palmitoyl-sphingosine-1-[succinyl(methoxypolyethylene glycol)-2000].

The term, “PEG-DMPE,” as used herein, means N-(carbonyl-methoxypolyethyleneglycol-2000)-1,2-dimyristoyl-sn-glycero-3-phosphoethanolamine.

The term, “PEG-DPPE,” as used herein, means N-(carbonyl-methoxypolyethyleneglycol-2000)-1,2-dipalmitoyl-sn-glycero-3-phosphoethanolamine.

The term, “PEG-DSPE,” as used herein, means N-(carbonyl-methoxypolyethyleneglycol-2000)-1,2-distearoyl-sn-glycero-3-phosphoethanolamine.

The term, “PEG-DMG,” as used herein, means 1,2-dimyristoyl-sn-glycerol-methoxypolyethyleneglycol-2000.

The term, “PEG-DPG,” as used herein, means 1,2-dipalmitoyl-sn-glycerol-methoxypolyethyleneglycol-2000.

The term, “PEG-DSG,” as used herein, means 1,2-distearoyl-sn-glycerol-methoxypolyethyleneglycol-2000.

The term “MALDI,” as used herein, means matrix assisted laser desorption ionization.

The term, “particle,” as used herein, means a small object that behaves as a whole unit in terms of its transport and properties.

The term, “nanoparticle,” as used herein, means any particle having a diameter of less than 1000 nanometers. In some embodiments, nanoparticles have a diameter of 500 or less. In some embodiments, nanoparticles have a diameter of 200 or less.

The term “nucleic acid” or “polynucleotide” refers to a polymer containing at least two deoxyribonucleotides or ribonucleotides in either single- or double-stranded form. Nucleic acids include nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Unless specifically limited, the terms encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)). “Nucleotides” contain a sugar deoxyribose (DNA) or ribose (RNA), a base, and a phosphate group. Nucleotides are linked together through the phosphate groups. Nucleotides include chemically modified nucleotides as described in, e.g., WO 03/74654. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. DNA may be in the form of antisense, plasmid DNA, parts of a plasmid DNA, pre-condensed DNA, product of a polymerase chain reaction (PCR), vectors (P1, PAC, BAC, YAC, artificial chromosomes), expression cassettes, chimeric sequences, chromosomal DNA, or derivatives of these groups. The term nucleic acid is used interchangeably with gene, plasmid, cDNA, mRNA, and an interfering RNA molecule (e.g. a synthesized siRNA or an siRNA expressed from a plasmid).

The term, “siRNA,” as used herein, means a small inhibitory RNA, and molecules having endogenous RNA bases or chemically modified nucleotides. The modifications shall not abolish cellular activity, but rather impart increased stability and/or increased cellular potency. Examples of chemical modifications include phosphorothioate groups, 2′-deoxynucleotide, 2′-OCH₃-containing ribonucleotides, 2′-F-ribonucleotides, 2′-methoxyethyl ribonucleotides or a combination thereof.

The term, “SPC,” as used herein, means soybean phosphatidylcholine.

The term “small molecule,” as used herein, means antibiotics, antineoplastics, antiinflammatories, anitivirals, immunomodulators and agents that act upon the respiratory system, the cardiovascular system, the central nervous system or a metabolic pathway involved with dyslipidemia, diabetes or Syndrome X.

### Compounds

Compounds of this invention may contain asymmetrically substituted carbon atoms in the R or S configuration, wherein the terms “R” and “S” are as defined in Pure Appl. Chem. (1976) 45, 13-10. Compounds having asymmetrically substituted carbon atoms with equal amounts of R and S configurations are racemic at those atoms. Atoms having excess of one configuration over the other are assigned the configuration in excess, preferably an excess of about 85%-90%, more preferably an excess of about 95%-99%, and still more preferably an excess greater than about 99%. Accordingly, this invention is meant to embrace racemic mixtures and relative and absolute diastereoisomers and the compounds thereof.

Compounds of this invention may also contain carbon-carbon double bonds or carbon-nitrogen double bonds in the E or Z configuration, wherein the term “E” represents higher order substituents on opposite sides of the carbon-carbon or carbon-nitrogen double bond and the term “Z” represents higher order substituents on the same side of the carbon-carbon or carbon-nitrogen double bond as determined by the Cahn-Ingold-Prelog Priority Rules. The compounds of this invention may also exist as a mixture of “E” and “Z” isomers.

One embodiment of this invention, therefore pertains to a cationic lipid or mixtures thereof, having Formula (I)

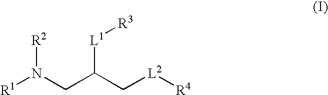

wherein

R¹ and R² are independently H, cycloalkyl, cycloalkenyl or R⁵; or

R¹ and R², taken together with the atoms to which they are attached, are heterocycloalkyl or heteroaryl;

L¹ is O, OC(O) or (O)CO;

L² is O, OC(O) or (O)CO;

one of R³ and R⁴ is H, and the other is C₁₄-C₂₀-alkenyl or C₁₄-C₂₀-alkyl; or

R³ and R⁴ are independently C₁₄-C₂₀-alkenyl or C₁₄-C₂₀-alkyl; or

R³ and R⁴ combine to form CR²⁰R²¹, wherein R²⁰ is H and R²¹ is C₁₄-C₂₀-alkenyl or C₁₄-C₂₀-alkyl; or R²⁰ and R²¹ are the same or are different and are C₁₄-C₂₀-alkenyl, C₁₄-C₂₀-alkyl, or (CH₂O)—C₁₄-C₂₀ alkenyl;

R⁵ is alkyl, which is unsubstituted or substituted with one or more R⁶, OR⁶, SR⁶, S(O)R⁶, SO₂R⁶, C(O)R⁶, CO(O)R⁶, OC(O)R⁶, OC(O)OR⁶, NH₂, NHR⁶, N(R⁶)₂, NHC(O)R⁶, NR⁶C(O)R⁶, NHS(O)₂R⁶, N R⁶S(O)₂R⁶, NHC(O)OR⁶, NR⁶C(O)OR⁶, NHC(O)NH₂, NHC(O)NHR⁶, NHC(O)N(R⁶)₂, NR⁶C(O)NHR⁶, NR⁶C(O)N(R⁶)₂, C(O)NH₂, C(O)NHR⁶, C(O)N(R⁶)₂, C(O)NHOH, C(O)NHOR⁶, C(O)NHSO₂R⁶, C(O)NR⁶SO₂R⁶, SO₂NH₂, SO₂NHR⁶, SO₂N(R⁶)₂, C(O)H, C(O)OH, C(N)NH₂, C(N)NHR⁶, C(N)N(R⁶)₂, CNOH, CNOCH₃, OH, (O), CN, N₃, NO₂, CF₃, CF₂CF₃, OCF₃, OCF₂CF₃, F, Cl, Br or I;

R⁶ is R⁷, R⁸.,R⁹, or R¹⁰

R⁷ is phenyl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R⁸ is heteroaryl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R⁹ is cycloalkyl, cycloalkenyl, heterocycloalkyl or heterocycloalkenyl, each of which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹⁰ is alkyl, alkenyl or alkynyl;

wherein each foregoing cyclic moiety is independently unsubstituted or substituted with one or more R¹¹,OR¹¹, SR¹¹S(O)R¹¹, SO₂R¹¹, C(O)R¹¹, CO(O)R¹¹, OC(O)R¹¹, OC(O)OR¹¹, NH, NHR¹¹, N(R¹¹)₂, NHC(O)R¹¹, NR¹¹C(O)R¹¹, NHS(O)₂R¹¹, N R¹¹S(O)₂R¹¹, NHC(O)OR¹¹, NR¹¹C(O)OR¹¹, NHC(O)NH₂, NHC(O)NHR¹¹, NHC(O)N(R¹¹)₂, NR¹¹C(O)NHR¹¹, NR¹¹C(O)N(R¹¹)₂, C(O)NH₂, C(O)NHR¹¹, C(O)N(R¹¹)₂, C(O)NHOH, C(O)NHOR¹¹,C(O)NHSO₂R¹¹,C(O)NR¹¹SO₂R¹¹, SO₂NH₂, SO₂NHR¹¹,SO₂N(R¹¹)₂, C(O)H, C(O)OH, C(N)NH₂, C(N)NHR¹¹, C(N)N(R¹¹)₂, CNOH, CNOCH₃, OH, (O), CN, N₃, NO₂, CF₃, CF₂CF₃, OCF₃, OCF₂CF₃, F, Cl, Br or I;

R¹¹ is R¹², R¹³, R¹⁴, or R¹⁵;

R¹² is phenyl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹³ is heteroaryl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹⁴ is cycloalkyl, cycloalkenyl, heterocycloalkyl or heterocycloalkenyl, each of which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹⁵ is alkyl, alkenyl or alkynyl, each of which is unsubstituted or substituted with one or two of independently selected R¹⁶,OR¹⁶,SR¹⁶,S(O)₂R¹⁶,C(O)OH, NH₂, NHR¹⁶N(R¹⁶)₂, C(O)R¹⁶,C(O)NH₂, C(O)NHR¹⁶,C(O)N(R¹⁶)₂, NHC(O)R¹⁶, NR¹⁶C(O)R¹⁶, NHC(O)OR¹⁶NR¹⁶C(O)OR¹⁶,OH, F, Cl, Br or I;

R¹⁶ is alkyl, alkenyl, alkynyl, or R¹⁷

R¹⁷ is phenyl, heteroaryl, cycloalkyl, cycloalkenyl, heterocycloalkyl or heterocycloalkenyl, each of which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

wherein R¹², R¹³, R¹⁴, and R¹⁷ are independently unsubstituted or substituted with one or more R¹⁸, OR¹⁸, SR¹⁸, S(O)R¹⁸, SO₂R¹⁸, C(O)R¹⁸, CO(O)R¹⁸, OC(O)R¹⁸, OC(O)OR¹⁸, NH₂, NHR¹⁸, N(R¹⁸)₂, NHC(O)R¹⁸, NR¹⁸C(O)R¹⁸, NHS(O)₂R¹⁸, NR¹⁸S(O)₂R¹⁸, NHC(O)OR¹⁸, NR¹⁸C(O)OR¹⁸, NHC(O)NH₂, NHC(O)NHR¹⁸, NHC(O)N(R¹⁸)₂, NR¹⁸C(O)NHR¹⁸, NR¹⁸C(O)N(R¹⁸)₂, C(O)NH₂, C(O)NHR¹⁸OC(O)N(R¹⁸)₂, C(O)NHOH, C(O)NHOR¹⁸, C(O)NHSO₂R¹⁸, C(O)NR¹⁸SO₂R¹⁸, SO₂NH₂, SO₂NHR¹⁸, SO₂N(R¹⁸)₂, C(O)H, C(O)OH, C(N)NH₂, C(N)NHR¹⁸, C(N)N(R¹⁸)₂, CNOH, CNOCH₃, OH, (O), CN, N₃, NO₂, CF₃, CF₂CF₃, OCF₃, OCF₂CF₃, F, Cl, Br or I; and

R¹⁸ is alkyl, alkenyl, alkynyl, phenyl, heteroaryl, cycloalkyl, cycloalkenyl, heterocycloalkyl or heterocycloalkenyl.

Another embodiment of this invention, therefore pertains to a cationic lipid or mixtures thereof, having Formula (I)

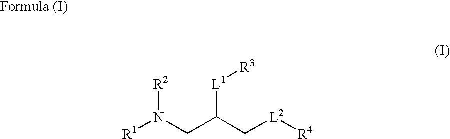

wherein

R¹ and R² are independently R⁵; or

R¹ and R², taken together with the atoms to which they are attached, are heterocycloalkyl or heteroaryl;

L¹ is O, or OC(O);

L² is O, or OC(O);

one of R³ and R⁴ is H, and the other is C₁₄-C₂₀-alkenyl; or

R³ and R⁴ are independently C₁₄-C₂₀-alkenyl or C₁₄-C₂₀-alkyl; or

R³ and R⁴ combine to form CR²⁰R²¹, wherein R²⁰ and R²¹ are the same or are different and are C₁₄-C₂₀-alkenyl;

R⁵ is alkyl, which is unsubstituted or substituted with R⁶;

R⁶ is R⁷;

R⁷ is phenyl;

wherein each foregoing cyclic moiety is independently unsubstituted or substituted with one or more R¹¹, N(R¹¹)₂, or OH;

R¹¹ is R¹², R¹³, or R¹⁵

R¹² is phenyl;

R¹³ is heteroaryl;

R¹⁵ is alkyl, which is unsubstituted or substituted with one or two of independently selected R¹⁶, or N(R¹⁶)₂;

R¹⁶ is alkyl, or R¹⁷; and

R¹⁷ is heterocycloalkyl.

One embodiment of this invention pertains to compounds of Formula (I), wherein R¹ and R² are each independently R⁵. In another embodiment of Formula (I), R¹ and R², taken together with the atoms to which they are attached, are heterocycloalkyl. In another embodiment of Formula (I), R¹ and R², taken together with the atoms to which they are attached, are heteroaryl. In another embodiment of Formula (I), R¹ and R², taken together with the atoms to which they are attached, are pyrrolidinyl, piperazinyl, 1,4-diazepanyl, piperidinyl, or morpholinyl. In another embodiment of Formula (I), R¹ and R², taken together with the atoms to which they are attached, are pyrrolidinyl. In another embodiment of Formula (I), R¹ and R², taken together with the atoms to which they are attached, are imidazolyl.

One embodiment of this invention pertains to compounds of Formula (I), wherein L¹ and L² are O. In another embodiment of Formula (I), L¹ and L² are OC(O). In another embodiment of Formula (I), L¹ and L² are (O)CO. In another embodiment of Formula (I), one of L¹ and L² is O, and the other is OC(O).

One embodiment of this invention pertains to compounds of Formula (I), wherein one of R³ and R⁴ is H, and the other is C₁₄-C₂₀-alkenyl. In another embodiment of Formula (I), one of R³ and R⁴ is H, and the other is C₁₄-C₂₀-alkyl. In another embodiment of Formula (I), R³ and R⁴ are C₁₄-C₂₀-alkenyl. In another embodiment of Formula (I), R³ and R⁴ are C₁₄-C₂₀-alkyl. In another embodiment of Formula (I), one of R³ and R⁴ is C₁₄-C₂₀-alkenyl, and the other is C₁₄-C₂₀-alkyl. In another embodiment of Formula (I), R³ and R⁴ combine to form CR²⁰R²¹, wherein R²⁰ and R²¹ are C₁₄-C₂₀-alkenyl. In another embodiment of Formula (I), R³ and R⁴ combine to form CR²⁰R²¹, wherein R²⁰ and R²¹ are C₁₄-C₂₀-alkyl.

One embodiment of this invention pertains to compounds of Formula (I), wherein R⁵ is alkyl, which is unsubstituted. In another embodiment of Formula (I), R⁵ is alkyl, which is substituted with R⁶. In another embodiment of Formula (I), R⁶ is phenyl which is unsubstituted.

One embodiment of Formula (I) pertains to compounds wherein all foregoing cyclic moieties are unsubstituted. In another embodiment of Formula (I), one or more cyclic moieties are substituted. In another embodiment of Formula (I), one or more cyclic moieties are substituted with one or more R¹¹, N(R¹¹)₂, or OH.

One embodiment of this invention pertains to compounds of Formula (I) wherein R¹ and R², taken together with the atoms to which they are attached, are heterocycloalkyl; L¹ and L² are O; and R³ and R⁴ are C₁₄-C₂₀-alkenyl. In another embodiment of Formula (I), R¹ and R², taken together with the atoms to which they are attached, are heteroaryl; L¹ and L² are O; and R³ and R⁴ are C₁₄-C₂₀-alkenyl. In another embodiment of Formula (I), R¹ and R², taken together with the atoms to which they are attached, are heterocycloalkyl; L¹ and L² are O; and R³ and R⁴ are C₁₄-C₂₀-alkenyl; wherein the heterocycloalkyl is substituted with R¹¹; R¹¹ is R¹⁵; and R¹⁵ is alkyl which is unsubstituted. In another embodiment of Formula (I), R¹ and R², taken together with the atoms to which they are attached, are heterocycloalkyl; L¹ and L² are O; and R³ and R⁴ are C₁₄-C₂₀-alkenyl; wherein the heterocycloalkyl is substituted with R¹¹; R¹¹ is R¹²; and R¹² is phenyl which is unfused. In another embodiment of Formula (I), R¹ and R², taken together with the atoms to which they are attached, are heterocycloalkyl; L¹ and L² are O; and R³ and R⁴ are C₁₄-C₂₀-alkenyl; wherein the heterocycloalkyl is substituted with R¹¹; R¹¹ is R¹³; and R¹³ is heteroaryl which is unfused. In another embodiment of Formula (I), R¹ and R² are each independently R⁵; wherein one R⁵ is alkyl which is unsubstituted, and the other R⁵ is alkyl which is substituted with R⁶; R⁶ is R⁷; R⁷ is phenyl which is unfused; L¹ and L² are O; and R³ and R⁴ are C₁₄-C₂₀-alkenyl. In another embodiment of Formula (I), R¹ and R², taken together with the atoms to which they are attached, are heterocycloalkyl; L¹ and L² are O; and R³ and R⁴ are C₁₄-C₂₀-alkenyl; wherein the heterocycloalkyl is substituted with R¹¹; R¹¹ is R¹⁵; R¹⁵ is alkyl which is substituted with N(R¹⁶)₂; and R¹⁶ is alkyl. In another embodiment of Formula (I), R¹ and R², taken together with the atoms to which they are attached, are heterocycloalkyl; L¹ and L² are O; and R³ and R⁴ are C₁₄-C₂₀-alkenyl; wherein the heterocycloalkyl is substituted with R¹¹; R¹¹ is R¹⁵; R¹⁵ is alkyl which is substituted with R¹⁶; R¹⁶ is R¹⁷; and R¹⁷ is heterocycloalkyl. In another embodiment of Formula (I), R¹ and R², taken together with the atoms to which they are attached, are heterocycloalkyl; L¹ and L² are O; and R³ and R⁴ are C₁₄-C₂₀-alkenyl; wherein the heterocycloalkyl is substituted with N(R¹¹)₂; R¹¹ is R¹⁵; and R¹⁵ is alkyl which is unsubstituted. In another embodiment of Formula (I), R¹ and R², taken together with the atoms to which they are attached, are heterocycloalkyl; L¹ and L² are O; and one of R³ and R⁴ is H, and the other is C₁₄-C₂₀-alkenyl. In another embodiment of Formula (I), R¹ and R², taken together with the atoms to which they are attached, are heterocycloalkyl; L¹ and L² are OC(O); and R³ and R⁴ are C₁₄-C₂₀-alkenyl. In another embodiment of Formula (I), R¹ and R², taken together with the atoms to which they are attached, are heterocycloalkyl; L¹ is O; L² is OC(O); and R³ and R⁴ are C₁₄-C₂₀-alkenyl. In another embodiment of Formula (I), R¹ and R², taken together with the atoms to which they are attached, are heterocycloalkyl; L¹ and L² are O; and R³ and R⁴ combine to form CR²⁰R²¹; wherein R²⁰ and R²¹ are C₁₄-C₂₀-alkenyl. In another embodiment of Formula (I), R¹ and R², taken together with the atoms to which they are attached, are heterocycloalkyl; L¹ and L² are O; and R³ and R⁴ are C₁₄-C₂₀-alkyl. In another embodiment of Formula (I), R¹ and R², taken together with the atoms to which they are attached, are heterocycloalkyl; L¹ and L² are O; and R³ and R⁴ are C₁₄-C₂₀-alkenyl; wherein the heterocycloalkyl is substituted OH. In another embodiment of Formula (I), R¹ and R², taken together with the atoms to which they are attached, are heterocycloalkyl; L¹ and L² are O; and R³ is C₁₄-C₂₀-alkyl and R⁴ is C₁₄-C₂₀-alkenyl.

Still another embodiment pertains to compounds of Formula I which are 1-(2,3-bis((9Z,12Z)-octadeca-9,12-dienyloxy)propyl)pyrrolidine, 1-(2,3-bis((9Z,12Z)-octadeca-9,12-dienyloxy)propyl)-1H-imidazole, 1-(2,3-bis((9Z,12Z)-octadeca-9,12-dienyloxy)propyl)-4-methylpiperazine, 1-(2,3-bis((9Z,12Z)-octadeca-9,12-dienyloxy)propyl)-4-methyl-1,4-diazepane, 1-(2,3-bis((9Z,12Z)-octadeca-9,12-dienyloxy)propyl)-4-phenylpiperazine, 1-(2,3-bis((9Z,12Z)-octadeca-9,12-dienyloxy)propyl)-4-pyridin-2-ylpiperazine, 1-(2,3-bis((9Z,12Z)-octadeca-9,12-dienyloxy)propyl)piperidine, 4-(2,3-bis((9Z,12Z)-octadeca-9,12-dienyloxy)propyl)morpholine, 1-((2R)-2,3-bis((9Z,12Z)-octadeca-9,12-dienyloxy)propyl)pyrrolidine, 1-((2S)-2,3-bis((9Z,12Z)-octadeca-9,12-dienyloxy)propyl)pyrrolidine, 1-(2,3-bis((9Z,12Z)-octadeca-9,12-dienyloxy)propyl)-4-ethylpiperazine, N-(2,3-bis((9Z,12Z)-octadeca-9,12-dienyloxy)propyl)-N-methyl-N-(3-(pyrrolidin-1-ylmethyl)benzyl)amine, N-(2-(4-(2,3-bis((9Z,12Z)-octadeca-9,12-dienyloxy)propyl)piperazin-1-yl)ethyl)-N,N-dimethylamine, 1-((2S)-2,3-bis((9Z,12Z)-octadeca-9,12-dienyloxy)propyl)-4-methylpiperazine, 1-((2R)-2,3-bis((9Z,12Z)-octadeca-9,12-dienyloxy)propyl)-4-methylpiperazine, 1-(2,3-bis((9Z,12Z)-octadeca-9,12-dienyloxy)propyl)-4-(2-pyrrolidin-1-ylethyl)piperazine, 2-(4-(2,3-bis((9Z,12Z)-octadeca-9,12-dienyloxy)propyl)piperazin-1-yl)pyrimidine, 1-(2,3-bis((9Z,12Z)-octadeca-9,12-dienyloxy)propyl)-N,N-diethylpyrrolidin-3-amine, 1-((9Z,12Z)-octadeca-9,12-dienyloxy)-3-pyrrolidin-1-ylpropan-2-ol, 2-[(9Z,12Z)-octadeca-9,12-dienoyloxy]-1-(pyrrolidin-1-ylmethyl)ethyl (9Z,12Z)-octadeca-9,12-dienoate, 2-[(9Z,12Z)-octadeca-9,12-dienyloxy]-1-(pyrrolidin-1-ylmethyl)ethyl (9Z,12Z)-octadeca-9,12-dienoate, 1-({2-[(8Z,11Z)-heptadeca-8,11-dienyl]-2-[(9Z,12Z)-octadeca-9,12-dienyl]-1,3-dioxolan-4-yl}methyl)pyrrolidine, 1-{2,3-bis[(5Z,8Z,11Z,14Z)-icosa-5,8,11,14-tetraenyloxy]propyl}pyrrolidine, 1-{3-[(5Z,8Z,11Z,14Z)-icosa-5,8,11,14-tetraenyloxy]-2-[(9Z,12Z)-octadeca-9,12-dienyloxy]propyl}pyrrolidine, 1-{2,3-bis[(9E,12E)-octadeca-9,12-dienyloxy]propyl}pyrrolidine, 1-{2-[(9E,12E)-octadeca-9,12-dienyloxy]-3-[(9Z,12Z)-octadeca-9,12-dienyloxy]propyl}pyrrolidine, 1-[2,3-bis(tetradecyloxy)propyl]pyrrolidine, 1-[2,3-bis(octadecyloxy)propyl]pyrrolidine, 1-{2,3-bis[(9Z)-octadec-9-enyloxy]propyl}pyrrolidine, 1-[2,3-bis(dodecyloxy)propyl]pyrrolidine, 1-{2,3-bis[(9Z,12Z)-octadeca-9,12-dienyloxy]propyl}pyrrolidin-3-ol, 1-{3-[(9Z,12Z)-hexadeca-9,12-dienyloxy]-2-[(9Z)-octadec-9-enyloxy]propyl}pyrrolidine, 1-{2,3-bis[(9Z,12Z)-octadeca-9,12-dienyloxy]propyl}-N,N-dimethylpyrrolidin-3-amine and 1-[3-[(9Z,12Z)-hexadeca-9,12-dienyloxy]-2-(tetradecyloxy)propyl]pyrrolidine.

Still another embodiment pertains to compounds of this invention wherein one or more cationic lipids are chosen from 1-(2,3-bis((9Z,12Z)-octadeca-9,12-dienyloxy)propyl)pyrrolidine, 1-(2,3-bis((9Z,12Z)-octadeca-9,12-dienyloxy)propyl)-4-methylpiperazine, N-(2-(4-(2,3-bis((9Z,12Z)-octadeca-9,12-dienyloxy)propyl)piperazin-1-yl)ethyl)-N,N-dimethylamine, 1-((2S)-2,3-bis((9Z,12Z)-octadeca-9,12-dienyloxy)propyl)-4-methylpiperazine, 1-((2R)-2,3-bis((9Z,12Z)-octadeca-9,12-dienyloxy)propyl)-4-methylpiperazine, 1-(2,3-bis((9Z,12Z)-octadeca-9,12-dienyloxy)propyl)-4-(2-pyrrolidin-1-ylethyl)piperazine, 1-{2,3-bis[(9Z,12Z)-octadeca-9,12-dienyloxy]propyl}-N,N-dimethylpyrrolidin-3-amine, 1-{2,3-bis[(9E,12E)-octadeca-9,12-dienyloxy]propyl}pyrrolidine, 1-{2-[(9E,12E)-octadeca-9,12-dienyloxy]-3-[(9Z,12Z)-octadeca-9,12-dienyloxy]propyl}pyrrolidine, 1-{2,3-bis[(9Z)-octadec-9-enyloxy]propyl}pyrrolidine, 1-{2,3-bis[(9Z,12Z)-octadeca-9,12-dienyloxy]propyl}pyrrolidin-3-ol, and 1-{3-[(9Z,12Z)-hexadeca-9,12-dienyloxy]-2-[(9Z)-octadec-9-enyloxy]propyl}pyrrolidine.

Still another embodiment pertains to compounds of this invention wherein one or more cationic lipids are chosen from 1-(2,3-bis((9Z,12Z)-octadeca-9,12-dienyloxy)propyl)pyrrolidine, 1-(2,3-bis((9Z,12Z)-octadeca-9,12-dienyloxy)propyl)-4-methylpiperazine, 1-((2R)-2,3-bis((9Z,12Z)-octadeca-9,12-dienyloxy)propyl)pyrrolidine, 1-((2S)-2,3-bis((9Z,12Z)-octadeca-9,12-dienyloxy)propyl)pyrrolidine, and 1-{2-[(9E,12E)-octadeca-9,12-dienyloxy]-3-[(9Z,12Z)-octadeca-9,12-dienyloxy]propyl}pyrrolidine.

### Particles, Cationic-Based Lipid Encapsulation Systems, and Lipid-Based Particles

A further embodiment pertains to particles comprising one or more cationic lipid(s) having Formula I.

A further embodiment pertains to particles comprising one or more cationic lipid(s) having Formula I and one or more therapeutic agents. Preferably said therapeutic agent is a nucleic acid encoded with a product of interest, including but not limited to, RNA, antisense oligonucleotide, a DNA, a plasmid, a ribosomal RNA (rRNA), a micro RNA (miRNA), transfer RNA (tRNA), a small inhibitory RNA (siRNA), small nuclear RNA (snRNA), antigens, fragments thereof, proteins, peptides, and small-molecules.

A further embodiment pertains to nanoparticles comprising one or more cationic lipid(s) having Formula I.

A further embodiment pertains to nanoparticles comprising one or more cationic lipid(s) having Formula I and one or more therapeutic agents. Preferably said therapeutic agent is a nucleic acid encoded with a product of interest, including but not limited to, RNA, antisense oligonucleotide, a DNA, a plasmid, a ribosomal RNA (rRNA), a micro RNA (miRNA), transfer RNA (tRNA), a small inhibitory RNA (siRNA), small nuclear RNA (snRNA), antigens, fragments thereof, proteins, peptides, and small-molecules.

A still further embodiment pertains to Cationic-Based Lipid Encapsulation Systems (CaBLES) comprising non-cationic lipid(s), polyethylene glycol (PEG)-lipid conjugate(s) and cationic lipid(s) having Formula I.

A still further embodiment pertains to Cationic-Based Lipid Encapsulation Systems (CaBLES) comprising one or more cationic lipids having Formula (I)

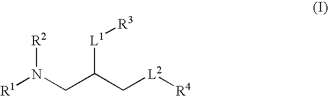

wherein

R¹ and R² are independently H, cycloalkyl, cycloalkenyl or R⁵; or

R¹ and R², taken together with the atoms to which they are attached, are heterocycloalkyl or heteroaryl;

L¹ is O, OC(O) or (O)CO;

L² is O, OC(O) or (O)CO;

one of R³ and R⁴ is H, and the other is C₁₄-C₂₀-alkenyl or C₁₄-C₂₀-alkyl; or

R³ and R⁴ are independently C₁₄-C₂₀-alkenyl or C₁₄-C₂₀-alkyl; or

R³ and R⁴ combine to form CR²⁰R²¹, wherein R²⁰ is H and R²¹ is C₁₄-C₂₀-alkenyl or C₁₄-C₂₀-alkyl; or R²⁰ and R²¹ are the same or are different and are C₁₄-C₂₀-alkenyl, C₁₄-C₂₀-alkyl, or (CH₂O)—C₁₄-C₂₀ alkenyl;

R⁵ is alkyl, which is unsubstituted or substituted with one or more R⁶, OR⁶, SR⁶, S(O)R⁶, SO₂R⁶, C(O)R⁶, CO(O)R⁶, OC(O)R⁶, OC(O)OR⁶, NH₂, NHR⁶, N(R⁶)₂, NHC(O)R⁶, NR⁶C(O)R⁶, NHS(O)₂R⁶, N R⁶S(O)₂R⁶, NHC(O)OR⁶, NR⁶C(O)OR⁶, NHC(O)NH₂, NHC(O)NHR⁶, NHC(O)N(R⁶)₂, NR⁶C(O)NHR⁶, NR⁶C(O)N(R⁶)₂, C(O)NH₂, C(O)NHR⁶, C(O)N(R⁶)₂, C(O)NHOH, C(O)NHOR⁶, C(O)NHSO₂R⁶, C(O)NR⁶SO₂R⁶, SO₂NH₂, SO₂NHR⁶, SO₂N(R⁶)₂, C(O)H, C(O)OH, C(N)NH₂, C(N)NHR⁶, C(N)N(R⁶)₂, CNOH, CNOCH₃, OH, (O), CN, N₃, NO₂, CF₃, CF₂CF₃, OCF₃, OCF₂CF₃, F, Cl, Br or I;

R⁶ is R⁷, R⁸, R⁹, or R¹⁰;

R⁷ is phenyl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R⁸ is heteroaryl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R⁹ is cycloalkyl, cycloalkenyl, heterocycloalkyl or heterocycloalkenyl, each of which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹⁰ is alkyl, alkenyl or alkynyl;

wherein each foregoing cyclic moiety is independently unsubstituted or substituted with one or more R¹¹,OR¹¹,SR²¹, S(O)R²¹, SO₂R¹¹, C(O)R¹¹, CO(O)R¹¹, OC(O)R¹¹OC(O)OR¹¹, NH, NHR¹¹, N(R¹¹)₂, NHC(O)R¹¹, NR¹¹C(O)R¹¹, NHS(O)₂R¹¹, N, R¹¹S(O)₂R¹¹, NHC(O)OR¹¹, NR¹¹C(O)OR¹¹, NHC(O)NH₂, NHC(O)NHR¹¹, NHC(O)N(R¹¹), NR¹¹C(O)NHR¹¹, NR¹¹C(O)N(R¹¹), C(O)NH, C(O)NHR¹¹, C(O)N(R¹¹)₂, C(O)NHOH, C(O)NHOR¹¹, C(O)NHSO₂R¹¹, C(O)NR¹¹SO₂R¹¹, SO₂NH₂, SO₂NHR¹¹, SO₂N(R¹¹)₂, C(O)H, C(O)OH, C(N)NH₂, C(N)NHR¹¹, C(N)N(R¹¹)₂, CNOH, CNOCH₃, OH, (O), CN, N₃, NO₂, CF₃, CF₂CF₃, OCF₃, OCF₂CF₃, F, Cl, Br or I;

R¹¹ is R¹², R¹³, R¹⁴, or R¹⁵;

R¹² is phenyl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹³ is heteroaryl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹⁴ is cycloalkyl, cycloalkenyl, heterocycloalkyl or heterocycloalkenyl, each of which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹⁵ is alkyl, alkenyl or alkynyl, each of which is unsubstituted or substituted with one or two of independently selected R¹⁶, OR¹⁶, SR¹⁶, S(O)₂R¹⁶, C(O)OH, NH₂, NHR¹⁶N(R⁶¹)₂, C(O)R¹⁶, C(O)NH, C(O)NHR¹⁶, C(O)N(R¹⁶), NHC(O)R¹⁶, NR¹⁶C(O)R¹⁶, NHC(O)OR¹⁶, NR¹⁶C(O)OR¹⁶, OH, F, Cl, Br or I;

R¹⁶ is alkyl, alkenyl, alkynyl, or R¹⁷

R¹⁷ is phenyl, heteroaryl, cycloalkyl, cycloalkenyl, heterocycloalkyl or heterocycloalkenyl, each of which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

wherein R¹², R¹³, R¹⁴, and R¹⁷ are independently unsubstituted or substituted with one or more R¹⁸,OR¹⁸, SR¹⁸,S(O)R¹⁸, SO₂R¹⁸, C(O)R¹⁸, CO(O)R¹⁸, OC(O)R¹⁸, OC(O)OR¹⁸, NH₂, NHR¹⁸, N(R¹⁸)₂, NHC(O)R¹⁸, NR¹⁸C(O)R¹⁸, NHS(O)₂R¹⁸, NR¹⁸S(O)₂R¹⁸, NHC(O)OR¹⁸, NR¹⁸C(O)OR¹⁸, NHC(O)NH₂, NHC(O)NHR¹⁸, NHC(O)N(R¹⁸)₂, NR¹⁸C(O)NHR¹⁸, NR¹⁸C(O)N(R¹⁸)₂, C(O)NH₂, C(O)NHR¹⁸, C(O)N(R¹⁸)₂, C(O)NHOH, C(O)NHOR¹⁸, C(O)NHSO₂R¹⁸, C(O)NR¹⁸SO₂R¹⁸, SO₂NH₂, SO₂NHR¹⁸, SO₂N(R¹⁸)₂, C(O)H, C(O)OH, C(N)NH₂, C(N)NHR¹⁸, C(N)N(R¹⁸)₂, CNOH, CNOCH₃, OH, (O), CN, N₃, NO₂, CF₃, CF₂CF₃, OCF₃, OCF₂CF₃, F, Cl, Br or I; and

R¹⁸ is alkyl, alkenyl, alkynyl, phenyl, heteroaryl, cycloalkyl, cycloalkenyl, heterocycloalkyl or heterocycloalkenyl; and

one or more non-cationic lipids, and one or more polyethylene glycol-lipid conjugates.

In still a further embodiment, Lipid-Based Particles of the present invention are defined as CaBLES which further comprise one or more therapeutic agent(s). Therapeutic agents that can be delivered with CaBLES include RNA, antisense oligonucleotide, a DNA, a plasmid, a ribosomal RNA (rRNA), a micro RNA (miRNA), transfer RNA (tRNA), a small inhibitory RNA (siRNA), small nuclear RNA (snRNA), chimeric nucleic acids, an antigen, fragments thereof, a protein, a peptide, small-molecules, or mixtures thereof. This invention describes delivery of RNA's such as small inhibitory RNA or microRNA. The nucleic acid can have varying lengths (10-200 bps) and structures (hairpins, single/double strands, bulges, nicks/gaps, mismatches) and processed in the cell to provide active gene silencing. In certain embodiments of this invention, a double-stranded siRNA (dsRNA) can have the same number of nucleotides on each strand (blunt ends) or asymmetric ends (overhangs). The overhang of 1-2 nucleotides can be present on the sense and/or the antisense strand, as well as present on the 5′- and/or the 3′-ends of a given strand.

In certain embodiments, the CaBLES and/or the Lipid-Based Particle formulation can have a ligand attached, such as a targeting ligand or a chelating moiety for complexing calcium. Preferably, after the ligand is attached, the cationic lipids of Formula I maintains a positive charge. In certain instances, the ligand that is attached has a positive charge. Suitable ligands include, but are not limited to, a compound or device with a reactive functional group and include lipids, amphipathic lipids, carrier compounds, bioaffinity compounds, biomaterials, biopolymers, biomedical devices, analytically detectable compounds, therapeutically active compounds, enzymes, peptides, proteins, antibodies, immune stimulators, radiolabels, fluorogens, biotin, drugs, haptens, DNA, RNA, polysaccharides, liposomes, virosomes, micelles, immunoglobulins, functional groups, other targeting moieties, or toxins.

In another embodiment, a targeting ligand (moiety) is conjugated to the periphery of the PEG-lipid in a Lipid-Based Particle formulation. Preferably, the targeting moiety is a ligand of a receptor present on a target cell and the receptor is preferentially expressed by the target cell versus a non-target cell. In one aspect, the targeting moiety is an antibody or fragments thereof. In one aspect, the targeting moiety is a small protein, or peptide. In another aspect, the targeting moiety is a small-molecule.

In still a further embodiment, these Lipid-Based Particles are nanoparticles and have mean diameter sizes of about 50-300 nm, of which 50-250 nm is preferred and 50-200 nm is most preferred.

A further embodiment pertains to CaBLES or Lipid-Base Particles wherein the PEG lipid conjugate is about 0.1-20 weight/weight % of total lipid in particle, the non-cationic lipid is about 1-30 weight/weight % of total lipid in particle, the cholesterol is about 5-45 weight/weight % of total lipid in particle, and the cationic lipid is about 5-60 weight/weight % of total lipid in particle.

A further embodiment pertains to CaBLES or Lipid-Base Particles wherein the PEG lipid conjugate is about 0.1-20 weight/weight % of total lipid in particle, the DSPC is about 1-30 weight/weight % of total lipid in particle, the cholesterol is about 5-45 weight/weight % of total lipid in particle, and the cationic lipid is about 5-60 weight/weight % of total lipid in particle.

A further embodiment pertains to a pharmaceutical composition comprising a Lipid-Based Particle and a pharmaceutically acceptable carrier.

A further embodiment pertains to a pharmaceutical composition, wherein the Lipid-Based Particle comprises cholesterol, DSPC, 1-(2,3-bis((9Z,12Z)-octadeca-9,12-dienyloxy)propyl)pyrrolidine, one or more PEG-lipid conjugates, and one or more nucleic acids.

A further embodiment pertains to a pharmaceutical composition, wherein the (PEG)-lipid conjugates are about 0.1-20 weight/weight % of total lipid in particle, the DSPC is about 1-30 weight/weight % of total lipid in particle, the cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugate is N-(2,3-dimyristyloxypropyl)carbamate polyethyleneglycol-2000 methyl ether, and the therapeutic agent is siRNA.

A further embodiment pertains to a Lipid-Based Particle, wherein the N-(2,3-dimyristyloxypropyl)carbamate polyethyleneglycol-2000 methyl ether is about 0.1-20 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugate is N-(carbonyl-methoxypolyethyleneglycol-2000)-1,2-distearoyl-sn-glycero-3-phosphoethanolamine, and the therapeutic agent is siRNA.

A further embodiment pertains to a Lipid-Based Particle, wherein the N-(carbonyl-methoxypolyethyleneglycol-2000)-1,2-distearoyl-sn-glycero-3-phosphoethanolamine is about 0.1-20 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugate is N-(carbonyl-methoxypolyethyleneglycol-2000)-1,2-dipalmitoyl-sn-glycero-3-phosphoethanolamine, and the therapeutic agent is siRNA.

A further embodiment pertains to a Lipid-Based Particle, wherein the N-(carbonyl-methoxypolyethyleneglycol-2000)-1,2-dipalmitoyl-sn-glycero-3-phosphoethanolamine is about 0.1-20 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugate is N-(carbonyl-methoxypolyethyleneglycol-2000)-1,2-dimyristoyl-sn-glycero-3-phosphoethanolamine, and the therapeutic agent is siRNA.

A further embodiment pertains to a Lipid-Based Particle, wherein the N-(carbonyl-methoxypolyethyleneglycol-2000)-1,2-dimyristoyl-sn-glycero-3-phosphoethanolamine is about 0.1-20 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugate is 1,2-distearoyl-sn-glycerol-methoxypolyethyleneglycol-2000, and the therapeutic agent is siRNA.

A further embodiment pertains to a Lipid-Based Particle, wherein the 1,2-distearoyl-sn-glycerol-methoxypolyethyleneglycol-2000 is about 0.1-20 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3bis((9Z,12Z)-octadeca-9,12-dienyloxy)propyl)pyrrolidine, the PEG-lipid conjugate is 1,2-dipalmitoyl-sn-glycerol-methoxypolyethyleneglycol-2000, and the therapeutic agent is siRNA.

A further embodiment pertains to a Lipid-Based Particle, wherein the 1,2-dipalmitoyl-sn-glycerol-methoxypolyethyleneglycol-2000 is about 0.1-20 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugate is 1,2-dimyristoyl-sn-glycerol-methoxypolyethyleneglycol-2000, and the therapeutic agent is siRNA.

A further embodiment pertains to a Lipid-Based Particle, wherein the 1,2-dimyristoyl-sn-glycerol-methoxypolyethyleneglycol-2000 is about 0.1-20 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugate is N-[3,4-bis(tetradecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide, and the therapeutic agent is siRNA.

A further embodiment pertains to a Lipid-Based Particle, wherein the N-[3,4-bis(tetradecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide is about 0.1-20 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugate is N-[3,4-bis(hexadecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide, and the therapeutic agent is siRNA.

A further embodiment pertains to a Lipid-Based Particle, wherein the N-[3,4-bis(hexadecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide is about 0.1-20 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a pharmaceutical composition, wherein the Lipid-Based Particle comprises, cholesterol, SPC, 1-(2,3-bis((9Z,12Z)-octadeca-9,12-dienyloxy)propyl)pyrrolidine, one or more PEG-lipid conjugates, and one or more nucleic acids.

A further embodiment pertains to a pharmaceutical composition, wherein the (PEG)-lipid conjugates are about 0.1-20 weight/weight % of total lipid in particle, the SPC is about 1-30 weight/weight % of total lipid in particle, the cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and SPC, the cationic lipid is 1-(2,3-bis((9Z,12Z)-octadeca-9,12-dienyloxy)propyl)pyrrolidine, the PEG-lipid conjugate is 1,2-dipalmitoyl-sn-glycerol-methoxypolyethyleneglycol-2000 and, and the therapeutic agent is siRNA.

A further embodiment pertains to a Lipid-Based Particle, wherein the 1,2-dipalmitoyl-sn-glycerol-methoxypolyethyleneglycol-2000 is about 0.1-20 weight/weight % of total lipid in particle, the SPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugates are 1,2-dimyristoyl-sn-glycerol-methoxypolyethyleneglycol-2000 and 1,2-dipalmitoyl-sn-glycerol-methoxypolyethyleneglycol-2000, and the therapeutic agent is siRNA.

A further embodiment pertains to a Lipid-Based Particle, wherein the 1,2-dimyristoyl-sn-glycerol-methoxypolyethyleneglycol-2000 and 1,2-dipalmitoyl-sn-glycerol-methoxypolyethyleneglycol-2000 are about 0.1-20 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugates are 1,2-dimyristoyl-sn-glycerol-methoxypolyethyleneglycol-2000 and 1,2-distearoyl-sn-glycerol-methoxypolyethyleneglycol-2000, and the therapeutic agent is siRNA.

A further embodiment pertains to a Lipid-Based Particle, wherein the 1,2-dimyristoyl-sn-glycerol-methoxypolyethyleneglycol-2000 and 1,2-distearoyl-sn-glycerol-methoxypolyethyleneglycol-2000 are about 0.1-20 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugates are 1,2-dipalmitoyl-sn-glycerol-methoxypolyethyleneglycol-2000 and 1,2-distearoyl-sn-glycerol-methoxypolyethyleneglycol-2000, and the therapeutic agent is siRNA.

A further embodiment pertains to a Lipid-Based Particle, wherein the 1,2-dipalmitoyl-sn-glycerol-methoxypolyethyleneglycol-2000 and 1,2-distearoyl-sn-glycerol-methoxypolyethyleneglycol-2000 are about 0.1-20 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugates are N-(carbonyl-methoxypolyethyleneglycol-2000)-1,2-dimyristoyl-sn-glycero-3-phosphoethanolamine and N-(carbonyl-methoxypolyethyleneglycol-2000)-1,2-dipalmitoyl-sn-glycero-3-phosphoethanolamine, and the therapeutic agent is siRNA.

A further embodiment pertains to a Lipid-Based Particle, wherein the N-(carbonyl-methoxypolyethyleneglycol-2000)-1,2-dimyristoyl-sn-glycero-3-phosphoethanolamine and N-(carbonyl-methoxypolyethyleneglycol-2000)-1,2-dipalmitoyl-sn-glycero-3-phosphoethanolamine are about 0.1-20 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugates are N-(carbonyl-methoxypolyethyleneglycol-2000)-1,2-distearoyl-sn-glycero-3-phosphoethanolamine and N-(carbonyl-methoxypolyethyleneglycol-2000)-1,2-dipalmitoyl-sn-glycero-3-phosphoethanolamine, and the therapeutic agent is siRNA.

A further embodiment pertains to a Lipid-Based Particle, wherein the N-(carbonyl-methoxypolyethyleneglycol-2000)-1,2-distearoyl-sn-glycero-3-phosphoethanolamine and N-(carbonyl-methoxypolyethyleneglycol-2000)-1,2-dipalmitoyl-sn-glycero-3-phosphoethanolamine are about 0.1-20 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugates are N-[3,4-bis(hexadecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide and 1,2-distearoyl-sn-glycerol-methoxypolyethyleneglycol-2000, and the therapeutic agent is siRNA.

A further embodiment pertains to a Lipid-Based Particle, wherein the N-[3,4-bis(hexadecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide and 1,2-distearoyl-sn-glycerol-methoxypolyethyleneglycol-2000 are about 0.1-20 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugates are N-(carbonyl-methoxypolyethyleneglycol-2000)-1,2-dimyristoyl-sn-glycero-3-phosphoethanolamine and N-(carbonyl-methoxypolyethyleneglycol-2000)-1,2-distearoyl-sn-glycero-3-phosphoethanolamine, and the therapeutic agent is siRNA.

A further embodiment pertains to a Lipid-Based Particle, wherein the N-(carbonyl-methoxypolyethyleneglycol-2000)-1,2-dimyristoyl-sn-glycero-3-phosphoethanolamine and N-(carbonyl-methoxypolyethyleneglycol-2000)-1,2-distearoyl-sn-glycero-3-phosphoethanolamine are about 0.1-20 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugates are 1,2-dimyristoyl-sn-glycerol-methoxypolyethyleneglycol-2000 and N-(carbonyl-methoxypolyethyleneglycol-2000)-1,2-distearoyl-sn-glycero-3-phosphoethanolamine, and the therapeutic agent is siRNA.

A further embodiment pertains to a Lipid-Based Particle, wherein the 1,2-dimyristoyl-sn-glycerol-methoxypolyethyleneglycol-2000 and N-(carbonyl-methoxypolyethyleneglycol-2000)-1,2-distearoyl-sn-glycero-3-phosphoethanolamine are about 0.1-20 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugates are 1,2-distearoyl-sn-glycerol-methoxypolyethyleneglycol-2000 and N-(carbonyl-methoxypolyethyleneglycol-2000)-1,2-dimyristoyl-sn-glycero-3-phosphoethanolamine, and the therapeutic agent is siRNA.

A further embodiment pertains to a Lipid-Based Particle, wherein the 1,2-distearoyl-sn-glycerol-methoxypolyethyleneglycol-2000 and N-(carbonyl-methoxypolyethyleneglycol-2000)-1,2-dimyristoyl-sn-glycero-3-phosphoethanolamine are about 0.1-20 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugates are N-[3,4-bis(hexadecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide and N-(carbonyl-methoxypolyethyleneglycol-2000)-1,2-distearoyl-sn-glycero-3-phosphoethanolamine, and the therapeutic agent is siRNA.

A further embodiment pertains to a Lipid-Based Particle, wherein the N-[3,4-bis(hexadecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide and N-(carbonyl-methoxypolyethyleneglycol-2000)-1,2-distearoyl-sn-glycero-3-phosphoethanolamine are about 0.1-20 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugates are N-(carbonyl-methoxypolyethyleneglycol-2000)-1,2-dimyristoyl-sn-glycero-3-phosphoethanolamine and 1,2-dipalmitoyl-sn-glycerol-methoxypolyethyleneglycol-2000, and the therapeutic agent is siRNA.

A further embodiment pertains to a Lipid-Based Particle, wherein the N-(carbonyl-methoxypolyethyleneglycol-2000)-1,2-dimyristoyl-sn-glycero-3-phosphoethanolamine and 1,2-dipalmitoyl-sn-glycerol-methoxypolyethyleneglycol-2000 are about 0.1-20 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a pharmaceutical composition, wherein the Lipid-Based Particle comprises, cholesterol, DSPC, 1-(2,3-bis((9Z,12Z)-octadeca-9,12-dienyloxy)propyl)-4-methylpiperazine, one or more PEG-lipid conjugates, and one or more nucleic acids.

A further embodiment pertains to a pharmaceutical composition, wherein the (PEG)-lipid conjugates are about 0.1-20 weight/weight % of total lipid in particle, the DSPC is about 1-30 weight/weight % of total lipid in particle, the cholesterol is about 5-45 weight/weight % of total lipid in particle, and 1-(2,3-bis((9Z,12Z)-octadeca-9,12-dienyloxy)propyl)-4-methylpiperazine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)-4-methylpiperazine, the PEG-lipid conjugate is N-(2,3-dimyristyloxypropyl)carbamate polyethyleneglycol-2000 methyl ether, and the therapeutic agent is siRNA.

A further embodiment pertains to a Lipid-Based Particle, wherein the N-(2,3-dimyristyloxypropyl)carbamate polyethyleneglycol-2000 methyl ether is about 0.1-20 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)-4-methylpiperazine is about 5-60 weight/weight % of total lipid in particle.

A further embodiment pertains to a pharmaceutical composition, wherein the Lipid-Based Particle comprises, cholesterol, DSPC, 1-((2S)-2,3-bis((9Z,12Z)-octadeca-9,12-dienyloxy)propyl)pyrrolidine, one or more PEG-lipid conjugates, and one or more nucleic acids.

A further embodiment pertains to a pharmaceutical composition, wherein the (PEG)-lipid conjugates are about 0.1-20 weight/weight % of total lipid in particle, the DSPC is about 1-30 weight/weight % of total lipid in particle, the cholesterol is about 5-45 weight/weight % of total lipid in particle, and 1-((2S)-2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-((2S)-2,3-bis((9Z,12Z)-octadeca-9,12-dienyloxy)propyl)pyrrolidine, the PEG-lipid conjugate is N-(2,3-dimyristyloxypropyl)carbamate polyethyleneglycol-2000 methyl ether, and the therapeutic agent is siRNA.

A further embodiment pertains to a Lipid-Based Particle, wherein the N-(2,3-dimyristyloxypropyl)carbamate polyethyleneglycol-2000 methyl ether is about 0.1-20 weight/weight % of total lipid in particle, the DSPC is about 1-30 weight/weight % of total lipid in particle, the cholesterol is about 5-45 weight/weight % of total lipid in particle, and the 1-((2S)-2,3-bis((9Z,12Z)-octadeca-9,12-dienyloxy)propyl)pyrrolidine is about 5-60 weight/weight % of total lipid in particle.

A further embodiment pertains to a pharmaceutical composition, wherein the Lipid-Based Particle comprises, cholesterol, DSPC, 1-((2R)-2,3-bis((9Z,12Z)-octadeca-9,12-dienyloxy)propyl)pyrrolidine, one or more PEG-lipid conjugates, and one or more nucleic acids.

A further embodiment pertains to a pharmaceutical composition, wherein the (PEG)-lipid conjugates are about 0.1-20 weight/weight % of total lipid in particle, the DSPC is about 1-30 weight/weight % of total lipid in particle, the cholesterol is about 5-45 weight/weight % of total lipid in particle, and 1-((2R)-2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-((2R)-2,3-bis((9Z,12Z)-octadeca-9,12-dienyloxy)propyl)pyrrolidine, the PEG-lipid conjugate is N-(2,3-dimyristyloxypropyl)carbamate polyethyleneglycol-2000 methyl ether, and the therapeutic agent is siRNA.

A further embodiment pertains to a Lipid-Based Particle, wherein the N-(2,3-dimyristyloxypropyl)carbamate polyethyleneglycol-2000 methyl ether is about 0.1-20 weight/weight % of total lipid in particle, the DSPC is about 1-30 weight/weight % of total lipid in particle, the cholesterol is about 5-45 weight/weight % of total lipid in particle, and the 1-((2R)-2,3-bis((9Z,12Z)-octadeca-9,12-dienyloxy)propyl)pyrrolidine is about 5-60 weight/weight % of total lipid in particle.

A further embodiment pertains to a pharmaceutical composition, wherein the Lipid-Based Particle comprises, cholesterol, DSPC, 1-{2-[(9E,12E)-octadeca-9,12-dienyloxy]-3-[(9Z,12Z)-octadeca-9,12-dienyloxy]propyl}pyrrolidine, one or more PEG-lipid conjugates, and one or more nucleic acids.

A further embodiment pertains to a pharmaceutical composition, wherein the (PEG)-lipid conjugates are about 0.1-20 weight/weight % of total lipid in particle, the DSPC is about 1-30 weight/weight % of total lipid in particle, the cholesterol is about 5-45 weight/weight % of total lipid in particle, and 1-{2-[(9E,12E)-octadeca-9,12-dienyloxy]-3-[(9Z,12Z)-octadeca-9,12-dienyloxy]propyl}pyrrolidine is about 5-60 weight/weight % of total lipid in particle.

A further embodiment pertains to a Lipid-Based Particle, wherein the non-cationic lipids are cholesterol and DSPC, the cationic lipid is 1-{2-[(9E,12E)-octadeca-9,12-dienyloxy]-3-[(9Z,12Z)-octadeca-9,12-dienyloxy]propyl}pyrrolidine, the PEG-lipid conjugate is 1,2-dipalmitoyl-sn-glycerol-methoxypolyethyleneglycol-2000, and the therapeutic agent is siRNA.

A further embodiment pertains to a Lipid-Based Particle, wherein the 1,2-dipalmitoyl-sn-glycerol-methoxypolyethyleneglycol-2000 is about 0.1-20 weight/weight % of total lipid in particle, the DSPC is about 1-30 weight/weight % of total lipid in particle, the cholesterol is about 5-45 weight/weight % of total lipid in particle, and the 1-{2-[(9E,12E)-octadeca-9,12-dienyloxy]-3-[(9Z,12Z)-octadeca-9,12-dienyloxy]propyl}pyrrolidine is about 5-60 weight/weight % of total lipid in particle.

A further embodiment pertains to Lipid-Based Particles, wherein the ratio of one or more (PEG)-lipid conjugates, one or more non-cationic lipids, and one or more cationic lipids of Formula (I), to one or more therapeutic agents is between about 50:1 to about 5:1.

The A further embodiment pertains to Lipid-Based Particles, wherein the ratio of one or more (PEG)-lipid conjugates, one or more non-cationic lipids, and one or more cationic lipids of Formula (I), to one or more therapeutic agents is between about 30:1 to about 10:1.

In still a further embodiment, functional CaBLES comprising one or more (PEG)-lipid conjugates, one or more non-cationic lipids, and one or more cationic lipids of Formula I effectively encapsulate nucleic acids, such as siRNA, with efficiencies from about 50-100%.

In still a further embodiment, functional CaBLES comprising one or more (PEG)-lipid conjugates, one or more non-cationic lipids, and one or more cationic lipids of Formula 1 effectively encapsulate nucleic acids, such as siRNA, with efficiencies from about 80-100%.

In still a further embodiment, functional CaBLES comprising one or more (PEG)-lipid conjugates, one or more non-cationic lipids, and one or more cationic lipids chosen from 1-(2,3-bis((9Z,12Z)-octadeca-9,12-dienyloxy)propyl)pyrrolidine, 1-(2,3-bis((9Z,12Z)-octadeca-9,12-dienyloxy)propyl)-1H-imidazole, 1-(2,3-bis((9Z,12Z)-octadeca-9,12-dienyloxy)propyl)-4-methylpiperazine, 1-(2,3-bis((9Z,12Z)-octadeca-9,12-dienyloxy)propyl)-4-methyl-1,4-diazepane, 1-(2,3-bis((9Z,12Z)-octadeca-9,12-dienyloxy)propyl)-4-phenylpiperazine, 1-(2,3-bis((9Z,12Z)-octadeca-9,12-dienyloxy)propyl)-4-pyridin-2-ylpiperazine, 1-(2,3-bis((9Z,12Z)-octadeca-9,12-dienyloxy)propyl)piperidine, 4-(2,3-bis((9Z,12Z)-octadeca-9,12-dienyloxy)propyl)morpholine, 1-((2R)-2,3-bis((9Z,12Z)-octadeca-9,12-dienyloxy)propyl)pyrrolidine, 1-((2S)-2,3-bis((9Z,12Z)-octadeca-9,12-dienyloxy)propyl)pyrrolidine, 1-(2,3-bis((9Z,12Z)-octadeca-9,12-dienyloxy)propyl)-4-ethylpiperazine, N-(2,3-bis((9Z,12Z)-octadeca-9,12-dienyloxy)propyl)-N-methyl-N-(3-(pyrrolidin-1-ylmethyl)benzyl)amine, N-(2-(4-(2,3-bis((9Z,12Z)-octadeca-9,12-dienyloxy)propyl)piperazin-1-yl)ethyl)-N,N-dimethylamine, 1-((2S)-2,3-bis((9Z,12Z)-octadeca-9,12-dienyloxy)propyl)-4-methylpiperazine, 1-((2R)-2,3-bis((9Z,12Z)-octadeca-9,12-dienyloxy)propyl)-4-methylpiperazine, 1-(2,3-bis((9Z,12Z)-octadeca-9,12-dienyloxy)propyl)-4-(2-pyrrolidin-1-ylethyl)piperazine, 2-(4-(2,3-bis((9Z,12Z)-octadeca-9,12-dienyloxy)propyl)piperazin-1-yl)pyrimidine, 1-(2,3-bis((9Z,12Z)-octadeca-9,12-dienyloxy)propyl)-N,N-diethylpyrrolidin-3-amine, 1-((9Z,12Z)-octadeca-9,12-dienyloxy)-3-pyrrolidin-1-ylpropan-2-ol, 2-[(9Z,12Z)-octadeca-9,12-dienoyloxy]-1-(pyrrolidin-1-ylmethyl)ethyl (9Z,12Z)-octadeca-9,12-dienoate, 2-[(9Z,12Z)-octadeca-9,12-dienyloxy]-1-(pyrrolidin-1-ylmethyl)ethyl (9Z,12Z)-octadeca-9,12-dienoate, 1-({2-[(8Z,11Z)-heptadeca-8,11-dienyl]-2-[(9Z,12Z)-octadeca-9,12-dienyl]-1,3-dioxolan-4-yl}methyl)pyrrolidine, 1-{2,3-bis[(5Z,8Z,11Z,14Z)-icosa-5,8,11,14-tetraenyloxy]propyl}pyrrolidine, 1-{3-[(5Z,8Z,11Z,14Z)-icosa-5,8,11,14-tetraenyloxy]-2-[(9Z,12Z)-octadeca-9,12-dienyloxy]propyl}pyrrolidine, 1-{2,3-bis[(9E,12E)-octadeca-9,12-dienyloxy]propyl}pyrrolidine, 1-{2-[(9E,12E)-octadeca-9,12-dienyloxy]-3-[(9Z,12Z)-octadeca-9,12-dienyloxy]propyl}pyrrolidine, 1-[2,3-bis(tetradecyloxy)propyl]pyrrolidine, 1-[2,3-bis(octadecyloxy)propyl]pyrrolidine, 1-{2,3-bis[(9Z)-octadec-9-enyloxy]propyl}pyrrolidine, 1-[2,3-bis(dodecyloxy)propyl]pyrrolidine, 1-{2,3-bis[(9Z,12Z)-octadeca-9,12-dienyloxy]propyl}pyrrolidin-3-ol, 1-{3-[(9Z,12Z)-hexadeca-9,12-dienyloxy]-2-[(9Z)-octadec-9-enyloxy]propyl}pyrrolidine, 1-{2,3-bis[(9Z,12Z)-octadeca-9,12-dienyloxy]propyl}-N,N-dimethylpyrrolidin-3-amine, and 1-[3-[(9Z,12Z)-hexadeca-9,12-dienyloxy]-2-(tetradecyloxy)propyl]pyrrolidine, effectively encapsulate nucleic acids, such as siRNA, with efficiencies from about 50-100%.

In still a further embodiment, functional CaBLES comprising one or more (PEG)-lipid conjugates, one or more non-cationic lipids, and one or more cationic lipids chosen from 1-(2,3-bis((9Z,12Z)-octadeca-9,12-dienyloxy)propyl)pyrrolidine, 1-(2,3-bis((9Z,12Z)-octadeca-9,12-dienyloxy)propyl)-1H-imidazole, 1-(2,3-bis((9Z,12Z)-octadeca-9,12-dienyloxy)propyl)-4-methylpiperazine, 1-(2,3-bis((9Z,12Z)-octadeca-9,12-dienyloxy)propyl)-4-methyl-1,4-diazepane, 1-(2,3-bis((9Z,12Z)-octadeca-9,12-dienyloxy)propyl)-4-phenylpiperazine, 1-(2,3-bis((9Z,12Z)-octadeca-9,12-dienyloxy)propyl)-4-pyridin-2-ylpiperazine, 1-(2,3-bis((9Z,12Z)-octadeca-9,12-dienyloxy)propyl)piperidine, 4-(2,3-bis((9Z,12Z)-octadeca-9,12-dienyloxy)propyl)morpholine, 1-((2R)-2,3-bis((9Z,12Z)-octadeca-9,12-dienyloxy)propyl)pyrrolidine, 1-((2S)-2,3-bis((9Z,12Z)-octadeca-9,12-dienyloxy)propyl)pyrrolidine, 1-(2,3-bis((9Z,12Z)-octadeca-9,12-dienyloxy)propyl)-4-ethylpiperazine, N-(2,3-bis((9Z,12Z)-octadeca-9,12-dienyloxy)propyl)-N-methyl-N-(3-(pyrrolidin-1-ylmethyl)benzyl)amine, N-(2-(4-(2,3-bis((9Z,12Z)-octadeca-9,12-dienyloxy)propyl)piperazin-1-yl)ethyl)-N,N-dimethylamine, 1-((2S)-2,3-bis((9Z,12Z)-octadeca-9,12-dienyloxy)propyl)-4-methylpiperazine, 1-((2R)-2,3-bis((9Z,12Z)-octadeca-9,12-dienyloxy)propyl)-4-methylpiperazine, 1-(2,3-bis((9Z,12Z)-octadeca-9,12-dienyloxy)propyl)-4-(2-pyrrolidin-1-ylethyl)piperazine, 2-(4-(2,3-bis((9Z,12Z)-octadeca-9,12-dienyloxy)propyl)piperazin-1-yl)pyrimidine, 1-(2,3-bis((9Z,12Z)-octadeca-9,12-dienyloxy)propyl)-N,N-diethylpyrrolidin-3-amine, 1-((9Z,12Z)-octadeca-9,12-dienyloxy)-3-pyrrolidin-1-ylpropan-2-ol, 2-[(9Z,12Z)-octadeca-9,12-dienoyloxy]-1-(pyrrolidin-1-ylmethyl)ethyl (9Z,12Z)-octadeca-9,12-dienoate, 2-[(9Z,12Z)-octadeca-9,12-dienyloxy]-1-(pyrrolidin-1-ylmethyl)ethyl (9Z,12Z)-octadeca-9,12-dienoate, 1-({2-[(8Z,11Z)-heptadeca-8,11-dienyl]-2-[(9Z,12Z)-octadeca-9,12-dienyl]-1,3-dioxolan-4-yl}methyl)pyrrolidine, 1-{2,3-bis[(5Z,8Z,11Z,14Z)-icosa-5,8,11,14-tetraenyloxy]propyl}pyrrolidine, 1-{3-[(5Z,8Z,11Z,14Z)-icosa-5,8,11,14-tetraenyloxy]-2-[(9Z,12Z)-octadeca-9,12-dienyloxy]propyl}pyrrolidine, 1-{2,3-bis[(9E,12E)-octadeca-9,12-dienyloxy]propyl}pyrrolidine, 1-{2-[(9E,12E)-octadeca-9,12-dienyloxy]-3-[(9Z,12Z)-octadeca-9,12-dienyloxy]propyl}pyrrolidine, 1-[2,3-bis(tetradecyloxy)propyl]pyrrolidine, 1-[2,3-bis(octadecyloxy)propyl]pyrrolidine, 1-{2,3-bis[(9Z)-octadec-9-enyloxy]propyl}pyrrolidine, 1-[2,3-bis(dodecyloxy)propyl]pyrrolidine, 1-{2,3-bis[(9Z,12Z)-octadeca-9,12-dienyloxy]propyl}pyrrolidin-3-ol, 1-{3-[(9Z,12Z)-hexadeca-9,12-dienyloxy]-2-[(9Z)-octadec-9-enyloxy]propyl}pyrrolidine, 1-{2,3-bis[(9Z,12Z)-octadeca-9,12-dienyloxy]propyl}-N,N-dimethylpyrrolidin-3-amine, and 1-[3-[(9Z,12Z)-hexadeca-9,12-dienyloxy]-2-(tetradecyloxy)propyl]pyrrolidine, effectively encapsulate nucleic acids, such as siRNA, with efficiencies from about 80-100%.

A further embodiment pertains to examples of non-cationic lipids that are useful for the practice of this invention which include, but are not limited to, cholesterol, cholesterol sulfate, ceramide, sphingomyelin, lecithin, sphingomyelin, egg sphingomyelin, milk sphingomyelin; egg phosphatidylcholine, hydrogenated egg phosphatidylcholine, hydrogenated soybean phosphatidylethanolamine, egg phosphatidylethanolamine, hydrogenated soybean phosphatidylcholine, soybean phosphatidylcholine, 1,2-dilauroyl-sn-glycerol, 1,2-dimyristoyl-sn-glycerol, 1,2-dipalmitoyl-sn-glycerol, 1,2-distearoyl-sn-glycerol, 1,2-dilauroyl-sn-glycero-3-phosphatidic acid, 1,2-dimyristoyl-sn-glycero-3-phosphatidic acid, 1,2-dipalmitoyl-sn-glycero-3-phosphatidic acid, 1,2-distearoyl-sn-glycero-3-phosphatidic acid, 1,2-diarachidoyl-sn-glycero-3-phosphocholine, 1,2-dilauroyl-sn-glycero-3-phosphocholine, 1,2-dimyristoyl-sn-glycero-3-phosphocholine, dioleoylphosphatidylcholine, 1,2-dierucoyl-sn-glycero-3-phosphocholine, 1-myristoyl-2-palmitoyl-sn-glycero-3-phosphocholine, 1-myristoyl-2-stearoyl-sn-glycero-3-phosphocholine, 1-palmitoyl-2-myristoyl-sn-glycero-3-phosphocholine, 1-palmitoyl-2-stearoyl-sn-glycero-3-phosphocholine, 1-stearoyl-2-myristoyl-sn-glycero-3-phosphocholine, 1-stearoyl-2-palmitoyl-sn-glycero-3-phosphocholine, 1-myristoyl-2-oleoyl-sn-glycero-3-phosphocholine, 1-palmitoyl-2-oleoyl-sn-glycero-3-phosphocholine; 1-stearoyl-2-oleoyl-sn-glycero-3-phosphocholine, 1-myristoyl-2-lyso-sn-glycero-3-phosphocholine, 1-palmitoyl-2-lyso-sn-glycero-3-phosphocholine, 1-stearoyl-2-lyso-sn-glycero-3-phosphocholine, 1,2-dipalmitoyl-sn-glycero-O-ethyl-3-phosphocholine, 1,2-dipalmitoyl-sn-glycero-3-phosphocholine; 1,2-distearoyl-sn-glycero-3-phosphocholine; 1-palmitoyl-2-linoleoyl-sn-glycero-3-phosphocholine, dioleoylphosphatidylethanolamine, palmitoyloleoyl-phosphatidylethanolamine, dioleoylphosphatidylglycerol,1,2-dilauroyl-sn-glycero-3-phosphoethanolamine, 1,2-dimyristoyl-sn-glycero-3-phosphoethanolamine, 1,2-dipalmitoyl-sn-glycero-3-phosphoethanolamine, 1,2-distearoyl-sn-glycero-3-phosphoethanolamine, 1,2-dioleoyl-sn-glycero-3-phosphoethanolamine, 1,2-dilauroyl-sn-glycero-3-phosphoglycerol, 1,2-dimyristoyl-sn-glycero-3-phosphoglycerol, 1,2-dimyristoyl-sn-glycero-3-phospho-sn-1-glycerol, 1,2-dipalmitoyl-sn-glycero-3-phosphoglycerol, 1,2-distearoyl-sn-glycero-3-phosphoglycero, 1,2-distearoyl-sn-glycero-3-phospho-sn-1-glycerol, 1-palmitoyl-2-oleoyl-sn-glycero-3-phosphoglycerol, 1-palmitoyl-2-oleoyl-sn-glycero-3-phosphoglycerol, 1,2-dipalmitoyl-sn-glycero-3-phospho-L-serine, 1,2-dimyristoyl-sn-glycero-3-phospho-L-serine, 1,2-dipalmitoyl-sn-glycero-3-phospho-L-serine, 1,2-distearoyl-sn-glycero-3-phospho-L-serine, 1,2-dioleoyl-sn-glycero-3-phospho-L-serine, and 1-palmitoyl-2-oleoyl-sn-glycero-3-phospho-L-serine or a mixture thereof.

A further embodiment pertains to examples of PEG-lipid conjugates that are useful for the practice of this invention which include, but are not limited to, 2-(tetradecyloxy)-1-((tetradecyloxy)methyl)ethyl 3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hexatetracontaoxanonatriacontahect-1-ylcarbamate, 2-(hexadecyloxy)-1-((hexadecyloxy)methyl)ethyl 3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hexatetracontaoxanonatriacontahect-1-ylcarbamate, 2-(octadecyloxy)-1-((octadecyloxy)methyl)ethyl 3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hexatetracontaoxanonatriacontahect-1-ylcarbamate, 2-2,5,8,11,14,17,20,23,26,29,32,35,38,41,44,47,50,53,56,59,62,65,68,71,74,77,80,83,86,89,92,94,97,100,103,106,109,112,115,118,121,124,127,130,133,136-hexatetracontaoxaoctatriacontahectanamidopropane-1,3-diyl ditetradecanoate, 2-2,5,8,11,14,17,20,23,26,29,32,35,38,41,44,47,50,53,56,59,62,65,68,71,74,77,80,83,86,89,92,94,97,100,103,106,109,112,115,118,121,124,127,130,133,136-hexatetracontaoxaoctatriacontahectanamidopropane-1,3-diyl dipalmitate, 2-2,5,8,11,14,17,20,23,26,29,32,35,38,41,44,47,50,53,56,59,62,65,68,71,74,77,80,83,86,89,92,94,97,100,103,106,109,112,115,118,121,124,127,130,133,136-hexatetracontaoxaoctatriacontahectanamidopropane-1,3-diyl distearate, N-(2-(hexadecyloxy)-1-((hexadecyloxy)methyl)ethyl)-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide, N-(2-(tetradecyloxy)-1-((tetradecyloxy)methyl)ethyl)-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide,N-(2-(octadecyloxy)-1-((octadecyloxy)methyl)ethyl)-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide, 6-oxo-2-(tetradecanoyloxy)-8,11,14,17,20,23,26,29,32,35,38,41,44,47,50,53,56,59,62,65,68,71,74,77,80,83,86,89,92,95,98,101,104,107,110,113,116,119,122,125,128,131,134,137,140,143-hexatetracontaoxa-5-azatetratetracontahect-1-yl myristate, N-[3,4-bis(tetradecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide, N-[3,4-bis(hexadecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide, N-[3,4-bis(octadecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide, 3,7,11,15,19,23,27,31,35,39,43,47,51,55,59,63,67,71,75,79,83,87,91,95,99,103,107,111,115,119,123,127,131,135,139,143,147,151,155,159,163,167,171,175,179,182-hexatetracontaoxatrioctacontahect-1-yl 3,4-bis(tetradecyloxy)butylcarbamate, 3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hexatetracontaoxanonatriacontahect-1-yl 3,4-bis(hexadecyloxy)butylcarbamate, 3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hexatetracontaoxanonatriacontahect-1-yl 3,4-bis(octadecyloxy)butylcarbamate, N-[3,4-bis(hexadecyloxy)butyl]-N′-3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hexatetracontaoxanonatriacontahect-1-ylsuccinamide, 6-oxo-2-(tetradecanoyloxy)-7,10,13,16,19,22,25,28,31,34,37,40,43,46,49,52,55,58,61,64,67,70,73,76,79,82,85,88,91,94,97,100,103,106,109,112,115,118,121,124,127,130,133,136,139,142,145-heptatetracontaoxa-5-azahexatetracontahect-1-yl myristate, 6-oxo-2-(palmitoyloxy)-7,10,13,16,19,22,25,28,31,34,37,40,43,46,49,52,55,58,61,64,67,70,73,76,79,82,85,88,91,94,97,100,103,106,109,112,115,118,121,124,127,130,133,136,139,142,145-heptatetracontaoxa-5-azahexatetracontahect-1-yl palmitate, 3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hexatetracontaoxanonatriacontahect-1-yl 4-{[3,4-bis(hexadecyloxy)butyl]amino}-4-oxobutanoate, 6-oxo-2-(palmitoyloxy)-8,11,14,17,20,23,26,29,32,35,38,41,44,47,50,53,56,59,62,65,68,71,74,77,80,83,86,89,92,95,98,101,104,107,110,113,116,119,122,125,128,131,134,137,140,143-hexatetracontaoxa-5-azatetratetracontahect-1-yl palmitate, 1,2-distearoyl-sn-glycerol-methoxypolyethyleneglycol-750,1,2-dimyristoyl-sn-glycerol-methoxypolyethyleneglycol-750,1,2-dipalmitoyl-sn-glycerol-methoxypolyethyleneglycol-750, poly(oxy-1,2-ethanediyl)-2000-α-(3)-cholest-5-en-3-yl-omega-hydroxy, 1,2-dipalmitoyl-sn-glycerol-methoxypolyethyleneglycol-5000, poly(oxy-1,2-ethanediyl)-5000-α-(3)-cholest-5-en-3-yl-omega-hydroxy, (2S,3R,E)-3-hydroxy-2-stearamidooctadec-4-enyl polyethyleneglycol-2000 methyl ether succinate, (2S,3R,E)-3-hydroxy-2-icosanamidooctadec-4-enyl polyethyleneglycol-2000 methyl ether succinate N-(2,3-dimyristyloxypropyl)carbamate polyethyleneglycol-2000 methyl ether, N-(carbonylmethoxypolyethyleneglycol-750)-1,2-dimyristoyl-sn-glycero-phosphatidylethanolamine, N-(carbonyl-methoxypolyethyleneglycol-750)-1,2-distearoyl-sn-glycero-3-phosphoethanolamine, N-(carbonyl-methoxypolyethyleneglycol-750)-1,2-dipalmitoyl-sn-glycero-3-phosphoethanolamine, N-(carbonyl-methoxypolyethyleneglycol-2000)-1,2-dimyristoyl-sn-glycero-3-phosphoethanolamine, N-(carbonyl-methoxypolyethyleneglycol-2000)-1,2-dipalmitoyl-sn-glycero-3-phosphoethanolamine, N-(carbonyl-methoxypolyethyleneglycol-2000)-1,2-distearoyl-sn-glycero-3-phosphoethanolamine, N-(carbonyl-methoxypolyethyleneglycol-2000)-dioleoyl-phosphatidylethanolamine, 1,2-distearoyl-sn-glycerol-methoxypolyethyleneglycol-2000,1,2-dimyristoyl-sn-glycerol-methoxypolyethyleneglycol-2000,1,2-dipalmitoyl-sn-glycerol-methoxypolyethyleneglycol-2000, mPEG-2000-cholesterol, octanoyl-mPEG-2000-ceramide, palmitoyl-mPEG-2000-ceramide, N-(carbonyl-methoxypolyethyleneglycol-5000)-1,2-dimyristoyl-sn-glycero-3-phosphoethanolamine, N-(carbonyl-methoxypolyethyleneglycol-5000)-1,2-dipalmitoyl-sn-glycero-3-phosphoethanolamine, N-(carbonyl-methoxypolyethyleneglycol-5000)-1,2-distearoyl-sn-glycero-3-phosphoethanolamine, 1,2-dimyristoyl-sn-glycerol-methoxypolyethyleneglycol-5000,1,2-dipalmitoyl-sn-glycerol-methoxypolyethyleneglycol-5000,1,2-distearoyl-sn-glycerol-methoxypolyethyleneglycol-5000, mPEG-5000-cholesterol, octanoyl-mPEG-5000-ceramide, palmitoyl-mPEG-5000-ceramide and mixtures thereof.

PEG-lipid conjugates are described in, e.g., U.S. App. No. 61/095,748, which was filed on Sep. 10, 2008 and is incorporated herein by reference.

PEG-lipid conjugates are described in, e.g., U.S. App. No. 61/095,769, which was filed on Sep. 10, 2008 and is incorporated herein by reference.

A still further embodiment pertains to combinations of polyethylene glycol (PEG)-lipid conjugates which are useful for the practice of this invention, wherein two PEG-lipid conjugates are chosen from N-(carbonyl-methoxypolyethyleneglycol-2000)-1,2-dimyristoyl-sn-glycero-3-phosphoethanolamine, N-(carbonyl-methoxypolyethyleneglycol-2000)-1,2-dipalmitoyl-sn-glycero-3-phosphoethanolamine, N-(carbonyl-methoxypolyethyleneglycol-2000)-1,2-distearoyl-sn-glycero-3-phosphoethanolamine, 1,2-distearoyl-sn-glycerol-methoxypolyethyleneglycol-2000,1,2-dimyristoyl-sn-glycerol-methoxypolyethyleneglycol-2000,1,2-dipalmitoyl-sn-glycerol-methoxypolyethyleneglycol-2000, and N-[3,4-bis(hexadecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide.

A still further embodiment pertains to combinations of polyethylene glycol (PEG)-lipid conjugates which are useful for the practice of this invention, wherein at least one of the PEG-lipid conjugates is chosen from N-(carbonyl-methoxypolyethyleneglycol-2000)-1,2-dimyristoyl-sn-glycero-3-phosphoethanolamine, N-(carbonyl-methoxypolyethyleneglycol-2000)-1,2-dipalmitoyl-sn-glycero-3-phosphoethanolamine, N-(carbonyl-methoxypolyethyleneglycol-2000)-1,2-distearoyl-sn-glycero-3-phosphoethanolamine, 1,2-distearoyl-sn-glycerol-methoxypolyethyleneglycol-2000,1,2-dimyristoyl-sn-glycerol-methoxypolyethyleneglycol-2000,1,2-dipalmitoyl-sn-glycerol-methoxypolyethyleneglycol-2000, and N-[3,4-bis(hexadecyloxy)butyl]2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide.

A still further embodiment pertains to combinations of polyethylene glycol (PEG)-lipid conjugates which are useful for the practice of this invention and include 1,2-dimyristoyl-sn-glycerol-methoxypolyethyleneglycol-2000 and N-(carbonyl-methoxypolyethyleneglycol-2000)-1,2-distearoyl-sn-glycero-3-phosphoethanolamine, 1,2-distearoyl-sn-glycerol-methoxypolyethyleneglycol-2000 and N-(carbonyl-methoxypolyethyleneglycol-2000)-1,2-dimyristoyl-sn-glycero-3-phosphoethanolamine, N-[3,4-bis(hexadecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide and N-(carbonyl-methoxypolyethyleneglycol-2000)-1,2-distearoyl-sn-glycero-3-phosphoethanolamine, N-(carbonyl-methoxypolyethyleneglycol-2000)-1,2-distearoyl-sn-glycero-3-phosphoethanolamine and N-(carbonyl-methoxypolyethyleneglycol-2000)-1,2-dipalmitoyl-sn-glycero-3-phosphoethanolamine, 1,2-dimyristoyl-sn-glycerol-methoxypolyethyleneglycol-2000 and 1,2-dipalmitoyl-sn-glycerol-methoxypolyethyleneglycol-2000,1,2-distearoyl-sn-glycerol-methoxypolyethyleneglycol-2000 and 1,2-dimyristoyl-sn-glycerol-methoxypolyethyleneglycol-2000; N-(carbonyl-methoxypolyethyleneglycol-2000)-1,2-dimyristoyl-sn-glycero-3-phosphoethanolamine and N-(carbonyl-methoxypolyethyleneglycol-2000)-1,2-distearoyl-sn-glycero-3-phosphoethanolamine, N-(carbonyl-methoxypolyethyleneglycol-2000)-1,2-dimyristoyl-sn-glycero-3-phosphoethanolamine and N-(carbonyl-methoxypolyethyleneglycol-2000)-1,2-dipalmitoyl-sn-glycero-3-phosphoethanolamine, and N-[3,4-bis(hexadecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide and 1,2-distearoyl-sn-glycerol-methoxypolyethyleneglycol-2000.

In still a further embodiment, the cationic lipids of the CaBLES and Lipid-Based Particles comprises about 2 to about 60 weight/weight percent of total lipid in the particle.

In still a further embodiment, the non-cationic lipids of the Cables and Lipid-Based Particles comprises about 5 to about 90 weight/weight percent of total lipid in the particle.

In still a further embodiment, the PEG-lipid conjugates of the CaBLES and Lipid-Based Particles comprises from 0.1 to about 20 weight/weight percent of total lipid in the particle.

### Methods of Treatment and Methods of Making Lipid-Based Particles

Still another embodiment pertains to a method of treating cancer in a mammal comprising administering thereto a Lipid-Based Particle.

Still another embodiment comprises methods of treating cancer in a mammal comprising administering thereto a Lipid-Based Particle comprising one or more cationic lipids having Formula (I)

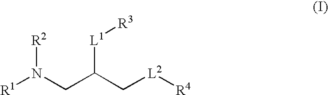

wherein

R¹ and R² are independently H, cycloalkyl, cycloalkenyl or R⁵; or

R¹ and R², taken together with the atoms to which they are attached, are heterocycloalkyl or heteroaryl;

L¹ is O, OC(O) or (O)CO;

L² is O, OC(O) or (O)CO;

one of R³ and R⁴ is H, and the other is C₁₄-C₂₀-alkenyl or C₁₄-C₂₀-alkyl; or

R³ and R⁴ are independently C₁₄-C₂₀-alkenyl or C₁₄-C₂₀-alkyl; or

R³ and R⁴ combine to form CR²⁰R²¹, wherein R²⁰ is H and R²¹ is C₁₄-C₂₀-alkenyl or C₁₄-C₂₀-alkyl; or R²⁰ and R²¹ are the same or are different and are C₁₄-C₂₀-alkenyl, C₁₄-C₂₀-alkyl, or (CH₂O)—C₁₄-C₂₀ alkenyl;

R⁵ is alkyl, which is unsubstituted or substituted with one or more R⁶, OR⁶, SR⁶S(O)R⁶, SO₂R⁶, C(O)R⁶, CO(O)R⁶, OC(O)R⁶, OC(O)OR⁶, NH₂, NHR⁶, N(R⁶)₂, NHC(O)R⁶, NR⁶C(O)R⁶, NHS(O)₂R⁶, N R⁶S(O)₂R⁶, NHC(O)OR⁶, NR⁶C(O)OR⁶, NHC(O)NH₂, NHC(O)NHR⁶, NHC(O)N(R⁶)₂, NR⁶C(O)NHR⁶, NR⁶C(O)N(R⁶)₂, C(O)NH₂, C(O)NHR⁶, C(O)N(R⁶)₂, C(O)NHOH, C(O)NHOR⁶, C(O)NHSO₂R⁶, C(O)NR⁶SO₂R⁶, SO₂NH₂, SO₂NHR⁶, SO₂N(R⁶)₂, C(O)H, C(O)OH, C(N)NH₂, C(N)NHR⁶, C(N)N(R⁶)₂, CNOH, CNOCH₃, OH, (O), CN, N₃, NO₂, CF₃, CF₂CF₃, OCF₃, OCF₂CF₃, F, Cl, Br or I;

R⁶ is R⁷, R⁸, R⁹, or R¹⁰

R⁷ is phenyl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R⁸ is heteroaryl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R⁹ is cycloalkyl, cycloalkenyl, heterocycloalkyl or heterocycloalkenyl, each of which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹⁰ is alkyl, alkenyl or alkynyl;

wherein each foregoing cyclic moiety is independently unsubstituted or substituted with one or more R¹¹, OR¹¹, SR¹¹, S(O)R¹¹, SO₂R¹¹, C(O)R¹¹, CO(O)R¹¹, OC(O)R¹¹, OC(O)OR¹¹, NH₂, NHR¹¹, N(R¹¹)₂, NHC(O)R¹¹, NR¹¹C(O)R¹¹, NHS(O)₂R¹¹, N R¹¹S(O)₂R¹¹, NHC(O)OR¹¹, NR¹¹C(O)OR¹¹, NHC(O)NH₂, NHC(O)NHR¹¹, NHC(O)N(R¹¹)₂, NR¹¹C(O)NHR¹¹, NR¹¹C(O)N(R¹¹)₂, C(O)NH₂, C(O)NHR¹¹, C(O)N(R¹¹)₂, C(O)NHOH, C(O)NHOR¹¹, C(O)NHSO₂R¹¹, C(O)NR¹¹SO₂R¹¹, SO₂NH₂, SO₂NHR¹¹, SO₂N(R¹¹)₂, C(O)H, C(O)OH, C(N)NH₂, C(N)NHR¹¹, C(N)N(R¹¹)₂, CNOH, CNOCH₃, OH, (O), CN, N₃, NO₂, CF₃, CF₂CF₃, OCF₃, OCF₂CF₃, F, Cl, Br or I;

R¹¹ is R¹², R¹³, R¹⁴, or R¹⁵

R¹² is phenyl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹³ is heteroaryl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹⁴ is cycloalkyl, cycloalkenyl, heterocycloalkyl or heterocycloalkenyl, each of which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹⁵ is alkyl, alkenyl or alkynyl, each of which is unsubstituted or substituted with one or two of independently selected R¹⁶, OR¹⁶, SR¹⁶, S(O)₂R¹⁶, C(O)OH, NH₂, NHR¹⁶N(R¹⁶)₂, C(O)R¹⁶,C(O)NH₂, C(O)NHR¹⁶, C(O)N(R¹⁶)₂, NHC(O)R¹⁶, NR¹⁶C(O)R¹⁶, NHC(O)OR¹⁶NR¹⁶C(O)OR¹⁶, OH, F, Cl, Br or I;

R¹⁶ is alkyl, alkenyl, alkynyl, or R¹⁷;

R¹⁷ is phenyl, heteroaryl, cycloalkyl, cycloalkenyl, heterocycloalkyl or heterocycloalkenyl, each of which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

wherein R¹², R¹³, R¹⁴, and R¹⁷ are independently unsubstituted or substituted with one or more R¹⁸,OR¹⁸, SR¹⁸,S(O)R¹⁸, SO₂R¹⁸, C(O)R¹⁸, CO(O)R¹⁸, OC(O)R⁸OC(O)OR¹⁸, NH₂, NHR¹⁸, N(R¹⁸)₂, NHC(O)R¹⁸, NR¹C(O)R¹⁸, NHS(O)₂R¹⁸, NR¹⁸S(O)₂R¹⁸, NHC(O)OR¹⁸, NR¹⁸C(O)OR¹⁸, NHC(O)NH₂, NHC(O)NHR¹⁸, NHC(O)N(R¹⁸)₂, NR¹⁸C(O)NHR¹⁸, NR¹⁸C(O)N(R¹⁸)₂, C(O)NH₂, C(O)NHR¹⁸OC(O)N(R¹⁸)₂, C(O)NHOH, C(O)NHOR¹⁸, C(O)NHSO₂R¹⁸, C(O)NR¹⁸SO₂R¹⁸, SO₂NH₂, SO₂NHR¹⁸, SO₂N(R¹⁸)₂, C(O)H, C(O)OH, C(N)NH₂, C(N)NHR¹⁸, C(N)N(R¹⁸)₂, CNOH, CNOCH₃, OH, (O), CN, N₃, NO₂, CF₃, CF₂CF₃, OCF₃,OCF₂CF₃, F, Cl, Br or I; and

R¹⁸ is alkyl, alkenyl, alkynyl, phenyl, heteroaryl, cycloalkyl, cycloalkenyl, heterocycloalkyl or heterocycloalkenyl; and

one or more non-cationic lipids, one or more polyethylene glycol-lipid conjugates and one or more therapeutic agents.

A further embodiment pertains to a method of making CaBLES or Lipid-Based

Particles, comprising: (a) mixing the cationic lipid(s), the non-cationic lipid(s) and the PEG-lipid conjugate(s); (b) adding the mixture of step (a) to one or more therapeutic agents; and (c) separating and purifying resulting suspension of step (b).

A further embodiment pertains to a method of making Lipid-Based Particles wherein the therapeutic agent is warmed to about 60° C. prior to the addition of the mixture of step (a) via needle injection.

### Pharmaceutical Compositions and Methods of Administration

Therapeutically effective amounts of Lipid-Based Particles of this invention depend on recipient of treatment, disease treated and severity thereof, composition comprising it, time of administration, route of administration, duration of treatment, potency, rate of clearance and whether or not another drug is co-administered. The amount of Lipid-Based Particles of this invention used to make compositions to be administered daily to a patient in a single dose or in divided doses is from about 0.001 to about 200 mg/kg body weight. Single dose compositions contain these amounts or a combination of submultiples thereof.

One embodiment pertains to a pharmaceutical composition comprising one or more (PEG)-lipid conjugates, one or more non-cationic lipids, one or more cationic lipids of Formula 1, one or more therapeutic agents, and a pharmaceutically acceptable excipient.

Lipid-Based Particles of this invention may be administered, for example, bucally, ophthalmically, orally, osmotically, parenterally (intramuscularly, intraperintoneally intrasternally, intravenously, subcutaneously), rectally, topically, transdermally, vaginally and intraarterially as well as by intraarticular injection, infusion, and placement in the body, such as, for example, the vasculature.

Lipid-Based Particles may be administered with or without an excipient. Excipients include, but are not limited to, encapsulators and additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, mixtures thereof and the like.

Excipients for preparation of compositions comprising Lipid-Based Particles to be administered orally include, but are not limited to, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl celluose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, mixtures thereof and the like. Excipients for preparation of compositions comprising a compound having formula (I) to be administered ophthalmically or orally include, but are not limited to, 1,3-butylene glycol, castor oil, corn oil, cottonseed oil, ethanol, fatty acid esters of sorbitan, germ oil, groundnut oil, glycerol, isopropanol, olive oil, polyethylene glycols, propylene glycol, sesame oil, water, mixtures thereof and the like. Excipients for preparation of compositions comprising a compound having formula (I) to be administered osmotically include, but are not limited to, chlorofluorohydrocarbons, ethanol, water, mixtures thereof and the like. Excipients for preparation of compositions comprising a compound having formula (I) to be administered parenterally include, but are not limited to, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water, mixtures thereof and the like. Excipients for preparation of compositions comprising a compound having formula (I) to be administered rectally or vaginally include, but are not limited to, cocoa butter, polyethylene glycol, wax, mixtures thereof and the like.

The pharmaceutical composition and the method of the present invention may further comprise other therapeutically active compounds as noted herein which are usually applied in the treatment of the above-mentioned pathological conditions.

### Combination Therapy

The present invention further provides methods of using a compound, formulation, or composition of the invention in combination with one or more additional active agents.

Lipid-Based Particles are expected to be useful when used with: alkylating agents, angiogenesis inhibitors, antibodies, antimetabolites, antimitotics, antiproliferatives, aurora kinase inhibitors, apoptosis promoters (for example, Bcl-xL, Bcl-w and Bfl-1) inhibitors, Bcr-Abl kinase inhibitors, BiTE (Bi-Specific T cell Engager) antibodies, biologic response modifiers, cyclin-dependent kinase inhibitors, cell cycle inhibitors, cyclooxygenase-2 inhibitors, DVD's, leukemia viral oncogene homolog (ErbB2) receptor inhibitors, growth factor inhibitors, heat shock protein (HSP)-90 inhibitors, histone deacetylase (HDAC) inhibitors, hormonal therapies, immunologicals, inhibitors of apoptosis proteins (IAP's) intercalating antibiotics, kinase inhibitors, mammalian target of rapamycin inhibitors, microRNA's mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, proteosome inhibitors, purine analogs, pyrimidine analogs, receptor tyrosine kinase inhibitors, retinoids/deltoids plant alkaloids, small inhibitory ribonucleic acids (siRNA's), topoisomerase inhibitors, combinations thereof and the like.

A BiTE antibody is a bi-specific antibody that directs T-cells to attach cancer cells by simultaneously binding the two cells. The T-cell then attacks the target cancer cell. Exemplary BiTE antibodies include adecatumumab (Micromet MT201), blinatumomab (Micromet MT103) and the like.

SiRNA's are molecules having endogenous RNA bases or chemically modified nucleotides. The modifications shall not abolish cellular activity, but rather impart increased stability and/or increased cellular potency. Examples of chemical modifications include phosphorothioate groups, 2′-deoxynucleotide, 2′-OCH₃-containing ribonucleotides, 2′-F-ribonucleotides, 2′-methoxyethyl ribonucleotides or a combination thereof. The siRNA can have varying lengths (10-200 bps) and structures (hairpins, single/double strands, bulges, nicks/gaps, mismatches) and processed in the cell to provide active gene silencing. In certain embodiments, a double-stranded siRNA (dsRNA) can have the same number of nucleotides on each strand (blunt ends) or asymmetric ends (overhangs). The overhang of 1-2 nucleotides can be present on the sense and/or the antisense strand, as well as present on the 5′- and/or the 3′-ends of a given strand.

Multivalent binding proteins are binding proteins comprising two or more antigen binding sites. The multivalent binding protein is preferably engineered to have the three or more antigen binding sites and is generally not a naturally occurring antibody. The term “multispecific binding protein” means a binding protein capable of binding two or more related or unrelated targets. Dual variable domain (DVD) binding proteins are tetravalent or multivalent binding proteins binding proteins comprising two or more antigen binding sites. Such DVDs may be monospecific, i.e., capable of binding one antigen or multispecific, i.e., capable of binding two or more antigens. DVD binding proteins comprising two heavy chain DVD polypeptides and two light chain DVD polypeptides are referred to as DVD Ig. Each half of a DVD Ig comprises a heavy chain DVD polypeptide, a light chain DVD polypeptide, and two antigen binding sites. Each binding site comprises a heavy chain variable domain and a light chain variable domain with a total of 6 CDRs involved in antigen binding per antigen binding site.

Alkylating agents include altretamine, AMD-473, AP-5280, apaziquone, bendamustine, brostallicin, busulfan, carboquone, carmustine (BCNU), chlorambucil, CLORETAZINE® (laromustine, VNP 40101M), cyclophosphamide, decarbazine, estramustine, fotemustine, glufosfamide, ifosfamide, KW-2170, lomustine (CCNU), mafosfamide, melphalan, mitobronitol, mitolactol, nimustine, nitrogen mustard N-oxide, ranimustine, temozolomide, thiotepa, TREANDA® (bendamustine), treosulfan, rofosfamide and the like.

Angiogenesis inhibitors include endothelial-specific receptor tyrosine kinase (Tie-2) inhibitors, epidermal growth factor receptor (EGFR) inhibitors, insulin growth factor-2 receptor (IGFR-2) inhibitors, matrix metalloproteinase-2 (MMP-2) inhibitors, matrix metalloproteinase-9 (MMP-9) inhibitors, platelet-derived growth factor receptor (PDGFR) inhibitors, thrombospondin analogs, vascular endothelial growth factor receptor tyrosine kinase (VEGFR) inhibitors and the like.

Antimetabolites include ALIMTA® (metrexed disodium, LY231514, MTA), 5-azacitidine, XELODA® (capecitabine), carmofur, LEUSTAT® (cladribine), clofarabine, cytarabine, cytarabine ocfosfate, cytosine arabinoside, decitabine, deferoxamine, doxifluridine, eflomithine, EICAR (5-ethynyl-1-β-D-ribofuranosylimidazole-4-carboxamide), enocitabine, ethnylcytidine, fludarabine, 5-fluorouracil alone or in combination with leucovorin, GEMZAR® (gemcitabine), hydroxyurea, ALKERAN® (melphalan), mercaptopurine, 6-mercaptopurine riboside, methotrexate, mycophenolic acid, nelarabine, nolatrexed, ocfosfate, pelitrexol, pentostatin, raltitrexed, Ribavirin, triapine, trimetrexate, S-1, tiazofurin, tegafur, TS-1, vidarabine, UFT and the like.

Bcl-2 proteins inhibitors include AT-101 ((−)gossypol), GENASENSE® (G3139 or oblimersen (Bcl-2-targeting antisense oligonucleotide)), IPI-194, IPI-565, N-(4-(4-((4′-chloro(1,1′-biphenyl)-2-yl)methyl)piperazin-1-yl)benzoyl)-4-(((1R)-3-(dimethylamino)-1-((phenylsulfanyl)methyl)propyl)amino)-3-nitrobenzenesulfonamide) (ABT-737), N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide (ABT-263), GX-070 (obatoclax) and the like.

Bcr-Abl kinase inhibitors include DASATINIB® (BMS-354825), GLEEVEC® (imatinib) and the like.

CDK inhibitors include AZD-5438, BMI-1040, BMS-032, BMS-387, CVT-2584, flavopyridol, GPC-286199, MCS-5A, PD0332991, PHA-690509, seliciclib (CYC-202, R-roscovitine), ZK-304709 and the like.

COX-2 inhibitors include ABT-963, ARCOXIA® (etoricoxib), BEXTRA® (valdecoxib), BMS347070, CELEBREX® (celecoxib), COX-189 (lumiracoxib), CT-3, DERAMAXX® (deracoxib), JTE-522,4-methyl-2-(3,4-dimethylphenyl)-1-(4-sulfamoylphenyl-1H-pyrrole), MK-663 (etoricoxib), NS-398, parecoxib, RS-57067, SC-58125, SD-8381, SVT-2016, S-2474, T-614, VIOXX® (rofecoxib) and the like.

EGFR inhibitors include ABX-EGF, anti-EGFR immunoliposomes, EGF-vaccine, EMD-7200, ERBITUX® (cetuximab), HR3, IgA antibodies, IRESSA® (gefitinib), TARCEVA® (erlotinib or OSI-774), TP-38, EGFR fusion protein, TYKERB® (lapatinib) and the like.

ErbB2 receptor inhibitors include CP-724-714, CI-1033 (canertinib), HERCEPTIN® (trastuzumab), TYKERB® (lapatinib), OMNITARG® (2C4, petuzumab), TAK-165, GW-572016 (ionafarnib), GW-282974, EKB-569, PI-166, dHER2 (HER2 vaccine), APC-8024 (HER-2 vaccine), anti-HER/2neu bispecific antibody, B7.her2IgG3, AS HER2 trifunctional bispecfic antibodies, mAB AR-209, mAB 2B-1 and the like.

Histone deacetylase inhibitors include depsipeptide, LAQ-824, MS-275, trapoxin, suberoylanilide hydroxamic acid (SAHA), TSA, valproic acid and the like.

HSP-90 inhibitors include 17-AAG-nab, 17-AAG, CNF-101, CNF-1010, CNF-2024, 17-DMAG, geldanamycin, IPI-504, KOS-953, MYCOGRAB® (human recombinant antibody to HSP-90), NCS-683664, PU24FCl, PU-3, radicicol, SNX-2112, STA-9090 VER49009 and the like.

Inhibitors of apoptosis proteins include ApoMab (a fully human affinity-matured IgG1 monoclonal antibody), antibodies that target TRAIL or death receptors (e.g., pro-apoptotic receptor agonists DR4 and DR5), conatumumab, ETR2-ST01, GDC0145, (lexatumumab), HGS-1029, LBY-135, PRO-1762 and tratuzumab.

MEK inhibitors include ARRY-142886, ARRY-438162 PD-325901, PD-98059 and the like.

mTOR inhibitors include AP-23573, CCI-779, everolimus, RAD-001, rapamycin, temsirolimus and the like.

Non-steroidal anti-inflammatory drugs include AMIGESIC® (salsalate), DOLOBID® (diflunisal), MOTRIN® (ibuprofen), ORUDIS® (ketoprofen), RELAFEN® (nabumetone), FELDENE® (piroxicam), ibuprofen cream, ALEVE® (naproxen) and NAPROSYN® (naproxen), VOLTAREN® (diclofenac), INDOCIN® (indomethacin), CLINORIL® (sulindac), TOLECTIN® (tolmetin), LODINE® (etodolac), TORADOL® (ketorolac), DAYPRO® (oxaprozin) and the like.

PDGFR inhibitors include C-451, CP-673, CP-868596 and the like.

Platinum chemotherapeutics include cisplatin, ELOXATIN® (oxaliplatin) eptaplatin, lobaplatin, nedaplatin, PARAPLATIN® (carboplatin), satraplatin and the like.

Polo-like kinase inhibitors include BI-2536 and the like.

Thrombospondin analogs include ABT-510, ABT-567, TSP-1 and the like.

VEGFR inhibitors include AVASTIN® (bevacizumab), ABT-869, AEE-788, ANGIOZYME™ (a ribozyme that inhibits angiogenesis (Ribozyme Pharmaceuticals (Boulder, Colo.) and Chiron, (Emeryville, Calif.)), axitinib (AG-13736), AZD-2171, CP-547,632, IM-862, MACUGEN (pegaptamib), NEXAVAR® (sorafenib, BAY43-9006), pazopanib (GW-786034), vatalanib (PTK-787, ZK-222584), SUTENT® (sunitinib, SU-11248), VEGF trap, ZACTIMA™ (vandetanib, ZD-6474) and the like.

Antibiotics include intercalating antibiotics aclarubicin, actinomycin D, amrubicin, annamycin, adriamycin, BLENOXANE® (bleomycin), daunorubicin, CAELYX® or MYOCET® (liposomal doxorubicin), elsamitrucin, epirbucin, glarbuicin, ZAVEDOS® (idarubicin), mitomycin C, nemorubicin, neocarzinostatin, peplomycin, pirarubicin, rebeccamycin, stimalamer, streptozocin, VALSTAR® (valrubicin), zinostatin and the like.

Topoisomerase inhibitors include aclarubicin, 9-aminocamptothecin, amonafide, amsacrine, becatecarin, belotecan, BN-80915, CAMPTOSAR® (irinotecan hydrochloride), camptothecin, CARDIOXANE® (dexrazoxine), diflomotecan, edotecarin, ELLENCE® or PHARMORUBICIN® (epirubicin), etoposide, exatecan, 10-hydroxycamptothecin, gimatecan, lurtotecan, mitoxantrone, orathecin, pirarbucin, pixantrone, rubitecan, sobuzoxane, SN-38, tafluposide, topotecan and the like.

Antibodies include AVASTIN® (bevacizumab), CD40-specific antibodies, chTNT-1/B, denosumab, ERBITUX® (cetuximab), HUMAX-CD4® (zanolimumab), IGF1R-specific antibodies, lintuzumab, PANOREX® (edrecolomab), RENCAREX® (WX G250), RITUXAN® (rituximab), ticilimumab, trastuzimab and the like.

Hormonal therapies include ARIMIDEX® (anastrozole), AROMASIN® (exemestane), arzoxifene, CASODEX® (bicalutamide), CETROTIDE® (cetrorelix), degarelix, deslorelin, DESOPAN® (trilostane), dexamethasone, DROGENIL®, (flutamide), EVISTA® (raloxifene), AFEMA™ (fadrozole), FARESTON® (toremifene), FASLODEX® (fulvestrant), FEMARA® (letrozole), formestane, glucocorticoids, HECTOROL® (doxercalciferol), RENAGEL® (sevelamer carbonate), lasofoxifene, leuprolide acetate, MEGACE® (megesterol), MIFEPREX® (mifepristone), NILANDRON™ (nilutamide), NOLVADEX® (tamoxifen citrate), PLENAXIS™ (abarelix), prednisone, PROPECIA® (finasteride), rilostane, SUPREFACT® (buserelin), TRELSTAR® (luteinizing hormone releasing hormone (LHRH)), VANTAS® (Histrelin implant), VETORYL® (trilostane or modrastane), ZOLADEX® (fosrelin, goserelin) and the like.

Deltoids and retinoids include seocalcitol (EB1089, CB1093), lexacalcitrol (KH1060), fenretinide, PANRETIN® (aliretinoin), ATRAGEN® (liposomal tretinoin), TARGRETIN® (bexarotene), LGD-1550 and the like.

PARP inhibitors include ABT-888, olaparib, KU-59436, AZD-2281, AG-014699, BSI-201, BGP-15, INO-1001, ONO-2231 and the like.

Plant alkaloids include, but are not limited to, vincristine, vinblastine, vindesine, vinorelbine and the like.

Proteasome inhibitors include VELCADE® (bortezomib), MG132, NPI-0052, PR-171 and the like.

Examples of immunologicals include interferons and other immune-enhancing agents. Interferons include interferon alpha, interferon alpha-2a, interferon alpha-2b, interferon beta, interferon gamma-1a, ACTIMMUNE® (interferon gamma-1b), or interferon gamma-n1, combinations thereof and the like. Other agents include ALFAFERONE®, (IFN-α), BAM-002 (oxidized glutathione), BEROMUN® (tasonermin), BEXXAR® (tositumomab), CAMPATH® (alemtuzumab), CTLA4 (cytotoxic lymphocyte antigen 4), decarbazine, denileukin, epratuzumab, GRANOCYTE® (lenograstim), lentinan, leukocyte alpha interferon, imiquimod, MDX-010 (anti-CTLA-4), melanoma vaccine, mitumomab, molgramostim, MYLOTARG™ (gemtuzumab ozogamicin), NEUPOGEN® (filgrastim), Onco VAC-CL, OVAREX® (oregovomab), pemtumomab (Y-muHMFG1), PROVENGE® (sipuleucel-T), sargaramostim, sizofilan, teceleukin, THERACYS® (Bacillus Calmette-Guerin), ubenimex, VIRULIZIN® (immunotherapeutic, Lorus Pharmaceuticals), Z-100 (Specific Substance of Maruyama (SSM)), WF-10 (Tetrachlorodecaoxide (TCDO)), PROLEUKIN® (aldesleukin), ZADAXIN® (thymalfasin), ZENAPAX® (daclizumab), ZEVALIN® (90Y-Ibritumomab tiuxetan) and the like.

Biological response modifiers are agents that modify defense mechanisms of living organisms or biological responses, such as survival, growth, or differentiation of tissue cells to direct them to have anti-tumor activity and include include krestin, lentinan, sizofuran, picibanil PF-3512676 (CpG-8954), ubenimex and the like.

Pyrimidine analogs include cytarabine (ara C or Arabinoside C), cytosine arabinoside, doxifluridine, FLUDARA® (fludarabine), 5-FU (5-fluorouracil), floxuridine, GEMZAR® (gemcitabine), TOMUDEX® (ratitrexed), TROXATYL™ (triacetyluridine troxacitabine) and the like.

Purine analogs include LANVIS® (thioguanine) and PURI-NETHOL® (mercaptopurine).

Antimitotic agents include batabulin, epothilone D (KOS-862), N-(2-((4-hydroxyphenyl)amino)pyridin-3-yl)-4-methoxybenzenesulfonamide, ixabepilone (BMS 247550), paclitaxel, TAXOTERE® (docetaxel), PNU100940 (109881), patupilone, XRP-9881 (larotaxel), vinflunine, ZK-EPO (synthetic epothilone) and the like.

Compounds of this invention can also be used as radiosensitizeser that enhance the efficacy of radiotherapy. Examples of radiotherapy include external beam radiotherapy, teletherapy, brachtherapy and sealed, unsealed source radiotherapy and the like.

Additionally, compounds having Formula I may be combined with other chemptherapeutic agents such as ABRAXANE™ (ABI-007), ABT-100 (farnesyl transferase inhibitor), ADVEXIN® (Ad5CMV-p53 vaccine), ALTOCOR® or MEVACOR® (lovastatin), AMPLIGEN® (poly I:poly C12U, a synthetic RNA), APTOSYN® (exisulind), AREDIA® (pamidronic acid), arglabin, L-asparaginase, atamestane (1-methyl-3,17-dione-androsta-1,4-diene), AVAGE® (tazarotene), AVE-8062 (combreastatin derivative) BEC2 (mitumomab), cachectin or cachexin (tumor necrosis factor), canvaxin (vaccine), CEAVAC® (cancer vaccine), CELEUK® (celmoleukin), CEPLENE® (histamine dihydrochloride), CERVARIX® (human papillomavirus vaccine), CHOPφ (C: CYTOXAN® (cyclophosphamide); H: ADRIAMYCIN® (hydroxydoxorubicin); O: Vincristine (ONCOVIN®); P: prednisone), CYPATT™ (cyproterone acetate), combrestatin A4P, DAB (3 89)EGF (catalytic and translocation domains of diphtheria toxin fused via a His-Ala linker to human epidermal growth factor) or TransMID-107R™ (diphtheria toxins), dacarbazine, dactinomycin, 5,6-dimethylxanthenone-4-acetic acid (DMXAA), eniluracil, EVIZON™ (squalamine lactate), DIMERICINE® (T4N5 liposome lotion), discodermolide, DX-8951f (exatecan mesylate), enzastaurin, EPO906 (epithilone B), GARDASIL® (quadrivalent human papillomavirus (Types 6, 11, 16, 18) recombinant vaccine), GASTRIMMUNE®, GENASENSE®, GMK (ganglioside conjugate vaccine), GVAX® (prostate cancer vaccine), halofuginone, histerelin, hydroxycarbamide, ibandronic acid, IGN-101, IL-13-PE38, IL-13-PE38QQR (cintredekin besudotox), IL-13-pseudomonas exotoxin, interferon-α, interferon-γ, JUNOVAN™ or MEPACT™ (mifamurtide), lonafamib, 5,10-methylenetetrahydrofolate, miltefosine (hexadecylphosphocholine), NEOVASTAT® (AE-941), NEUTREXIN® (trimetrexate glucuronate), NIPENT® (pentostatin), ONCONASE® (a ribonuclease enzyme), ONCOPHAGE® (melanoma vaccine treatment), ONCOVAX® (IL-2 Vaccine), ORATHECIN™ (rubitecan), OSIDEM® (antibody-based cell drug), OVAREX® MAb (murine monoclonal antibody), paditaxel, PANDIMEX™ (aglycone saponins from ginseng comprising 20 (S)protopanaxadiol (aPPD) and 20 (S)protopanaxatriol (aPPT)), panitumumab, PANVAC®-VF (investigational cancer vaccine), pegaspargase, PEG Interferon A, phenoxodiol, procarbazine, rebimastat, REMOVAB® (catumaxomab), REVLIMID® (lenalidomide), RSR13 (efaproxiral), SOMATULINE® LA (lanreotide), SORIATANE® (acitretin), staurosporine (Streptomyces staurospores), talabostat (PT 100), TARGRETIN® (bexarotene), TAXOPREXIN® (DHA-paclitaxel), TELCYTA® (canfosfamide, TLK286), temilifene, TEMODAR® (temozolomide), tesmilifene, thalidomide, THERATOPE® (STn-KLH), thymitaq (2-amino-3,4-dihydro-6-methyl-4-oxo-5-(4-pyridylthio)quinazoline dihydrochloride), TNFERADE™ (adenovector: DNA carrier containing the gene for tumor necrosis factor-α), TRACLEER® or ZAVESCA® (bosentan), tretinoin (Retin-A), tetrandrine, TRISENOX® (arsenic trioxide), VIRULIZIN®, ukrain (derivative of alkaloids from the greater celandine plant), vitaxin (anti-alphavbeta3 antibody), XCYTRIN® (motexafin gadolinium), XINLAY™ (atrasentan), XYOTAX™ (paclitaxel poliglumex), YONDELIS® (trabectedin), ZD-6126, ZINECARD® (dexrazoxane), ZOMETA® (zolendronic acid), zorubicin and the like.

### Cationic-Based Lipid Encapsulation Systems (CaBLES) and Lipid-Based Particles

CaBLES comprise one or more non-cationic lipids, one or more cationic lipids having Formula I and one or more polyethylene glycol (PEG)-lipid conjugate.

Lipid-Based Particles of the present invention are defined as CaBLES which further comprise one or more therapeutic agent(s). These particles have mean diameter sizes of 50-300 nm, of which 50-250 nm is preferred and 50-200 nm is most preferred. Functional CaBLES effectively encapsulate nucleic acids, (e.g., single stranded or double stranded DNA, single stranded or double stranced RNA, RNAi, siRNA, and the like). Suitable nucleic acids include, but are not limited to, plasmids, antisense oligonucleotides, ribozymes as well as other poly- and oligonucleotides. In preferred embodiments, the nucleic acid encodes a product, e.g., a therapeutic product, of interest. The CaBLES of the present invention can be used to deliver the nucleic acid to a cell (e.g., a cell in a mammal) for, e.g., expression of the nucleic acid or for silencing of a target sequence expressed by the cell.

In some embodiments, the nucleic acid is a siRNA molecule that silences the gene of interest, with efficiencies from about 50-100%, and more preferably between about 80-100%.

In other embodiments, the therapeutic agents that can be delivered with CaBLES include RNA, antisense oligonucleotide, a DNA, a plasmid, a ribosomal RNA (rRNA), a micro RNA (miRNA), transfer RNA (tRNA), a small inhibitory RNA (siRNA), small nuclear RNA (snRNA), chimeric nucleic acids, an antigen, fragments thereof, a protein, a peptide, small-molecules, or mixtures thereof. This invention describes delivery of RNA's such as small inhibitory RNA or microRNA. The siRNA can have varying lengths (10-200 bps) and structures (hairpins, single/double strands, bulges, nicks/gaps, mismatches) and processed in the cell to provide active gene silencing. In certain embodiments of this invention, a double-stranded siRNA (dsRNA) can have the same number of nucleotides on each strand (blunt ends) or asymmetric ends (overhangs). The overhang of 1-2 nucleotides can be present on the sense and/or the antisense strand, as well as present on the 5′- and/or the 3′-ends of a given strand.

Suitable siRNA sequences can be identified using means known in the art (e.g., methods described in Elbashir, et al., Nature 411:494-498 (2001) and Elbashir, et al., EMBO J. 20: 6877-6888 (2001) are combined with rational design rules set forth in Reynolds et al., Nature Biotech. 22(3):326-330 (2004)). Further enhancing, isolating, synthesizing and generating of the siRNA can be done by various methods known in the art, (see, e.g., Elbashir, et al., EMBO J. 20: 6877-6888 (2001); Elbashir, et al., Genes Dev. 15:188 (2001); Nykanen, et al., Cell 107:309 (2001)) or may lack overhangs (i.e., to have blunt ends): and Gubler & Hoffman, Gene 25:263-269 (1983); Sambrook et al., Molecular Cloning, A Laboratory Manual (2nd ed. 1989); Current Protocols in Molecular Biology (Ausubel et al., eds., 1994), as are PCR methods (see U.S. Pat. Nos. 4,683,195 and 4,683,202; PCR Protocols: A Guide to Methods and Applications (Innis et al., eds, 1990)).

Non-cationic lipids have a neutral charge or an anionic charge at physiological pH. A neutral lipid, also known as a “helper lipid,” has no net charge at physiological pH. These lipids can also be zwitterionic.

Polyethylene glycol (PEG)-lipid conjugates are used to minimize particle aggregation in solution, provide increased in vivo serum circulation, and enhance distribution of nanoparticles to organs, tissues, cell types, and tumors of interest. These shielding lipids consist of a lipid portion linked to a “PEG” portion via carbamate, ester, amide, ether, amine, thioether, or dithiol linkages. “PEG” is a polyethylene glycol consisting of repeating C₂H₄O units with an average molecular weight between 500 to 10,000 daltons and may be substituted by alkoxy, acyl, alkyl, or aryl. Additionally, the PEG can be substituted at its terminus with one or more of the following functional groups: hydroxy, methoxy, primary, secondary, or tertiary amine, thiol, thioether, thiopyridyl, dithiol, maleimide, or ester.

In some instances it may be desirable for the CaBLES and/or Lipid Based Particles to target using targeting moieties that are specific to a cell type or tissue. Targeting of liposomes using a variety of targeting moieties, such as ligands, cell surface receptors, glycoproteins, vitamins, (e.g., ribolflavin) and moncoleonal antibodies, has been previously described (see, e.g., U.S. Pat. Nos. 4,957,773 and 4,603,044). The targeting moeities can comprise the entire protein or fragments thereof. In one aspect, the targeting moiety is a small protein, or peptide. In another aspect, the targeting moiety is a small-molecule.

Cationic lipids are those having one or more moieties that are positively charged at a physiologically relevant pH, typically between 4-8. Particular cationic lipids are as shown in Formula I. Examples of cationic lipids that are useful for the practice of this invention include, but are not limited to, N,N-dioleyl-N,N-dimethylammonium chloride, DC-Chol, 1,3-dioleoyloxy-2-(6-carboxyspermyl)-propyl amide, dioctadecylamidoglycyl spermine, N,N-distearyl-N,N-dimethylammonium bromide, N-(2,3-dioleyloxy)propyl)-N,N-dimethylammonium chloride, 1,2-dioleoyl-3-trimethylammonium-propane chloride, 1,2-dilineoyl-3-dimethylammonium-propane, N-(1-(2,3-dioleyloxy)propyl)-N,N,N-trimethylammonium chloride, 1,2-dioleoyl-3-dimethylammonium propane, 1,2-distearyloxy-N,N-dimethyl-3-aminopropane; didodecyldimethylammonium bromide, dioleoyloxy-N-(2-sperminecarboxamido)ethyl)-N,N-dimethyl-1-propanaminiumtrifluoroacetate, 1,2-dimyristyloxypropyl-3-dimethyl-hydroxyethyl ammonium bromide, 1,2-dioleoylcarbamyl-3-dimethylammoniumpropane, tetramethyltetrapalmitoyl spermine, tetramethyltetraoleyl spermine, tetramethyldioleyl spermine, tetramethyltetramyristyl spermine, tetramethyltetralauryl spermine, 1-(2,3-bis((9Z,12Z)-octadeca-9,12-dienyloxy)propyl)pyrrolidine, 1-(2,3-bis((9Z,12Z)-octadeca-9,12-dienyloxy)propyl)-1H-imidazole, 1-(2,3-bis((9Z,12Z)-octadeca-9,12-dienyloxy)propyl)-4-methylpiperazine, 1-(2,3-bis((9Z,12Z)-octadeca-9,12-dienyloxy)propyl)-4-methyl-1,4-diazepane, 1-(2,3-bis((9Z,12Z)-octadeca-9,12-dienyloxy)propyl)-4-phenylpiperazine, 1-(2,3-bis((9Z,12Z)-octadeca-9,12-dienyloxy)propyl)-4-pyridin-2-ylpiperazine, 1-(2,3-bis((9Z,12Z)-octadeca-9,12-dienyloxy)propyl)piperidine, 4-(2,3-bis((9Z,12Z)-octadeca-9,12-dienyloxy)propyl)morpholine, 1-((2R)-2,3-bis((9Z,12Z)-octadeca-9,12-dienyloxy)propyl)pyrrolidine, 1-((2S)-2,3-bis((9Z,12Z)-octadeca-9,12-dienyloxy)propyl)pyrrolidine, 1-(2,3-bis((9Z,12Z)-octadeca-9,12-dienyloxy)propyl)-4-ethylpiperazine, N-(2,3-bis((9Z,12Z)-octadeca-9,12-dienyloxy)propyl)-N-methyl-N-(3-(pyrrolidin-1-ylmethyl)benzyl)amine, N-(2-(4-(2,3-bis((9Z,12Z)-octadeca-9,12-dienyloxy)propyl)piperazin-1-yl)ethyl)-N,N-dimethylamine, 1-((2S)-2,3-bis((9Z,12Z)-octadeca-9,12-dienyloxy)propyl)-4-methylpiperazine, 1-((2R)-2,3-bis((9Z,12Z)-octadeca-9,12-dienyloxy)propyl)-4-methylpiperazine, 1-(2,3-bis((9Z,12Z)-octadeca-9,12-dienyloxy)propyl)-4-(2-pyrrolidin-1-ylethyl)piperazine, 2-(4-(2,3-bis((9Z,12Z)-octadeca-9,12-dienyloxy)propyl)piperazin-1-yl)pyrimidine, 1-(2,3-bis((9Z,12Z)-octadeca-9,12-dienyloxy)propyl)-N,N-diethylpyrrolidin-3-amine, 1-((9Z,12Z)-octadeca-9,12-dienyloxy)-3-pyrrolidin-1-ylpropan-2-ol, 2-[(9Z,12Z)-octadeca-9,12-dienoyloxy]-1-(pyrrolidin-1-ylmethyl)ethyl (9Z,12Z)-octadeca-9,12-dienoate, 2-[(9Z,12Z)-octadeca-9,12-dienyloxy]-1-(pyrrolidin-1-ylmethyl)ethyl (9Z,12Z)-octadeca-9,12-dienoate, 1-({2-[(8Z,11Z)-heptadeca-8,11-dienyl]-2-[(9Z,12Z)-octadeca-9,12-dienyl]-1,3-dioxolan-4-yl}methyl)pyrrolidine, 1-{2,3-bis[(5Z,8Z,11Z,14Z)-icosa-5,8,11,14-tetraenyloxy]propyl}pyrrolidine, 1-{3-[(5Z,8Z,11Z,14Z)-icosa-5,8,11,14-tetraenyloxy]-2-[(9Z,12Z)-octadeca-9,12-dienyloxy]propyl}pyrrolidine, 1-{2,3-bis[(9E,12E)-octadeca-9,12-dienyloxy]propyl}pyrrolidine, 1-{2-[(9E,12E)-octadeca-9,12-dienyloxy]-3-[(9Z,12Z)-octadeca-9,12-dienyloxy]propyl}pyrrolidine, 1-[2,3-bis(tetradecyloxy)propyl]pyrrolidine, 1-[2,3-bis(octadecyloxy)propyl]pyrrolidine, 1-{2,3-bis[(9Z)-octadec-9-enyloxy]propyl}pyrrolidine, 1-[2,3-bis(dodecyloxy)propyl]pyrrolidine, 1-{2,3-bis[(9Z,12Z)-octadeca-9,12-dienyloxy]propyl}pyrrolidin-3-ol, 1-{3-[(9Z,12Z)-hexadeca-9,12-dienyloxy]-2-[(9Z)-octadec-9-enyloxy]propyl}pyrrolidine, 1-{2,3-bis[(9Z,12Z)-octadeca-9,12-dienyloxy]propyl}-N,N-dimethylpyrrolidin-3-amine, and 1-[3-[(9Z,12Z)-hexadeca-9,12-dienyloxy]-2-(tetradecyloxy)propyl]pyrrolidine, and mixtures thereof.

Lipid-Based Particles are a mixture of one or more cationic lipids of Formula (I), one or more non-cationic lipids, one or more PEG-lipid conjugates, and one or more therapeutic agents. Specific Lipid-Based Particles comprise the following lipid mixtures: cationic lipid(s) (about 2-60% by weight), non-cationic lipid(s) (about 5-90% by weight), and PEG-lipid conjugate(s) (about 0.1-20%).

### Tables 1 and 2

### Representative In-vitro Formulation of Lipid-Based Particles

TABLE 1

| Therapeutic | | |
|---|---|---|
| Agent | Mass (mg) | Vol (μL in water 10 mg/mL) |
| siSTABLE | 0.20 | 20 |
| | Mass (mg) | Vol (μL in ethanol 10 mg/mL) |
| Total Lipids | 5.0 | 500 |

TABLE 2

| | Wt % | Mass (mg) | Vol (μL in ethanol, 10 mg/mL) |
|---|---|---|---|
| Non-cationic | 10 | 0.5 | |
| lipid | | | |
| PEG-lipid | 15 | 0.75 | |
| Cholesterol | 30 | 1.5 | |
| | | Total Volume | 275 |
| Cationic lipid | 45 | 2.25 | 225 |

### Preparation of Lipid Mixture Solution

The mixing solution of cationic lipids, cholesterol, non-cationic lipids and PEG-lipids was prepared in ethanol (total concentration at 10 mg/mL). siSTABLE (purchased from ThermoFisher) (sense-5′ GGG GAA AGC UGG CAA GAU UUU-3′ SEQ ID NO. 1, antisense-5′-AAU CUU GCC AGC UUU CCC CUU-3′ SEQ ID NO: 2) % stock solution was prepared in 10 mg/mL of solution by dissolving 10 mg siRNA in 1 mL of RNAse-free UltraPure Water. The calculated amount of siRNA solution was added to 1 mL of citrate buffer (pH 4.0, 20 mM), to provide an siRNA concentration of 0.2 mg/mL, and warmed to 60 C. The calculated amount of lipid solution was warmed to 60 C, transferred to a 0.5 mL syringe with 28½ gauge needle, and injected into the citrate buffer with stirring at 60 C. After 3 minutes, 3 mL of PBS solution at room temperature (pH 7.4) was added into the lipid mixture with stirring. The Lipid-Based Particle solution was cooled to room temperature.

### Analysis of Lipid-Based Particles

The siRNA concentrations were measured using Quanti-iT RiboGreen RNA reagent (Molecular Probes, (R11490)). Vesicle sizes were characterized by dynamic light scattering with a DynaPro™ Plate Reader (Wyatt Technology) in 96-well half-area UV plate (Coring) after diluting the formulation sample (20 μL) in phosphate buffered saline (80 μL) at a pH of about 7-8. A 1% agarose gel-based assay was used for analyzing nuclease degradation and protection. Encapsulation efficiency (EE) was calculated using data obtained from a RiboGreen assay.

### Ribogreen Assay for Measuring SiRNA Concentration and Encapsulation Efficiency of Lipid-Based Particles

RNA concentration and encapsulation efficiency were determined using a Quant-iT® Ribogreen RNA reagent and kit available from Invitrogen. The siRNA was released from the Lipid-Based Particle using one of the following reagents: ethanol, Triton X-100, or phenol/chloroform. The siRNA concentration is quantified using fluorescent reading at 480 nm/520 nm.

### Particle Sizing Assay

Particle sizes and size distributions (PDI) were characterized by using dynamic light scattering (DLS). A DLS plate reader (Dynapro™, Wyatt Technology) was used for the DLS measurement. This DLS plate reader uses an 830 nm laser and the scattering angle is 158°. It also can control temperature from 4° C. to 70° C. A 96-well format was employed for the samples.

Samples for DLS analysis were prepared by mixing 20 μL of each sample stock solution with 80 μL PBS directly in the 96-well plate (#3697, Corning). Sample mixing was accomplished using a microplate shaker (Orbis, Mikura Ltd.). Plates were read at 20° C. with an acquisition time of 50 seconds for each sample, and data was analyzed with Wyatt Technology's Dynamics V6 software. To rule out potential multiple scattering artifacts, a second plate at 4-fold reduced sample concentrations was independently prepared by mixing 5 μL stock solutions with 95 μL PBS. Under our experimental conditions the results at the two concentrations were very similar, and the final reported result for each sample represents the average of values obtained from the two plates.

TABLE 3
In vitro Formulation Data Table of Particle Size,
Encapsulation Efficiency, and Size Distributions

| | | % | |
|---|---|---|---|
| | | Encapsulation | Size (PDI) |
| Example | Formulation | Efficiency | (nm) |
| 2 | a | 90.7 | 177 |
| 3 | b | 97.0 | 115.6 |
| 4 | a | nd | 146 (0.176) |
| 5 | a | nd | 140 (0.135) |
| 6 | a | 99.8 | 146 |
| 8 | a | 16.7 | 167 (0.29) |
| 9 | b | 96.6 | nd |
| 10 | b | 97.5 | 141.4 |
| 11 | a | 97.5 | 135.4 |
| 12 | nd | nd | nd |
| 13 | b | 86.1 | 115.6 |
| 14 | b | 97.8 | 132.6 |
| 15 | b | 94.5 | 116.8 |
| 16 | b | 79.8 | 115.4 |
| 17 | b | 99.2 | 118 |
| 18 | b | 96.6 | 119 |
| 19 | b | 93.7 | 125.6 |
| 20 | b | 96.3 | nd |
| 21 | b | 91.7 | nd |
| 22 | b | 92.3 | nd |
| 23 | b | 88.0 | nd |
| 24 | b | 46.5 | nd |
| 25 | b | 54.5 | nd |
| 26 | nd | nd | nd |
| 27 | b | 99.0 | nd |
| 28 | b | 98.0 | nd |
| 29 | b | 99.0 | nd |
| 30 | b | 99.0 | nd |
| 31 | nd | nd | nd |
| 32 | b | 99.0 | nd |
| 33 | b | 99.0 | nd |
| 34 | nd | nd | nd |

nd = not determined

TABLE 4
In vitro and In vivo Formulations

| Formulation | | Lipid Ratio | Lipid:siRNA |
|---|---|---|---|
| Designation | Composition | (wt %) | ratio |
| A/a | Cationic lipid/Example 56/DSPC/Chol | 45/10/15/30 | 25:1 |
| B/b | Cationic lipid/PEG- | 45/10/15/30 | 25:1 |
| | cholesterol/DSPC/Chol | | |
| C | Cationic lipid/Pal-PEG-Cera/ | 45/10/15/30 | 25:1 |
| | DSPC/Chol | | |
| D | Cationic lipid/Example 49/DSPC/Chol | 45/10/15/30 | 25:1 |
| E | Cationic lipid/Example 36/DSPC/Chol | 45/10/15/30 | 25:1 |
| F | Cationic lipid/Example 37/DSPC/Chol | 45/10/15/30 | 25:1 |
| G | Cationic lipid/Example 38/DSPC/Chol | 45/10/15/30 | 25:1 |
| H | Cationic lipid/PEG-DMPE/DSPC/Chol | 45/10/15/30 | 25:1 |
| I | Cationic lipid/PEG-DPPE/DSPC/Chol | 45/10/15/30 | 25:1 |
| J | Cationic lipid/PEG-DSPE/DSPC/Chol | 45/10/15/30 | 25:1 |
| K | Cationic lipid/PEG-DMG/DSPC/Chol | 45/10/15/30 | 25:1 |
| L | Cationic lipid/PEG-DPG/DSPC/Chol | 45/10/15/30 | 25:1 |
| M | Cationic lipid/PEG-DSG/DSPC/Chol | 45/10/15/30 | 25:1 |
| N | Cationic lipid/PEG-DPG/DOPE/Chol | 45/10/15/30 | 25:1 |
| MM | Cationic lipid/PEG-DPG/DOPC/Chol | 45/10/15/30 | 25:1 |
| O | Cationic lipid/PEG-DPG/SPC/Chol | 45/10/15/30 | 25:1 |
| P | Cationic lipid/PEG-DSPE/DOPE/Chol | 45/10/15/30 | 25:1 |
| Q | Cationic lipid/PEG-DSPE/DOPC/Chol | 45/10/15/30 | 25:1 |
| R | Cationic lipid/PEG-DSPE/SPC/Chol | 45/10/15/30 | 25:1 |
| S | Cationic lipid/PEG-DMG/PEG-DPG/ | 44/4.5/4.5/14/33 | 25:1 |
| | DSPC/Chol | | |
| T | Cationic lipid/PEG-DMG/PEG-DSG/ | 44/4.5/4.5/14/33 | 25:1 |
| | DSPC/Chol | | |
| U | Cationic lipid/PEG-DPG/PEG-DSG/ | 44/4.5/4.5/14/33 | 25:1 |
| | DSPC/Chol | | |
| V | Cationic lipid/PEG-DMPE/PEG-DPPE/ | 44/4.5/4.5/14/33 | 25:1 |
| | DSPC/Chol | | |
| W | Cationic lipid/PEG-DPPE/PEG-DSPE/ | 44/4.5/4.5/14/33 | 25:1 |
| | DSPC/Chol | | |
| X | Cationic lipid/PEG-DMPE/PEG-DSPE/ | 44/4.5/4.5/14/33 | 25:1 |
| | DSPC/Chol | | |
| Y | Cationic lipid/Example 37/PEG-DSG/ | 44/4.5/4.5/14/33 | 25:1 |
| | DSPC/Chol | | |
| Z | Cationic lipid/PEG-DMG/PEG-DSPE/ | 44/4.5/4.5/14/33 | 25:1 |
| | DSPC/Chol | | |
| AA | Cationic lipid/PEG-DSG/PEG-DMPE/ | 44/4.5/4.5/14/33 | 25:1 |
| | DSPC/Chol | | |
| BB | Cationic lipid/Example 37/PEG-DSPE/ | 44/4.5/4.5/14/33 | 25:1 |
| | DSPC/Chol | | |
| CC | Cationic lipid/Example 37/DSPC/Chol | 46/5/15/34 | 25:1 |
| DD | Cationic lipid/Example 37/DSPC/Chol | 44/9/14/33 | 25:1 |
| EE | Cationic lipid/Example 37/DSPC/Chol | 44/13/13/32 | 25:1 |
| FF | Cationic lipid/PEG-DSPE/DSPC/Chol | 46/5/15/34 | 25:1 |
| GG | Cationic lipid/PEG-DSPE/DSPC/Chol | 45/7/14/34 | 25:1 |
| HH | Cationic lipid/PEG-DPPE/PEG- | 44/4.5/4.5/14/33 | 15:1 |
| | DSPE/DSPC/Chol | | |
| II | Cationic lipid/PEG-DPPE/PEG- | 44/4.5/4.5/14/33 | 10:1 |
| | DSPE/DSPC/Chol | | |
| JJ | Cationic lipid/Example 37/DSPC/chol | 44/9/33/14 | 15:1 |
| KK | Cationic lipid/Example 37/DSPC/chol | 44/9/33/14 | 10:1 |
| LL | Cationic lipid/PEG-DMPE/PEG- | 44/4.5/4.5/14/33 | 25:1 |
| | DPG/DSPC/chol | | |

TABLE 5
Representative In vivo Formulation of Lipid-Based Particles

| | Cationic | | Non-cationic | | |
|---|---|---|---|---|---|
| | lipid | PEG-lipid | lipid | Cholesterol | |
| % (w/w) | 44 | 9 | 14 | 33 | |
| Weight (mg) | 54.9 | 11.2 | 17.6 | 41.4 | Total |
| Volume (ml) | 5.49 | 1.12 | 1.76 | 4.14 | 12.5 ml |

### Preparation of Lipid Mixture Solution

The lipid solution was prepared (10 mg/ml) by dissolving the lipid in 200 proof ethanol. The lipid mixture solution is prepared according to the above composition in Table 5.

### Preparation of siRNA Solution

An siRNA (TetR_ODC_{—}12, G.G.G.G.A.A.A.G.C.U.G.G.C.A.A.G.A.U.U.U.U) (ThermoFisher) solution is prepared in a concentration of 10 mg/ml by dissolving 10 mg siRNA in 1 ml of DNAse/RNAse-free distilled water.

### Preparation of Lipid-Based Particles

A round bottom flask was submerged into a 65° C. water bath. Citrate buffer (37.5 ml) of pH 4.0 was pipetted into the flask. The solution was stirred by a magnetic stirring bar at a speed of 900 rpm. Both the pH 4.0 citrate buffer and the lipid solution were pre-warmed in the 65° C. water bath for about 3 minutes. A siRNA solution (0.5 ml) was pipetted into the pH 4.0 citrate buffer. The 12.5 ml lipid mixture solution was injected through a 27 gauge needle into the citrate buffer in about 30 seconds. The needle tip was inserted into the solution during the injection. The resulting solution was stirred for 5 minutes at a speed of 900 rpm. The flask was pulled up from the water bath and a 50 ml pH 7.4 PBS buffer was added into the flask. The final solution was further mixed at a speed of 900 rpm for 5 minutes. For the diafiltration process, a dialysis filter (Millipore, 100K, Cat# PXB100C50) was used to remove ethanol in the above solution. When the volume was reduced to 20 ml during the initial diafiltration, 20 ml of pH 7.4 PBS was added to the sample solution. The diafiltration was continued until the volume was reduced to 20 ml. The diafiltration process was repeated 4 times. The volume of the sample solution was reduced to about 12 ml and pH 7.4 PBS was added to make the final volume of 15 ml. The 15 ml solution was filtered sequentially through the 0.45 and 0.22 μm sterile PVDF membrane filters (Millipore) and immediately transferred into a sterile vial.

### Particle Sizing Assay

For measurements of particle sizes and size distributions (PDI), lipid-based particles were prepared as described above. The particle solution (60 μL) was pipetted into a disposable cuvette (UVette, Eppendorf, cat# 952010051) and measured in the “General Purpose” mode. Attenuator and position were optimized by the device. Measurements were performed using a Zetasizer Nano ZS (Malvern Instruments) equipped with a 4 mW He-Ne laser at a wavelength of 633 nm at 25° C. Scattered light was detected at a 173° backward scattering angle. The viscosity and refractive index of water at 25° C. was used for data analysis with the Dispersion Technology Software 5.00 (Malvern Instruments).

TABLE 6
In vivo Formulation Data Table of Particle Size,
Encapsulation Efficiency, and Size Distributions

| | | % Encapsulation | Size (PDI) | |
|---|---|---|---|---|
| Example | Formulation | Efficiency | (nm) | |
| 1 | A | 91.7 | 102 | (0.15) |
| 1 | B | 91.3 | 112 | (0.041) |
| 1 | C | 85.1 | 166 | (0.077) |
| 1 | E | 91.7 | 116 | (0.107) |
| 1 | F | 90.2 | 109 | (0.028) |
| 1 | G | 91.5 | 98 | (0.036) |
| 1 | H | 87.8 | 138 | (0.129) |
| 1 | I | 86.7 | 108 | (0.053) |
| 1 | J | 89.8 | 110 | (0.024) |
| 1 | K | 91.0 | 120 | (0.065) |
| 1 | L | 90.9 | 108 | (0.092) |
| 1 | M | 89.7 | 136 | (0.119) |
| 1 | N | 100 | 124.8 | (0.035) |
| 1 | O | 100 | 117.6 | (0.079) |
| 1 | S | 163.4 | 96.8 | (0.036) |
| 1 | U | 276.4 | 99.9 | (0.016) |
| 1 | V | 239.4 | 115.1 | (0.038) |
| 1 | W | 268.2 | 107.1 | (0.041) |
| 1 | X | 217.7 | 104.5 | (0.01) |
| 1 | Y | 263.6 | 119.7 | (0.068) |
| 1 | Z | 97 | 106.7 | (0.032) |
| 1 | AA | 98 | 109 | (0.047) |
| 1 | BB | 94 | 120.5 | (0.043) |
| 1 | CC | 89 | 173.3 | (0.009) |
| 1 | DD | 97 | 102.1 | (0.034) |
| 1 | EE | 99 | 86.04 | (0.085) |
| 1 | FF | 99 | 242.8 | (0.21) |
| 1 | GG | 97 | 149.2 | (0.018) |
| 1 | HH | 98 | 95.5 | (0.038) |
| 1 | II | 98 | 83 | (0.019) |
| 1 | JJ | 98 | 88.4 | (0.02) |
| 1 | KK | 97 | 84.1 | (0.027) |
| 1 | LL | 95 | 191.7 | (0.30) |
| 1 | MM | 100 | 130.4 | (0.045) |
| 3 | A | 79.3 | 157 | (0.15) |
| 9 | A | nd | 95 | (0.07) |
| 10 | A | nd | 97 | (0.05) |
| 19 | A | 92.7 | 133 | (0.167) |
| 27 | I | 99.0 | 105.7 | (0.02) |
| 27 | J | 99.0 | 90.2 | (0.054) |
| 27 | L | 99.0 | 100.7 | (0.036) |
| 32 | I | 98.0 | 127.6 | (0.036) |
| 32 | J | 98.0 | 119 | (0.047) |
| 32 | L | 99.0 | 118.6 | (0.048) |
| 57 | A | 92.5 | 107 | (0.07) |

nd = not determined

### Determination of Transfection Efficiency of MDA435-TetR-Luc cells with Lipid-Based Particles

To determine the knockdown efficacy of Lipid-Based Particles in an in vitro assay, MDA435-TetR-Luc cells (The positive readout reporter cell line MDA435-TetR-Luc contained a stably integrated copy of the luciferase gene expressed from a CMV promoter containing the tetR operator site. In addition, gene coding for a destabilized TetR protein was expressed in this cell line.) were plated in 96 well plate at a density of 10K cells per well in 100 ul of DMEM (Dulbecco's Modified Eagles Medium, Invitrogen Corp.) containing 10% fetal bovine serum (Invitrogen Corp.). Appropriate dilutions of Lipid-Based Particles were made in DMEM+10% fetal bovine serum medium, 10 ul of the diluted material was transferred into each well in triplicate. Transfected cells were further incubated at 37° C. for a period of 72 hours. Supernatent from each well was removed and cells were assayed for luciferase activity (Steady Glo kit, ProMega Corp.) as per the manufacturers recommendation. Positive controls included cells treated with 100 ul of doxycycline at 0.5 mg/ml, 20 nM tetR siRNA transfected with lipofectamine (Invitrogen Corp.) or untreated cells. The graphs represent average of triplicate readings of the Lipid-Based Particles treated sample divided by the average of readings from 9 wells treated with doxycycline.

### Tumor Models

The animal studies were carried out in accordance with internal Institutional Animal Care and Use Committee (IACUC) guidelines at Abbott Laboratories. Scid female mice at 6 to 8 weeks of age were obtained from Charles River Laboratory and used for intraliver tumor models. Mouse livers were exposed by vertical incision on mouse abdomens and the tumor cells were directly injected into the livers. The incision was closed by suture and wound clips. All cell lines used for creating xenograft tumors were subjected to the IMPACT profile I test (18 agents) at the University of Missouri Research Animal Diagnostic and Investigative Laboratory, and all cell lines were found negative for the 18 infectious agents tested. Tumor cells were suspended in a 1:1 mixture of S-MEM (Invitrogen, Carlsbad, Calif.) and matrigel (BD Bioscience, San Jose, Calif.) and inoculated at 1×10E6 cells per animal.

### Animal Dosing and Sample Harvesting

Treatments were started 3˜4 weeks after tumor inoculation. Formulated or unformulated siRNAs were administrated via tail vein (i.v) injection.

### IHC Analysis

IHC was carried out as previously described [Li, L., et al., Evaluating hypoxia-inducible factor-1 alpha as a cancer therapeutic target via inducible RNA interference in vivo. Cancer Res, 2005. 65 (16): p. 7249-58]. Briefly, tumors were excised, cut into pieces of less than 3 mm in thickness and immediately fixed in buffered formalin solution with neutral pH (Sigma, St. Louis, Mo.). The formalin-fixed and paraffin-embedded tumor sections were then used for staining. The mouse anti-β-galactosidase mAb (Promega, Madison, Wis.) was used to detect β-galactosidase in tumor sections. DAB (3,3′-diaminobenzidine) was used as the chromogen. IHC images were acquired using the Nikon TE2000 inverted microscope. The β-galactosidase staining was evaluated by 2 people independently based on the scoring system listed below. The average of the score was calculated for each tumor.

### IHC Scoring System

−, there was no staining, a small area of weak staining, or disperse strong single cell staining
+/−, there was 5% of the section with weak staining or one patch of strong staining
+, there was 15% of the section with strong staining
++, there was 50% of the section with strong straining
+++, there was 80% of the section with strong straining
++++, the whole section had strong staining

TABLE 7
In vivo Response of a Lipid-Based Particles (1-A,
3-A, 7-A) versus a Positive Control (Doxycycline).

| | 1 | 2 | 3 | 4 | 5 | 6 |
|---|---|---|---|---|---|---|
| Doxycycline | +++ | +++ | +++ | +++ | +++ | +++ |
| 1-A | + | + | + | + | + | −/+ |
| 3-A | +/− | +/− | | − | − | − |
| 7-A | + | −/+ | −/+ | −/+ | − | − |
| 57-A | + | + | −/+ | −/+ | − | − |

### Bioluminescence Imaging and Analysis

In vivo bioluminescence imaging and analysis were conducted on the IVIS 200 system using the Living Image acquisition and analysis software (Caliper Life Science, Hopkinton, Mass.). After intra-peritoneal injection of luciferin (Promege, Madison, Wis.) at 150 mg/kg, mice were anesthetized with isofluorane. Four minutes after the injection of luciferin, a series of time-lapse images were acquired at 2 minutes intervals in a total of 10 minutes. Regions of interest (ROI) were drawn around the tumors and signal intensity was quantified as the sum of photon counts per second within the ROI after the subtraction of background luminescence. The peak reading during the 10-minute imaging period was used for calculating the signal ratio before and after siRNA delivery.

The ability of novel cationic lipids to transfect siRNA in vitro was evaluated in the TetRLuc assay. By utilizing a releasable PEG lipid as for examples in formulations “a” or “b”, the transfection efficiency of the unshielded or partially shielded particle may be determined.

The transfection ability of cationic lipids is not readily predicted based what is currently known in the art. Take for example cationic lipid transfection agents that typically contain either a dimethylamino- or trimethylammonium cationic head group (e.g., DOTAP, DODAP, and DOTMA (Avanti Polar Lipids Inc., Alabaster, Ala.). However, changing the head group to the related diethylamino cationic head group may diminish activity (vide infra, Example 57). A formulation that employs a cationic lipid having a heterocyclic amine, such as pyrrolidine (Example 1) unexpectedly provided in vitro transfection activity as well as efficient in vivo delivery.

Without intending on being held to any particular theory, the in vitro transfection efficiency of a given formulation, including the cationic lipids of the present invention, may or may not predict for in vivo delivery. The in vivo delivery may depend upon the properties of other co-lipid components in the formulation. Properties of the co-lipids that may modulate in vivo delivery, include for example, PEG lipid alkyl length, PEG polymer length, concentration of the PEG lipid conjugate, presence and concentration of neutral helper lipid, as well as the manner of which the co-lipid components are formulated (Sadzuka, et. al., J. Liposome Research, 13,2, (2003) 157-172; Sadzuka, et. al., Int. J. Pharm., 312, (2006) 83-89; Li, et. al, Biochimica et Biophysica Acta 1513 (2001) 193-206; Chiu, et al., Biochimica et Biophysica Acta 1560 (2002) 37-50; and Mukherjee, et al., FEBS Letters 579 (2005) 1291-1300.)

The aggregate effect of these co-lipids and their formulation impacts a set of parameters that includes for example particle stabilization, serum stability, circulation half-life, particle internalization, intracellular release of the therapeutic agent. These factors in total are likely to mitigate effective in vivo delivery.

### Synthesis

The following abbreviations have the meanings indicated: ADDP means 1,1′-(azodicarbonyl)dipiperidine; AD-mix-β means a mixture of (DHQD)₂PHAL, K₃Fe(CN)₆, K₂CO₃ and K₂SO₄); AIBN means 2,2′-azobis(2-methylpropionitrile); 9-BBN means 9-borabicyclo(3.3.1)nonane; Cp means cyclopentadiene; (DHQD)₂PHAL means hydroquinidine 1,4-phthalazinediyl diethyl ether; DBU means 1,8-diazabicyclo(5.4.0)undec-7-ene; DCC means dicyclohexylcarbodiimide; DIBAL means diisobutylaluminum hydride; DIEA means diisopropylethylamine; DMAP means N,N-dimethylaminopyridine; DME means 1,2-dimethoxyethane; DMF means N,N-dimethylformamide; dmpe means 1,2-bis(dimethylphosphino)ethane; DMSO means dimethylsulfoxide; dppa means diphenylphosphoryl azide; dppb means 1,4-bis(diphenylphosphino)butane; dppe means 1,2-bis(diphenylphosphino)ethane; dppf means 1,1′-bis(diphenylphosphino)ferrocene; dppm means 1,1-bis(diphenylphosphino)methane; EDAC means 1-(3-dimethylaminopropyl)-3-ethylcarbodiimide; Fmoc means fluorenylmethoxycarbonyl; HATU means O-(7-azabenzotriazol-1-yl)-N,N′N′N′-tetramethyluronium hexafluorophosphate; HMPA means hexamethylphosphoramide; IPA means isopropyl alcohol; LDA means lithium diisopropylamide; LHMDS means lithium bis(hexamethyldisilylamide); MP-BH₃ means macroporus triethylammonium methylpolystyrene cyanoborohydride; LAH means lithium aluminum hydride; NCS means N-chlorosuccinimide; PyBOP means benzotriazol-1-yloxytripyrrolidinophosphonium hexafluorophosphate; TDA-1 means tris(2-(2-methoxyethoxy)ethyl)amine; TEA means triethylamine; TFA means trifluoroacetic acid; THF means tetrahydrofuran; NCS means N-chlorosuccinimide; NMM means N-methylmorpholine; NMP means N-methylpyrrolidine; PPh₃ means triphenylphosphine.

The following schemes and examples are presented to provide what is believed to be the most useful and readily understood description of procedures and conceptual aspects of this invention.

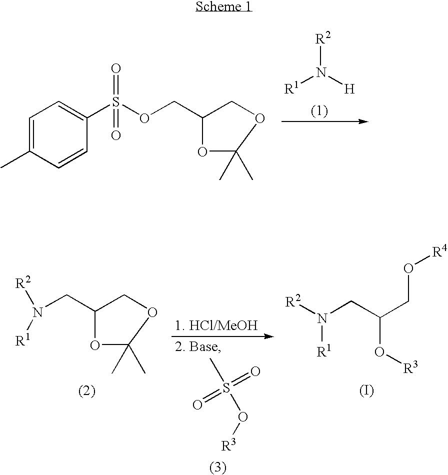

As shown in Scheme 1, (2,2-dimethyl-1,3-dioxolan-4-yl)methyl 4-methylbenzenesulfonate, when reacted with an amine of Formula (1) wherein R¹ and R² are as described herein, will provide a compound of Formula (2). The reaction may be conducted in a commercial single mode microwave at elevated temperature. Anhydrous solvents such as but not limited to dioxane are typically employed.

Compounds of Formula (I), wherein R¹, R², R³, and R⁴ are as described herein, can be prepared by reacting compounds of Formula (2), after first treating with methanolic acid and then with base such as but not limited to sodium hydride, with a compound of Formula (3). The reaction is typically performed in at an elevated temperature in a solvent such as but not limited to toluene.

If it is desired for R³ and R⁴ to be the same, two equivalents of (3) can be used. If R³ and R⁴ are to be different, one equivalent of (3) can be used to obtain a compound wherein R⁴ is H after purification. This intermediate can then be reacted with CH₃(SO₃)R⁴ to obtain a compound of Formula (I).

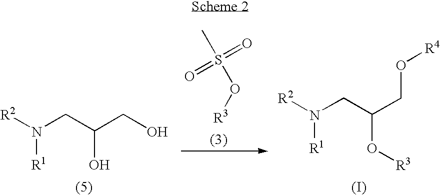

Alternatively, the diol of Formula (5) may commercially available or synthesized as described in Example 9 and reacted with a compound of Formula (3) to obtain a compound of Formula (1). The reaction typically requires the use of a base such as but not limited to sodium hydride and a solvent such as but not limited to toluene at elevated temperatures.

If it is desired for R³ and R⁴ to be the same, two equivalents of (3) can be used. If R³ and R⁴ are to be different, one equivalent of (3) can be used to obtain a compound wherein R⁴ is H after purification. This intermediate can then be reacted with CH₃(SO₃)R⁴ to obtain a compound of Formula (I).

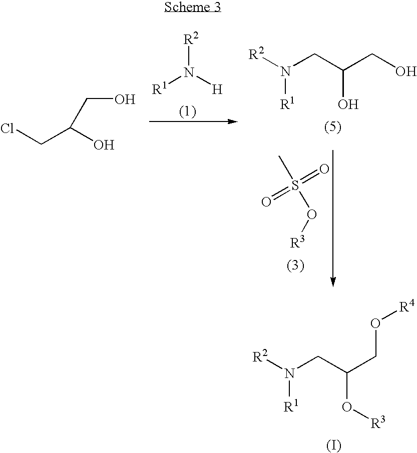

As shown in Scheme 3-chloropropane-1,2-diol can be reacted with a compound of Formula (1), wherein R¹ and R² when taken together are heteroaryl or heterocycloalkyl, and a base such as but not limited to sodium hydride, to provide a compound of Formula (5).

Compounds of Formula (I), wherein R¹ and R² when taken together are heteroaryl or heterocycloalkyl, can be prepared by reacting compounds of Formula (5) and base, such as but not limited to sodium hydride, with a compound of Formula (3). The reaction is typically performed in at an elevated temperature in a solvent such as but not limited to toluene.

If it is desired for R³ and R⁴ to be the same, two equivalents of (3) can be used. If R³ and R⁴ are to be different, one equivalent of (3) can be used to obtain a compound wherein R⁴ is H after purification. This intermediate can then be reacted with CH₃(SO₃)R⁴ to obtain a compound of Formula (I).

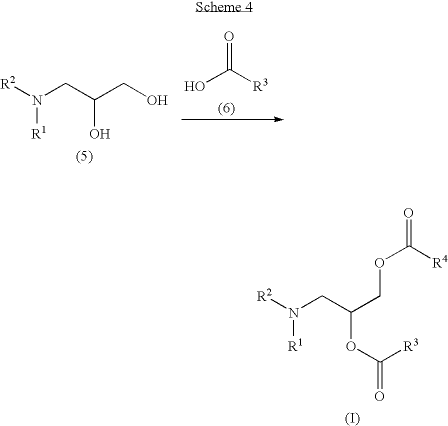

Alternatively, the diol of Formula (5), which is commercially available or synthesized as described in Example 9, can be reacted with a compound of Formula (6) to obtain a compound of Formula (I), as shown in Scheme 4. The reaction typically requires the use solvent such as but not limited to dichloromethane in the presence of DMAP and EDC.

If it is desired for R³ and R⁴ to be the same, two equivalents of (6) can be used. If R³ and R⁴ are to be different, one equivalent of (6) can be used to obtain a compound wherein R⁴ is H after purification. This intermediate can then be reacted with R⁴C(O)OH to obtain a compound of Formula (I).

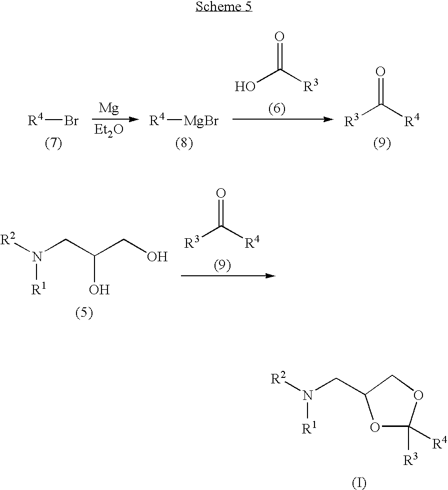

As shown in Scheme 5, a bromide of Formula (7), which may be commercially available, can be reacted with magnesium in diethyl ether to obtain the compound of Formula (8). A compound of Formula (6) can be reacted with oxalyl chloride in CHCl₃ followed by the addition of a compound of Formula (8) to obtain a compound of Formula (9).

The diol of Formula (5), which may be commercially available or synthesized as described in Example 9, can be reacted with a compound of Formula (9), which may be commercially available, to obtain a compound of Formula (I). The reaction typically requires the use solvent such as but not limited to toluene at elevated temperatures in the presence of an acid catalyst.

### Example 1

### 1-(2,3-bis((9Z,12Z)-octadeca-9,12-dienyloxy)propyl)pyrrolidine

3-(Pyrrolidin-1-yl)propane-1,2-diol (150 mg) and linoleyl methane sulfonate (1.068 g) were combined in toluene (5 mL). Sodium hydride (104 mg, 95% w/w) was added, and the mixture was stirred for 5 minutes, heated in a sealed vial at 100° C. for 2 hours, cooled to room temperature, quenched with methanol and partitioned between ethyl acetate (100 mL) and water (50 mL). The extract was dried over Na₂SO₄, filtered and concentrated. The concentrate was purified by flash chromatography on silica gel (0-5% methanol in dichloromethane). MS (ESI) m/e 642 (M+H)⁺; ¹H NMR (300 MHz, CDCl₃) δ 5.14-5.74 (m, 8H) 3.25-3.75 (m, 7H) 2.32-2.94 (m, 10H) 1.88-2.27 (m, 8H) 1.47-1.88 (m, 8H) 1.13-1.47 (m, 32H) 0.77-1.08 (m, 6H).

### Example 21

### (2,3-bis((9Z,12Z)-octadeca-9,12-dienyloxy)propyl)-1H-imidazole

### Example 2A

### 1-((2,2-dimethyl-1,3-dioxolan-4-yl)methyl)-1H-imidazole

A mixture of 1H-imidazole (680 mg), 4-(chloromethyl)-2,2-dimethyl-1,3-dioxolane (1.8 g) and 60% oily sodium hydride (800 mg) in DMF (5 mL) was stirred at room temperature for 10 minutes and at 60° C. for 4 hours, cooled, quenched with methanol and treated with dichloromethane (100 mL) and water (50 mL). The extract was concentrated, and the concentrate was purified by flash chromatography on silica gel (0-25% methanol/dichloromethane). MS (ESI) m/e 183 [M+H]⁺; ¹H NMR (400 MHz, DMSO-d₆) δ 7.60 (s, 1H), 7.15 (s, 1H), 6.88 (s, 1H), 4.23-4.42 (m, 1H), 4.10-4.23 (m, 1H), 3.92-4.09 (m, 2H), 3.61 (dd, J=8.59, 6.14 Hz, 1H), 1.28 (d, J=17.49 Hz, 6H).

### Example 2B

### 1-(2,3-bis((9Z,12Z)-octadeca-9,12-dienyloxy)propyl)-1H-imidazole

A mixture of EXAMPLE 2A (183 mg) and 0.1 M HCl (5 mL) in methanol was stirred at room temperature for 2 hours and concentrated. To the concentrate and linoleyl methane sulfonate (1 g) in toluene (5 mL) was added 60% oily sodium hydride (160 mg). The mixture was stirred at room temperature for 5 minutes, heated in a sealed vial at 100° C. for 2 hours, cooled to room temperature, quenched with methanol then water and extracted with ethyl acetate. The extract was dried over Na₂SO₄, filtered and concentrated. The concentrate was purified by flash chromatography on silica gel (0-15% methanol/dichloromethane). MS (ESI) m/e 639.6 (M+H)⁺; ¹H NMR (300 MHz, CDCl₃) δ 7.49 (s, 1H), 7.04 (s, 1H), 6.95 (s, 1H), 5.17-5.52 (m, 8H), 3.88-4.27 (m, 2H), 3.03-3.73 (m, 6H), 2.67-2.93 (m, J=5.95, 5.95 Hz, 3H), 1.93-2.23 (m, 8H), 1.43-1.71 (m, 6H), 1.15-1.45 (m, 32H), 0.80-1.03 (m, 6H).

### Example 3

### 1-(2,3-bis((9Z,12Z)-octadeca-9,12-dienyloxy)propyl)-4-methylpiperazine

A mixture of 1-methyl-piperazine (700 mg) and (2,2-dimethyl-1,3-dioxolan-4-yl)methyl 4-methylbenzenesulfonate (500 mg) in dioxane (4 mL) was heated in a microwave (Biotage Initiator) at 140° C. for 40 minutes and concentrated. The concentrate was dissolved in 0.1 N HCl in methanol, stirred overnight at room temperature and concentrated. The concentrate was added to saturated sodium bicarbonate solution and extracted with chloroform. The extract was dried over Na₂SO₄, filtered and concentrated. A mixture of the concentrate and linoleyl methane sulfonate (800 mg) in toluene (in 5 mL) was treated with 60% oily sodium hydride (120 mg), stirred for 5 minutes at room temperature and at 100° C. in a sealed vial for 2 hours, cooled to room temperature, quenched with methanol, treated with water and extracted with ethyl acetate. The extract was dried over Na₂SO₄, filtered and concentrated. The concentrate was purified by flash chromatography on silica gel (0-15% methanol/dichloromethane. MS (ESI) m/e 671.6 (M+H)⁺; ¹H NMR (300 MHz, CDCl₃) δ 5.22-5.51 (m, 8H), 3.34-3.70 (m, 6H), 2.70-2.87 (m, J=6.71, 6.71 Hz, 4H), 2.35-2.65 (m, 8H), 2.05 (q, J=7.02 Hz, 8H), 2.05 (q, J=7.02 Hz, 8H), 1.48-1.63 (m, 2H), 1.21-1.44 (m, 32H), 0.89 (t, J=6.87 Hz, 6H).

### Example 4

### 1-(2,3-bis((9Z,12Z)-octadeca-9,12-dienyloxy)propyl)-4-methyl-1,4-diazepane

Prepared as described in EXAMPLE 3 by substituting 1-methyl-1,4-diazepane for 1-methyl-piperazine. MS (ESI−) m/e 707.6 (M+Na)⁺; ¹H NMR (400 MHz, CDCl₃) δ 5.21-5.48 (m, 8H), 3.35-3.66 (m, 6H), 3.08-3.22 (m, J=6.60, 6.60 Hz, 1H), 2.70-2.85 (m, J=6.44, 6.44 Hz, 6H), 2.26-2.67 (m, 8H), 2.21 (s, 3H), 2.05 (q, J=6.96 Hz, 8H), 1.49-1.63 (m, 6H), 1.20-1.41 (m, 32H), 0.89 (t, 6H).

### Example 5

### 1-(2,3-bis((9Z,12Z)-octadeca-9,12-dienyloxy)propyl)-4-phenylpiperazine

Linoleyl methane sulfonate (1 g) was added to a mixture of 3-(4-phenylpiperazin-1-yl)propane-1,2-diol (241 mg) and 60% oily sodium hydride (200 mg) in toluene (5 mL). After 5 minutes, the mixture was heated at 100° C. in a sealed vial for 2 hours, cooled to room temperature, quenched with methanol and treated with ethyl acetate and water. The extract was washed with brine and dried over Na₂SO₄, filtered and concentrated. The concentrate was purified by flash chromatography on silica gel (0-15% methanol in dichloromethane). MS (ESI−) m/e 734.6 (M+H)⁺; ¹H NMR (400 MHz, CDCl₃) δ 7.16-7.34 (m, 2H), 6.75-7.02 (m, 3H), 5.20-5.50 (m, 8H), 3.31-3.77 (m, 10H), 3.18 (t, J=4.91 Hz, 3H), 2.42-2.86 (m, 8H), 2.05 (q, J=6.85 Hz, 8H), 1.47-1.65 (m, 4H), 1.12-1.45 (m, 32H), 0.69-1.03 (m, 6H).

### Example 6

### 1-(2,3-bis((9Z,12Z)-octadeca-9,12-dienyloxy)propyl)-4-pyridin-2-ylpiperazine

Prepared as described in EXAMPLE 3 by substituting 1-(pyridin-2-yl)piperazine (1.1 g) for 1-methyl-piperazine. MS (ESI−) m/e 734.6 (M+H)⁺; ¹H NMR (400 MHz, CDCl₃) δ 8.18 (dd, J=4.45, 1.69 Hz, 1H), 7.35-7.54 (m, 1H), 6.48-6.75 (m, 2H), 5.13-5.57 (m, 8H), 3.26-3.79 (m, 11H), 2.35-2.88 (m, 10H), 2.05 (q, J=6.96 Hz, 8H), 1.55 (d, J=5.83 Hz, 4H), 1.17-1.45 (m, 32H), 0.71-1.01 (m, 6H).

### Example 7

### 1-(2,3-bis((9Z,12Z)-octadeca-9,12-dienyloxy)propyl)piperidine

A mixture of 3-(piperidin-1-yl)propane-1,2-diol (0.2 g) and NaH (0.32 g) in toluene (12.56 ml) was stirred at room temperature for 30 minutes, treated with (9Z,12Z)-octadeca-9,12-dienyl methanesulfonate (0.952 g) in toluene (1 mL), heated at 90° C. for 4 hours, cooled to room temperature, quenched with methanol and treated with ethyl acetate and water. The extract was dried over MgSO₄, filtered and concentrated onto silica. The concentrate was purified on Analogix with 1000%-50% hexane/ethyl acetate (SF25-34 g column). MS (ESI(+))m/e 656 (M+H)⁺; ¹H NMR (300 MHz, CDCl₃) δ 5.25-5.45 (m, 8H), 3.38-3.64 (m, 8H), 2.72-2.82 (m, 4H), 2.32-2.49 (m, 6H), 1.98-2.12 (m, 8H), 1.48-1.60 (m, 4H), 1.22-1.45 (m, 37H), 0.76-1.05 (m, 6H).

### Example 8

### 4-(2,3-bis((9Z,12Z)-octadeca-9,12-dienyloxy)propyl)morpholine

To a mixture of 3-morpholinopropane-1,2-diol (0.174 g) in toluene (6 ml) at 0° C. was added sodium hydride (0.218 g). The mixture was stirred at room temperature for 1 hour, cooled to 0° C., treated with (9Z,12Z)-octadeca-9,12-dienyl methanesulfonate (0.930 g), heated at reflux for 2 hours, cooled to 0° C., quenched with ethanol, and concentrated. The concentrate was purified by flash chromatography (Analogix SF25-40 g, 0-5% methanol/dichloromethane and Analogix RS12-25 g, 10-20% ethyl acetate/hexanes). MS (ESI(−))m/e 658.6 (M+H)⁺; ¹H NMR (300 MHz, CDCl₃) δ 5.28-5.43 (m, 8H), 3.69 (t, J=4.76 Hz, 4H), 3.41-3.62 (m, 7H), 2.77 (t, J=5.95 Hz, 4H), 2.40-2.55 (m, 6H), 2.05 (q, J=6.74 Hz, 8H), 1.51-1.57 (m, 4H), 1.25-1.38 (m, 32H), 0.86-0.92 (m, 6H).

### Example 9

### 1-((2R)-2,3-bis((9Z,12Z)-octadeca-9,12-dienyloxy)propyl)pyrrolidine

Pyrrolidine (475 mg) and (S)-3-chloropropane-1,2-diol (442 mg) in dioxane (3 mL) in a microwave vial were heated in a microwave (Biotage Initiator) at 140° C. for 40 minutes. The mixture was partitioned between water and chloroform. The extract was dried over Na₂SO₄, filtered and concentrated. A mixture of the concentrate, linoleyl methane sulfonate (1.068 g), 60% oily sodium hydride (200 mg) in toluene (5 mL) was heated at 100° C. for 2 hours, cooled to room temperature, quenched with methanol and treated with ethyl acetate and water. The extract was washed with brine, dried over Na₂SO₄, filtered and concentrated. The concentrate was purified by flash chromatography on silica gel. (0-15% methanol in dichloromethane). MS (ESI(−))m/e 642.6 (M+H)⁺; ¹H NMR (300 MHz, CDCl₃) δ 5.18-5.49 (m, 8H), 3.30-3.79 (m, 7H), 2.77 (t, J=5.98 Hz, 10H), 2.05 (q, J=6.96 Hz, 8H), 1.83 (m, 4H), 1.47-1.67 (m, 4H), 1.18-1.45 (m, 32H), 0.79-0.99 (m, 6H).

### Example 10

### 1-((2S)-2,3-bis((9Z,12Z)-octadeca-9,12-dienyloxy)propyl)pyrrolidine

Prepared as described in EXAMPLE 9 by substituting (R)-3-chloropropane-1,2-diol for (S)-3-chloropropane-1,2-diol. MS (ESI(−))m/e 642.6 (M+H)⁺; ¹H NMR (300 MHz, CDCl₃) δ 5.18-5.49 (m, 8H), 3.30-3.79 (m, 7H), 2.77 (t, J=5.98 Hz, 10H), 2.05 (q, J=6.96 Hz, 8H), 1.83 (m, 4H), 1.47-1.67 (m, 4H), 1.18-1.45 (m, 32H), 0.79-0.99 (m, 6H).

### Example 11

### 1-(2,3-bis((9Z,12Z)-octadeca-9,12-dienyloxy)propyl)-4-ethylpiperazine

A mixture of 1-ethylpiperazine (625 mg) and 3-chloropropane-1,2-diol (528 mg) in dioxane (4 mL) was heated in a microwave (Biotage Initiator) at 140° C. for 40 minutes, cooled, added to saturated sodium bicarbonate solution and washed with chloroform. The extract was dried over anhydrous Na₂SO₄, filtered and concentrated. The concentrate was dissolved in toluene (5 mL) and treated with inoleyl methane sulfonate (1 g) and 60% oily sodium hydride (250 mg). After 5 minutes at room temperature, the mixture was heated at 100° C. in a sealed vial for 2 hours. After cooling to room temperature, the mixture was quenched with methanol and treated with ethyl acetate and water. The extract was washed with brine and dried over Na₂SO₄, filtered and concentrated. The concentrate was purified by flash chromatography on silica gel (0-15% methanol in dichloromethane). MS (ESI(−))m/e 685.6 [M+H]⁺; ¹H NMR (400 MHz, CDCl₃) δ 5.19-5.52 (m, 8H), 3.36-3.66 (m, 7H), 2.77 (t, J=6.14 Hz, 4H), 2.27-2.67 (m, 12H), 2.05 (q, J=6.75 Hz, 8H), 1.47-1.64 (m, 4H), 1.19-1.43 (m, 32H), 1.08 (t, J=7.21 Hz, 3H), 0.81-0.96 (m, 6H).

### Example 12

### N-(2,3-bis((9Z,12Z)-octadeca-9,12-dienyloxy)propyl)-N-methyl-N-(3-(pyrrolidin-1-ylmethyl)benzyl)amine

Prepared as described in EXAMPLE 11 by substituting N-methyl-1-(3-(pyrrolidin-1-ylmethyl)phenyl)methanamine for 1-ethylpiperazine. MS (ESI(−))m/e 775.5 [M+H]⁺; ¹H NMR (400 MHz, CDCl₃) δ 7.09-7.36 (m, 4H), 5.22-5.52 (m, 8H), 3.30-3.71 (m, 10H), 2.77 (t, J=6.44 Hz, 4H), 2.38-2.63 (m, 7H), 2.23 (s, 3H), 1.96-2.14 (m, 8H), 1.70-1.89 (m, 4H), 1.46-1.67 (m, 4H), 1.15-1.44 (m, 32H), 0.82-1.00 (m, 6H).

### Example 13

### N-(2-(4-(2,3-bis((9Z,12Z)-octadeca-9,12-dienyloxy)propyl)piperazin-1-yl)ethyl)-N,N-dimethylamine

Prepared as described in EXAMPLE 11 by substituting N,N-dimethyl-2-(piperazin-1-yl)ethanamine for 1-ethylpiperazine. MS (ESI(−))m/e 728.6 [M+H]⁺; ¹H NMR (400 MHz, CDCl₃) δ 5.23-5.48 (m, 8H) 3.35-3.67 (m, 7H) 2.77 (t, J=6.44 Hz, 4H) 2.38-2.63 (m, 15H) 2.19-2.31 (m, 6H) 2.05 (q, J=6.96 Hz, 8H) 1.47-1.63 (m, 4H) 1.18-1.45 (m, 32H) 0.81-0.97 (m, 6H).

### Example 14

### 1-((2S)-2,3-bis((9Z,12Z)-octadeca-9,12-dienyloxy)propyl)-4-methylpiperazine

Prepared as described in EXAMPLE 11 by substituting 1-methylpiperazine for 1-ethylpiperazine and (R)-3-chloropropane-1,2-diol for 3-chloropropane-1,2-diol. MS (ESI(−))m/e 671.7 [M+H]⁺; ¹H NMR (400 MHz, CDCl₃) δ 5.21-5.50 (m, 8H), 3.33-3.67 (m, 7H), 2.77 (t, J=6.14 Hz, 4H), 2.37-2.63 (m, 10H), 2.22-2.34 (m, 3H), 2.05 (q, J=6.65 Hz, 8H), 1.47-1.63 (m, 4H), 1.19-1.43 (m, 32H), 0.89 (t, J=6.90 Hz, 6H).

### Example 15

### 1-((2R)-2,3-bis((9Z,12Z)-octadeca-9,12-dienyloxy)propyl)-4-methylpiperazine

Prepared as described in EXAMPLE 11 by substituting 1-methylpiperazine for 1-ethylpiperazine and (S)-3-chloropropane-1,2-diol for 3-chloropropane-1,2-diol. MS (ESI(−))m/e 671.7 [M+H]⁺; ¹H NMR (400 MHz, CDCl₃) δ 5.21-5.50 (m, 8H), 3.33-3.67 (m, 7H), 2.77 (t, J=6.14 Hz, 4H), 2.37-2.63 (m, 10H), 2.22-2.34 (m, 3H), 2.05 (q, J=6.65 Hz, 8H), 1.47-1.63 (m, 4H), 1.19-1.43 (m, 32H), 0.89 (t, J=6.90 Hz, 6H).

### Example 16

### 1-(2,3-bis((9Z,12Z)-octadeca-9,12-dienyloxy)propyl)-4-(2-pyrrolidin-1-ylethyl)piperazine

Prepared as described in EXAMPLE 11 by substituting 1-(2-(pyrrolidin-1-yl)ethyl)piperazine for 1-ethylpiperazine. MS (ESI(−))m/e 754.5 [M+H]⁺; ¹H NMR (400 MHz, CDCl₃) δ 5.23-5.50 (m, 8H), 3.34-3.69 (m, 7H), 2.77 (t, J=6.29 Hz, 4H), 2.37-2.69 (m, 16H), 2.05 (q, J=6.65 Hz, 8H), 1.69-1.89 (m, 6H), 1.45-1.64 (m, 4H), 1.17-1.42 (m, 32H), 0.89 (t, J=6.75 Hz, 6H).

### Example 17

### 2-(4-(2,3-bis((9Z,12Z)-octadeca-9,12-dienyloxy)propyl)piperazin-1-yl)pyrimidine

Prepared as described in EXAMPLE 11 by substituting 2-(piperazin-1-yl)pyrimidine for 1-ethylpiperazine. MS (ESI(−))m/e 735.5 [M+H]⁺; ¹H NMR (400 MHz, CDCl₃) δ 8.29 (d, J=4.60 Hz, 2H), 6.46 (t, J=4.76 Hz, 1H), 5.18-5.52 (m, 8H), 3.80 (t, J=4.91 Hz, 4H), 3.34-3.69 (m, 7H), 2.77 (t, J=6.29 Hz, 4H), 2.42-2.67 (m, 6H), 2.05 (q, J=6.65 Hz, 8H), 1.45-1.65 (m, 4H), 1.16-1.45 (m, 32H), 0.89 (t, J=6.60 Hz, 6H).

### Example 18

### 1-(2,3-bis((9Z,12Z)-octadeca-9,12-dienyloxy)propyl)-N,N-diethylpyrrolidin-3-amine

Prepared as described in EXAMPLE 11 by substituting N,N-diethylpyrrolidin-3-amine for 1-ethylpiperazine. MS (ESI(−))m/e 713.6 [M+H]⁺; ¹H NMR (400 MHz, CDCl₃) δ 5.23-5.47 (m, 8H), 3.26-3.67 (m, 8H), 2.35-2.93 (m, 16H), 1.99-2.16 (m, 8H), 1.47-1.62 (m, 4H), 1.16-1.45 (m, 32H), 0.95-1.11 (m, 6H), 0.79-0.94 (m, 6H).

### Example 19

### 1-{2,3-bis[(9Z,12Z)-octadeca-9,12-dienyloxy]propyl}-N,N-dimethylpyrrolidin-3-amine

Prepared as described in EXAMPLE 11 by substituting N,N-dimethylpyrrolidin-3-amine for 1-ethylpiperazine. MS (ESI(−))m/e 685.6 [M+H]⁺; ¹H NMR (400 MHz, CDCl₃) δ 5.20-5.52 (m, 8H) 3.34-3.67 (m, 8H) 2.77 (t, J=5.93 Hz, 12H) 2.13-2.34 (m, 6H) 1.95-2.11 (m, 8H) 1.44-1.78 (m, 4H) 1.16-1.41 (m, 32H) 0.75-0.98 (m, 6H).

### Example 20

### 1-((9Z,12Z)-octadeca-9,12-dienyloxy)-3-pyrrolidin-1-ylpropan-2-ol

A mixture of 3-(pyrrolidin-1-yl)propane-1,2-diol (150 mg) and linoleyl methane sulfonate (300 mg) in toluene (5 mL) was treated with 95% oily sodium hydride (50 mg), stirred at room temperature for 5 minutes, heated in a sealed vial at 100° C. for 2 hours, cooled to room temperature, quenched with methanol and mixed with ethyl acetate and water. The extract was washed with brine, dried over Na₂SO₄, filtered and concentrated. The concentrate was purified by flash chromatography on silica gel (0-15% methanol in dichloromethane). MS (ESI(−))m/e 394.2 [M+H]⁺; ¹H NMR (400 MHz, CDCl₃) δ 5.21-5.50 (m, 4H) 3.63-3.96 (m, 1H) 3.32-3.60 (m, 5H) 2.34-2.94 (m, 8H) 1.94-2.16 (m, 4H) 1.67-1.87 (m, 4H) 1.46-1.65 (m, 2H) 1.13-1.44 (m, 16H) 0.89 (t, J=6.90 Hz, 3H).

### Example 21

### 2-[(9Z,12Z)-octadeca-9,12-dienoyloxy]-1-(pyrrolidin-1-ylmethyl)ethyl (9Z,12Z)-octadeca-9,12-dienoate

To a mixture 3-(pyrrolidin-1-yl)propane-1,2-diol (200 mg, 1.38 mmol), 4-dimethylaminopyridine (42 mg, 0.34 mmol), and (9Z,12Z)-octadeca-9,12-dienoic acid (0.92 g, 3.3 mmol) in CH₂Cl₂ (6 mL) was added 1-ethyl-3-[3-(dimethylamino)propyl]-carbodiimide hydrochloride (0.5 g, 3.58 mmol). The resulting reaction was allowed to stir at room temperature overnight. The reaction was partitioned between water and ethyl acetate. The organic layer was collected, dried over MgSO₄, filtered, and reduced in vacuo. The residue was purified by flash chromatography on silica gel (0-25% methanol/dichloromethane). MS (ESI(+)) m/e 671 (M+H)⁺. ¹H NMR (400 MHz, CHLOROFORM-D) δ ppm 5.28-5.43 (m, 8H) 5.19-5.27 (m, 1H) 4.32-4.39 (m, 1H) 4.05-4.12 (m, 1H) 2.74-2.82 (m, 4H) 2.69-2.73 (m, 1H) 2.58-2.65 (m, 3H) 2.25-2.35 (m, 4H) 2.00-2.10 (m, 8H) 1.73-1.82 (m, 3H) 1.56-1.67 (m, 4H) 1.2-1.41 (m, 31H) 0.85-0.94 (m, 6H).

### Example 22

### 2-[(9Z,12Z)-octadeca-9,12-dienyloxy]-1-(pyrrolidin-1-ylmethyl)ethyl (9Z,12Z)-octadeca-9,12-dienoate

### Example 22A

### 1-((9Z,12Z)-octadeca-9,12-dienyloxy)-3-(pyrrolidin-1-yl)propan-2-ol

To a solution of 3-(pyrrolidin-1-yl)propane-1,2-diol (1.5 equivalents), was added NaH (3 equivalents in toluene, followed by addition of (9Z,12Z)-octadeca-9,12-dienyl methanesulfonate (1 equivalent). The reaction was heated to 90° C. and stirred for 12 hours. The reaction was cooled, partitioned with brine and ethyl acetate. The organics were collected, and dried over MgSO₄, filtered, concentrated, and carried on to the next step without further purification.

### Example 22B

### 2-[(9Z,12Z)-octadeca-9,12-dienyloxy]-1-(pyrrolidin-1-ylmethyl)ethyl (9Z,12Z)-octadeca-9,12-dienoate

To a solution of EXAMPLE 22A (50 mg, 0.13 mmol) in CH₂Cl₂ (0.5 mL), was added (9Z,12Z)-octadeca-9,12-dienoic acid (54 mg, 0.19 mmol), 4-dimethylaminopyridine (2 mg, 0.02 mmol) and 1-ethyl-3-[3-(dimethylamino)propyl]-carbodiimide hydrochloride (35.5 mg, 0.229 mmol) and the mixture stirred at room temperature. After stirring at room temperature overnight, the reaction was partitioned between ethyl acetate and water. The organics were isolated, dried over MgSO₄, filtered and concentrated in vacuo. The residue was purified by flash chromatography on silica gel (5 to 10% methanol in methylene chloride). The desired fractions were concentrated, and the residue was purified by flash chromatography on silica gel (0-25% methanol/dichloromethane). MS (ESI(+)) m/e 657 (M+H)⁺. ¹H NMR (400 MHz, CHLOROFORM-D) δ ppm 5.26-5.43 (m, 8H) 5.13-5.22 (m, 1H) 3.49-3.55 (m, 1H) 3.35-3.48 (m, 2H) 2.72-2.81 (m, 5H) 2.62-2.71 (m, 3H) 2.24-2.36 (m, 3H) 1.99-2.10 (m, 8H) 1.74-1.84 (m, 2H) 1.48-1.68 (m, 5H) 1.20-1.42 (m, 32H) 0.84-0.95 (m, 6H).

### Example 231

### ({2-[(8Z,1Z)-heptadeca-8,11-dienyl]-2-[(9Z,12Z)-octadeca-9,12-dienyl]-1,3-dioxolan-4-yl}methyl)pyrrolidine

### Example 23A

### (6Z,9Z,27Z,30Z)-hexatriaconta-6,9,27,30-tetraen-18-one

To a suspension of magnesium (0.601 g, 24.71 mmol) in diethyl ether (11 ml) was added dropwise a solution of (6Z,9Z)-18-bromooctadeca-6,9-diene (3.7 g, 11.23 mmol) in diethyl ether (11 ml). The reaction mixture was refluxed for 1 hour then cooled to room temperature to give a solution of the Grignard reagent (9Z,12Z)-octadeca-9,12-dienylmagnesium bromide.

To a solution of (9Z,12Z)-octadeca-9,12-dienoic acid (3 g, 10.70 mmol) in CHCl₃ (5 ml) was added oxalyl chloride (1.124 ml, 12.84 mmol) and a drop of DMF. The reaction mixture was stirred at room temperature for 1 hour. The reaction mixture was concentrated by rotary evaporation. The residue was taken up in THF (5 ml) and cooled to −78° C. A solution of (9Z,12Z)-octadeca-9,12-dienylmagnesium bromide (10.70 ml, 10.70 mmol) was added dropwise. The reaction mixture was warmed to 0° C. The reaction mixture was quenched in cold water. The mixture was partitioned between water and ethyl acetate. The organic layer was dried over sodium sulfate, filtered, and concentrated. The crude material was purified by flash chromatography (4:1 hexanes/dichloromethane). MS (DCI) m/z 530.5 (M+18)⁺.

### Example 23B

To a solution of 3-(pyrrolidin-1-yl)propane-1,2-diol (28.3 mg, 0.195 mmol) in toluene, was added (6Z,9Z,27Z,30Z)-hexatriaconta-6,9,27,30-tetraen-18-one (100 mg, 0.19 mmol), followed by 4-methylbenzenesulfonic acid (3.3 mg, 0.02 mmol) and heated to 80° C. The reaction was cooled to room temperature diluted with saturated aqueous NaHCO₃ and chloroform. The organics were separated, dried over MgSO₄, filtered, and reduced in vacuo. The residue was concentrated, and the concentrate was purified by flash chromatography on silica gel (0-25% methanol/dichloromethane). MS (ESI(+)) m/e 640 (M+H)⁺. ¹H NMR (300 MHz, CHLOROFORM-D) δ ppm 5.27-5.45 (m, 8H) 3.54 (t, 1H) 2.74-2.81 (m, 4H) 2.51-2.72 (m, 4H) 1.99-2.09 (m, 8H) 1.77-1.87 (m, 3H) 1.53-1.65 (m, 10H) 1.22-1.40 (m, 33H) 0.84-0.93 (m, 6H).

### Example 24

### 1-{2,3-bis[(5Z,8Z,11Z,14Z)-icosa-5,8,11,14-tetraenyloxy]propyl}pyrrolidine

EXAMPLE 24 was prepared following the procedure detailed for EXAMPLE 1 in which linoleyl methane sulfonate was replaced with (5Z,8Z,11Z,14Z)-icosa-5,8,11,14-tetraenyl methane sulfonate. MS (ESI(+)) m/e 691 (M+H)⁺. ¹H NMR (400 MHz, CHLOROFORM-D) δ ppm 5.29-5.44 (m, 16H) 4.22 (t, 1H) 3.48-3.64 (m, 5H) 3.40-3.48 (m, 3H) 2.98-3.00 (m, 1H) 2.77-2.86 (m, 13H) 2.64-2.72 (m, 1H) 2.47-2.62 (m, 6H) 2.01-2.19 (m, 9H) 1.72-1.80 (m, 5H) 1.54-1.64 (m, 4H) 1.23-1.52 (m, 10H) 0.86-0.92 (m, 6H).

### Example 25

### 1-{3-[(5Z,8Z,11Z,14Z)-icosa-5,8,11,14-tetraenyloxy]-2-[(9Z,12Z)-octadeca-9,12-dienyloxy]propyl}pyrrolidine

EXAMPLE 25 was prepared by taking EXAMPLE 22A and treating it with (5Z,8Z,11Z,14Z)-icosa-5,8,11,14-tetraenyl methane sulfonate. MS (ESI(+)) m/e 667 (M+H)⁺.

### Example 26

### 1-{2,3-bis[(9E,12E)-octadeca-9,12-dienyloxy]propyl}pyrrolidine

### Example 27

### 1-{2-[(9E,12E)-octadeca-9,12-dienyloxy]-3-[(9Z,12Z)-octadeca-9,12-dienyloxy]propyl}pyrrolidine

To EXAMPLE 22A (0.19 g, 0.49 mmol) in toluene was added NaH (0.046 g, 1.94 mmol), and the solution was allowed to stir at room temperature for 15 minutes. (9E,12E)-octadeca-9,12-dienyl methanesulfonate (0.5 g, 1.45 mmol) was added and the reaction was heated to 90° C. The reaction was allowed to stir at 90° C. overnight. The reaction was cooled to room temperature and diluted with ethyl acetate and treated with water. The organics were collected, dried over MgSO₄, filtered, and concentrated in vacuo. The residue was concentrated, and the concentrate was purified by flash chromatography on silica gel (0-25% methanol/dichloromethane). MS (ESI(+)) m/e 643 (M+H)⁺. ¹H NMR (300 MHz, CHLOROFORM-D) δ ppm 5.28-5.45 (m, 8H) 3.47-3.64 (m, 4H) 3.39-3.48 (m, 3H) 2.75-2.81 (m, 2H) 2.62-2.70 (m, 3H) 2.43-2.59 (m, 5H) 1.92-2.11 (m, 8H) 1.71-1.80 (m, 4H) 1.49-1.64 (m, 6H) 1.21-1.41 (m, 30H) 0.83-0.93 (m, 6H).

### Example 28

### 1-[2,3-bis(tetradecyloxy)propyl]pyrrolidine

EXAMPLE 28 was prepared following the procedure detailed for EXAMPLE 1 in which linoleyl methane sulfonate was replaced with tetradecyl methanesulfonate. MS (ESI(+)) m/e 539 (M+H)⁺. ¹H NMR (400 MHz, CHLOROFORM-D) δ ppm 3.63-3.71 (m, 1H) 3.54-3.63 (m, 1H) 3.48-3.54 (m, 2H) 3.40-3.47 (m, 4H) 2.53-2.81 (m, 6H) 1.76-1.85 (m, 4H) 1.50-1.60 (m, 4H) 1.19-1.36 (m, 43H) 0.83-0.91 (m, 6H).

### Example 29

### 1-[2,3-bis(octadecyloxy)propyl]pyrrolidine

EXAMPLE 29 was prepared following the procedure detailed for EXAMPLE 1 in which linoleyl methane sulfonate was replaced with octadecyl methanesulfonate. MS (ESI(+)) m/e 651 (M+H). ¹H NMR (400 MHz, CHLOROFORM-D) δ ppm 3.48-3.62 (m, 4H) 3.41-3.47 (m, 3H) 2.61-2.68 (m, 1H) 2.46-2.60 (m, 5H) 1.72-1.77 (m, 4H) 1.61-1.68 (m, 2H) 1.51-1.60 (m, 4H) 1.21-1.35 (m, 58H) 0.85-0.91 (m, 6H).

### Example 30

### 1-{2,3-bis[(9Z)-octadec-9-enyloxy]propyl}pyrrolidine

EXAMPLE 30 was prepared following the procedure detailed for EXAMPLE 1 in which linoleyl methane sulfonate was replaced with oleyl methane sulfonate. MS (ESI(+)) m/e 651 (M+H)⁺. ¹H NMR (400 MHz, CHLOROFORM-D) δ ppm 5.29-5.40 (m, 4H) 3.47-3.62 (m, 4H) 3.39-3.47 (m, 3H) 2.61-2.69 (m, 1H) 2.44-2.60 (m, 5H) 1.93-2.06 (m, 8H) 1.69-1.79 (m, 4H) 1.50-1.61 (m, 4H) 1.18-1.39 (m, 44H) 0.83-0.93 (m, 6H).

### Example 31

### 1-[2,3-bis(dodecyloxy)propyl]pyrrolidine

### Example 321

### {2,3-bis[(9Z,12Z)-octadeca-9,12-dienyloxy]propyl}pyrrolidin-3-ol

### Example 32A

### 3-(bis(4-methoxyphenyl)(phenyl)methoxy)propane-1,2-diol

To a solution of glycerol (0.431 ml, 5.90 mmol) in pyridine (5 ml) at 0° C. was added 4,4′-(chloro(phenyl)methylene)bis(methoxybenzene) (2 g, 5.90 mmol). The reaction was stirred at room temperature for 4 hours. The solvent was removed under vacuum. The residue was partitioned between water and ethyl acetate. The aqueous layer was extracted with ethyl acetate. The combined organics were washed with water and brine, dried over sodium sulfate, filtered, and concentrated. The crude material was purified by flash chromatography (1:1 hexanes/ethyl acetate, 2% triethylamine, 2-10% methanol) to give the title compound.

### Example 32B

### 4,4′-((2,3-bis((9Z,12Z)-octadeca-9,12-dienyloxy)propoxy)(phenyl)methylene)bis(methoxybenzene)

To a solution of EXAMPLE 22A (0.189 g, 0.479 mmol) in toluene (2.4 ml) at 0° C. was added sodium hydride (0.097 g, 3.83 mmol). The reaction mixture was stirred at room temperature for 30 minutes then recooled to ° C. (9Z,12Z)-octadeca-9,12-dienyl methanesulfonate (0.413 g, 1.198 mmol) was added. The reaction mixture was heated at reflux (120° C.) for 2 hours. The reaction mixture was cooled to 0° C. and quenched with ethanol. The mixture was concentrated by rotary evaporation. The residue was taken up in dichloromethane and dried onto silica gel. The crude material was purified by flash chromatography (2-5% ethyl acetate/hexanes) to give the title compound. MS (ESI(+)) m/e 423 (M+H)⁺.

### Example 32C

### 2,3-bis((9Z,12Z)-octadeca-9,12-dienyloxy)propan-1-ol

To a solution of EXAMPLE 22B (0.240 g, 0.269 mmol) in dichloromethane (2.25 ml) at 0° C. was added 2,2-dichloroacetic acid (0.078 ml, 0.942 mmol). The reaction mixture was stirred at room temp for 1 hour, followed by addition of 0.078 mL of dichloroacetic acid. The reaction mixture was stirred at room temperature for 1 hour, then cooled to 0° C. and quenched with water. The solution was adjusted to pH 4-5 using saturated aqueous NaHCO₃. The layers were separated, and the aqueous layer was extracted with dichloromethane. The combined organics were dried over sodium sulfate, filtered, and concentrated. The crude material was purified by flash chromatography (10% ethyl acetate/hexanes) to give the title compound. MS (ESI(+)) m/e 607 (M+H)⁺.

### Example 32D

### 2,3-bis((9Z,12Z)-octadeca-9,12-dienyloxy)propanal

To a solution of oxalyl chloride (0.21 mL, 2.45 mmol) in CH₂Cl₂ (5 mL) at −78° C., dimethyl sulfoxide (0.20 mmol, 2.4 mmol) was added dropwise. The solution was stirred for an additional 5 minutes. EXAMPLE 22C (1.2 g, 2.0 mmol) in CH₂Cl₂ (5 mL) was added dropwise. After stirring at −78° C. for 30 minutes, triethylamine (0.83 mL, 6.1 mmol) was added dropwise and the mixture stirred for 15 minutes. The reaction was allowed to warm to room temperature. The reaction was diluted with CH₂Cl₂ and water. The organics were separated. The aqueous layer was extracted with CH₂Cl₂ (2×20 mL). The organics were combined, dried over MgSO₄, filtered and concentrated in vacuo.

### Example 32E

### 1-(2,3-bis((9Z,12Z)-octadeca-9,12-dienyloxy)propyl)pyrrolidin-3-ol

To a solution of EXAMPLE 22D (0.80 g, 1.37 mmol) in tetrahydrofuran (2 ml) were added pyrrolidin-3-ol (0.14 g, 1.64 mmol) and acetic acid (0.25 g, 4.1 mmol). The reaction mixture was stirred at room temperature for 1 hour. The reaction mixture was cooled to 0° C. Sodium triacetoxyborohydride (0.58 g, 2.73 mmol) was added, and the reaction mixture was stirred at room temp for 4 hours. The reaction was partitioned between water and ethyl acetate. The aqueous layer was extracted with ethyl acetate (3×). The combined organics were washed with saturated aqueous NaHCO₃, water, and brine, dried over Na₂SO₄, filtered, and concentrated. The crude material was dissolved in dichloromethane and dried onto silica gel. The silica was loaded into an Analogix DASI module, and the product was isolated by flash chromatography (Analogix, SF25×60 g, 1% methanol/ethyl acetate for six column volumes, then 1-10% methanol/ethyl acetate over six column volumes) to give the title compound. MS (ESI(+)) m/e 607 (M+H)⁺. ¹H NMR (400 MHz, CHLOROFORM-D) δ ppm 5.28-5.42 (m, 8H) 4.25-4.32 (m, 1H) 3.40-3.62 (m, 8H) 2.91-3.00 (m, 1H) 2.73-2.82 (m, 5H) 2.61-2.70 (m, 1H) 2.50-2.59 (m, 2H) 2.26-2.38 (m, 3H) 2.10-2.21 (m, 1H) 1.99-2.09 (m, 7H) 1.67-1.79 (m, 1H) 1.50-1.60 (m, 4H) 1.23-1.40 (m, 32H) 0.87-0.93 (m, 6H).

### Example 33

### 1-{3-[(9Z,12Z)-hexadeca-9,12-dienyloxy]-2-[(9Z)-octadec-9-enyloxy]propyl}pyrrolidine

EXAMPLE 33 was prepared as described in EXAMPLE 27, replacing (9E,12E)-octadeca-9,12-dienyl methanesulfonate with (Z)-octadec-9-enyl methanesulfonate. ¹H NMR (400 MHz, CHLOROFORM-D) δ ppm 5.28-5.43 (m, 6H) 3.47-3.64 (m, 4H) 3.39-3.48 (m, 3H) 2.74-2.81 (m, 2H) 2.62-2.71 (m, 1H) 2.45-2.60 (m, 5H) 1.97-2.11 (m, 9H) 1.71-1.79 (m, 4H) 1.51-1.61 (m, 4H) 1.22-1.41 (m, 34H) 0.84-0.93 (m, 5H).

### Example 34

### 1-[3-[(9Z,12Z)-hexadeca-9,12-dienyloxy]-2-(tetradecyloxy)propyl]pyrrolidine

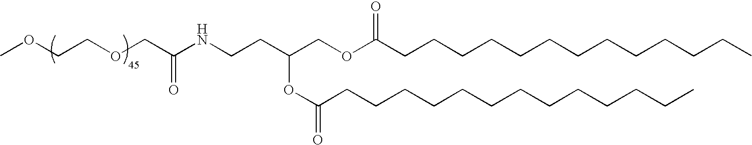

### Example 35

### 6-oxo-2-(tetradecanoyloxy)-8,11,14,17,20,23,26,29,32,35,38,41,44,47,50,53,56,59,62,65,68,71,74,77,80,83,86,89,92,95,98,101,104,107,110,113,116,119,122,125,128,131,134,137,140,143-hexatetracontaoxa-5-a zatetratetracontahect-1-yl myristate

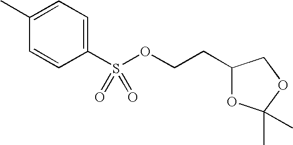

### Example 35A

### 2-(2,2-dimethyl-1,3-dioxolan-4-yl)ethyl 4-methylbenzenesulfonate

2-(2,2-Dimethyl-1,3-dioxolan-4-yl)ethanol (5 g) was added to dichloromethane (86 ml) and the mixture was cooled to 0° C. To this solution was added triethylamine (6.9 g, 9.6 ml), tosyl chloride (6.5 g) and 4-(dimethylamino)pyridine (0.42 g). The mixture stirred at room temperature overnight. The mixture was quenched with saturated NH₄Cl and diluted with ethyl acetate. The aqueous layer was extracted twice with ethyl acetate and the extract was dried (Na₂SO₄), filtered, and concentrated. The concentrate was purified by flash column chromatography (Analogix hexanes:ethyl acetate, 0-75%) to afford the title compound. MS (ESI) m/z 300.9 (M+H)⁺; ¹H NMR (400 MHz, CDCl₃) δ 7.79 (d, J=8.29 Hz, 2H) 7.35 (d, J=7.98 Hz, 2H) 4.06-4.23 (m, 3H) 4.01 (dd, J=7.98, 6.14 Hz, 1H) 3.51 (dd, J=8.13, 6.90 Hz, 1H) 2.45 (s, 3H) 1.82-1.98 (m, 2H) 1.31 (d, J=18.72 Hz, 6H).

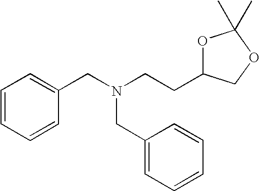

### Example 35B

### N,N-dibenzyl-2-(2,2-dimethyl-1,3-dioxolan-4-yl)ethanamine

EXAMPLE 35A (1.0 g) and dibenzylamine (0.657 mg) were placed in a microwave vial (Biotage) and dioxane (2.5 mL) was added. The vial was capped and placed in a microwave reactor (Biotage Initiator), and the mixture was heated at 150° C. for 30 minutes. The mixture was diluted with ethyl acetate and poured into water. The aqueous layer was extracted twice with ethyl acetate, and the extract was washed with brine, dried (Na₂SO₄), filtered and concentrated. The concentrate was used in the next step without further purification.

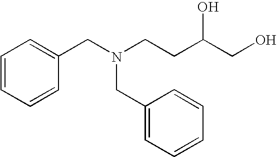

### Example 35C

### 4-(dibenzylamino)butane-1,2-diol

EXAMPLE 35B was added to tetrahydrofuran (20 mL) and 2N HCl (20 mL), and the mixture was stirred at room temperature for 30 minutes. 5N NaOH was added until the solution was basic, and the aqueous layer was extracted with chloroform. The extract was dried (MgSO₄), filtered and concentrated by rotary evaporation and the concentrate was used in the next step without further purification. MS (ESI) m/z 285.9 (M+H)⁺.

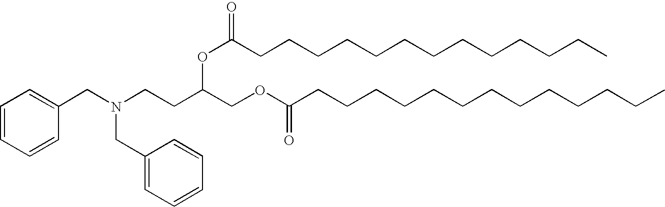

### Example 35D

### 4-(dibenzylamino)butane-1,2-diyl ditetradecanoate

A mixture of EXAMPLE 35C (700 mg), tetradecanoic acid (1.68 g), N¹-((ethylimino)methylene)-N³, N³-dimethylpropane-1,3-diamine hydrochloride (1.41 g) and 4-(dimethylamino)pyridine (45 mg) in dichloromethane (5 mL) was heated at 40° C. until the mixture was homogenous and then was stirred overnight at room temperature. Water was added along with some brine and the aqueous layer was extracted with dichloromethane (3×). The extract was dried (Na₂SO₄), filtered and the filtrate was concentrated. The concentrate was purified by flash column chromatography (Analogix 280, 0-50% ethyl acetate/hexanes) to provide the title compound. MS (ESI) m/z 706.5 (M+H)⁺; ¹H NMR (300 MHz, CDCl₃) δ 7.16-7.39 (m, 10H) 5.06-5.21 (m, 1H) 4.12 (dd, J=11.70, 3.37 Hz, 1H) 3.91 (dd, J=11.90, 5.95 Hz, 1H) 3.41-3.62 (m, 4H) 2.35-2.57 (m, 2H) 2.25 (t, J=7.54 Hz, 2H) 2.02-2.19 (m, 2H) 1.77 (q, J=7.40 Hz, 2H) 1.45-1.63 (m, 4H) 1.17-1.36 (m, 40H) 0.82-0.94 (m, 6H).

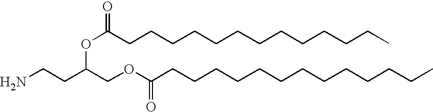

### Example 35E

### 4-aminobutane-1,2-diyl ditetradecanoate

EXAMPLE 35D (500 mg) was added to methanol/dichloromethane/ethyl acetate (1/1/1, 10 mL) and combined with catalytic Pd/C (10%). Hydrogen was introduced via a balloon, and the mixture was stirred overnight then filtered through Celite®. The filtrate was concentrated and the concentrate was used in the next step without further purification. MS (ESI) m/z 526.6 (M+H)⁺; ¹H NMR (300 MHz, CDCl₃) δ ppm 5.13-5.25 (m, 1H) 4.02-4.35 (m, 2H) 2.91-3.23 (m, 2H) 2.24-2.42 (m, 4H) 1.97-2.23 (m, 2H) 1.44-1.73 (m, 6H) 1.26 (s, 40H) 0.81-0.96 (m, 6H).

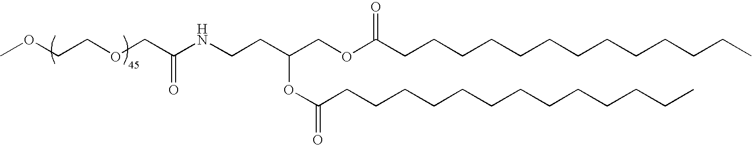

### Example 35F

### 6-oxo-2-(tetradecanoyloxy)-8,11,14,17,20,23,26,29,32,35,38,41,44,47,50,53,56,59,62,65,68,71,74,77,80,83,86,89,92,95,98,101,104,107,110,113,116,119,122,125,128,131,134,137,140,143-hexatetracontaoxa-5-a zatetratetracontahect-1-yl myristate

mPEG2000-SCM (139 mg, Laysan Bio, Inc) and EXAMPLE 35E (100 mg) were combined in a 4 mL vial with dichloromethane (1 mL) and triethylamine (26.5 μL). The mixture was stirred at room temperature overnight. The mixture was loaded directly onto a silica gel column (Analogix) and eluted with dichlormethane/methanol (0-20%). MS (MALDI) m/z 2690.5; ¹H NMR (300 MHz, CDCl₃) δ ppm 5.07-5.20 (m, 1H) 4.24 (dd, J=11.90, 3.17 Hz, 1H) 4.06 (dd, J=11.90, 6.35 Hz, 1H) 3.98 (s, 2H) 3.85-3.91 (m, 1H) 3.61-3.70 (m, 29H) 3.39-3.59 (m, 6H) 3.38 (s, 3H) 3.14-3.30 (m, 1H) 2.25-2.36 (m, 4H) 1.53-1.87 (m, 6H) 1.26 (s, 40H) 0.83-0.93 (m, 6H).

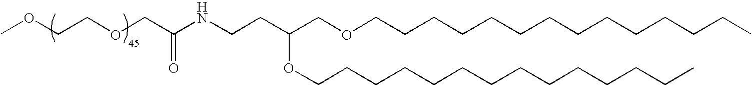

### Example 36

### N-[3,4-bis(tetradecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanon atriacontahectan-139-amide

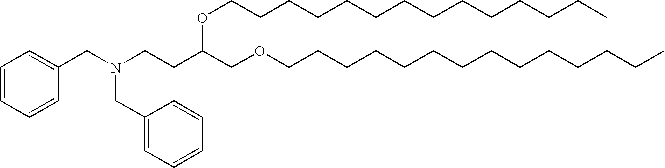

### Example 36A

### N,N-dibenzyl-3,4-bis(tetradecyloxy)butan-1-amine

EXAMPLE 35C (1 g) in toluene (6 mL) and added to NaH (0.336 g, dry, 95%) in toluene (6 mL). The mixture was stirred at room temperature for 1 hour. Tetradecyl methanesulfonate (2.15 g) was added. The mixture was heated to 90° C. overnight. The mixture was cooled to room temperature and ethanol was added followed by water until the excess NaH was destroyed. The mixture was poured into water and brine and extracted with ethyl acetate. The water was extracted with ethyl acetate, and the extract was dried (Na₂SO₄), filtered and concentrated. The concentrate was purified by an Analogix system (hexane:ethyl acetate, 0-50%)). MS (ESI) m/z 678.6 (M+H)⁺; ¹H NMR (300 MHz, CDCl₃) δ ppm 7.16-7.40 (m, 10H) 3.14-3.63 (m, 11H) 2.44-2.59 (m, 2H) 1.59-1.82 (m, 2H) 1.35-1.53 (m, 4H) 1.14-1.34 (m, 44H) 0.82-0.94 (m, 6H).

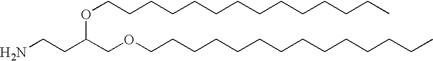

### Example 36B

### 3,4-bis(tetradecyloxy)butan-1-amine

EXAMPLE 36B was prepared using the procedure described for EXAMPLE 35E, substituting EXAMPLE 36A for EXAMPLE 35D. MS (ESI) m/z 498.5 (M+H)⁺; ¹H NMR (300 MHz, CDCl₃) δ ppm 8.24 (s, 2H) 3.53-3.70 (m, 1H) 3.34-3.53 (m, 6H) 3.07-3.34 (m, 2H) 1.87-2.13 (m, 2H) 1.48-1.67 (m, 4H) 1.16-1.39 (m, 44H) 0.82-0.94 (m, 6H).

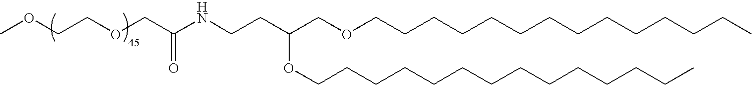

### Example 36C

### N-[3,4-bis(tetradecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanon atriacontahectan-139-amide

EXAMPLE 36C was prepared using the procedure described for EXAMPLE 35F, substituting EXAMPLE 26B for EXAMPLE 35E. MS (MALDI) m/z 2617.6; ¹H NMR (300 MHz, CDCl₃) δ 3.95-4.02 (m, 2H) 3.83-3.92 (m, 1H) 3.68-3.72 (m, 1H) 3.65 (m, 180H) 3.35-3.60 (m, 10H) 1.59-1.73 (m, 2H) 1.49-1.60 (m, 4H) 1.18-1.36 (m, 44H) 0.82-0.94 (m, 6H).

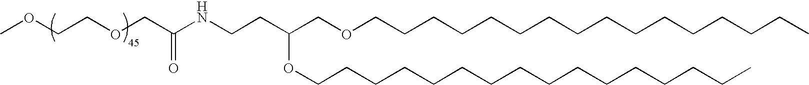

### Example 37

### N-[3,4-bis(hexadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanona triacontahectan-139-amide

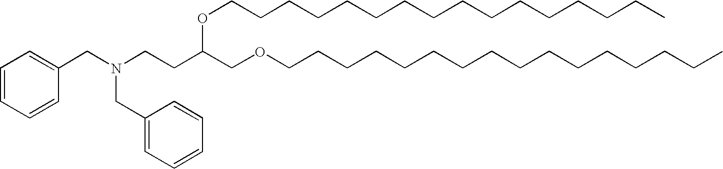

### Example 37A

### N,N-dibenzyl-3,4-bis(hexadecyloxy)butan-1-amine

EXAMPLE 37A was prepared using the procedure described for EXAMPLE 36A, substituting hexadecyl methanesulfonate for tetradecyl methanesulfonate. MS (ESI) m/z 734.6 (M+H)⁺; ¹H NMR (300 MHz, CDCl₃) δ ppm 7.15-7.41 (m, 10H) 3.12-3.64 (m, 11H) 2.41-2.64 (m, 2H) 1.35-1.80 (m, 6H) 1.15-1.34 (m, 52H) 0.81-0.94 (m, 6H).

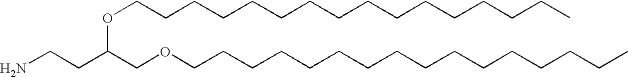

### Example 37B

### 3,4-bis(hexadecyloxy)butan-1-amine

EXAMPLE 37B was prepared using the procedure described for EXAMPLE 36B, substituting EXAMPLE 37A for EXAMPLE 36A. MS (ESI) m/z 554.6 (M+H)⁺; ¹H NMR (300 MHz, CDCl₃) δ ppm 8.12-8.38 (m, 2H) 3.54-3.70 (m, 1H) 3.33-3.53 (m, 6H) 3.06-3.33 (m, 2H) 1.84-2.14 (m, 2H) 1.46-1.71 (m, 4H) 1.14-1.37 (m, 52H) 0.81-0.94 (m, 6H).

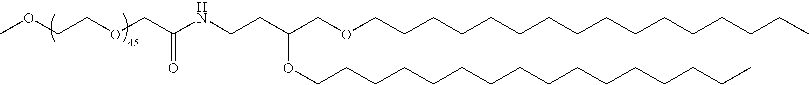

### Example 37C

### N-[3,4-bis(hexadecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanona triacontahectan-139-amide

EXAMPLE 37C was prepared using the procedure described for EXAMPLE 35F, substituting EXAMPLE 37B for EXAMPLE 35E. MS (MALDI) m/z 2866.7; ¹H NMR (300 MHz, CDCl₃) δ ppm 3.98 (s, 2H) 3.84-3.91 (m, 1H) 3.60-3.68 (m, 180H) 3.36-3.60 (m, 11H) 1.50-1.72 (m, 6H) 1.26 (s, 52H) 0.84-0.92 (m, 6H).

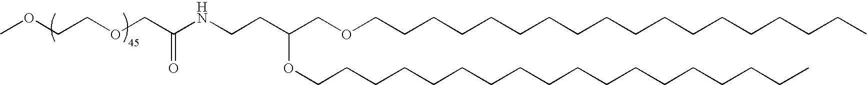

### Example 38

### N-[3,4-bis(octadecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanona triacontahectan-139-amide

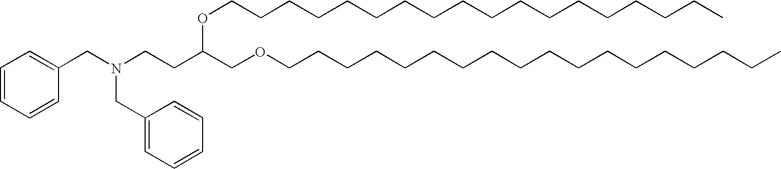

### Example 38A

### N,N-dibenzyl-3,4-bis(octadecyloxy)butan-1-amine

EXAMPLE 558A was prepared using the same procedure described for EXAMPLE 36A, substituting octadecyl methanesulfonate for tetradecyl methanesulfonate. LCMS (APCI) m/z 790.6; ¹H NMR (300 MHz, CDCl₃) δ ppm 7.15-7.41 (m, 10H) 3.10-3.68 (m, 11H) 2.39-2.68 (m, 2H) 1.35-1.80 (m, 6H) 1.14-1.34 (m, 60H) 0.81-0.94 (m, 6H).

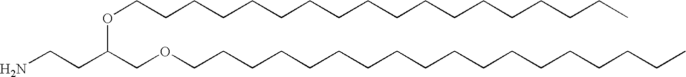

### Example 38B

### 3,4-bis(octadecyloxy)butan-1-amine

EXAMPLE 38B was prepared using the same procedure described for EXAMPLE 35E, substituting EXAMPLE 38A for EXAMPLE 35D. LCMS (APCI) m/z 610.9; ¹H NMR (300 MHz, CDCl₃) δ 3.08-3.70 (m, 9H) 1.85-2.15 (m, 2H) 1.55 (s, 4H) 1.15-1.37 (m, 60H) 0.84-0.92 (m, 6H).

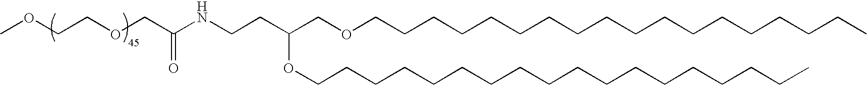

### Example 38C

### N-[3,4-bis(octadecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanona triacontahectan-139-amide

EXAMPLE 38C was prepared using the same procedure described for EXAMPLE 35F, substituting EXAMPLE 38B for EXAMPLE 35E. MS (MALDI) m/z 2773.6; ¹H NMR (300 MHz, CDCl₃) δ ppm 3.95-4.01 (m, 2H) 3.84-3.91 (m, 1H) 3.59-3.70 (m, 180H) 3.27-3.59 (m, 11H) 1.49-1.86 (m, 6H) 1.18-1.35 (m, 60H) 0.80-0.94 (m, 6H).

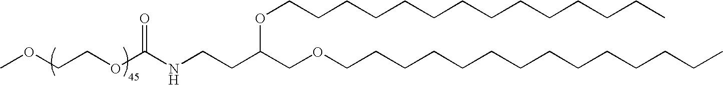

### Example 39

### 3,7,11,15,19,23,27,31,35,39,43,47,51,55,59,63,67,71,75,79,83,87,91,95,99,103,107,111,115,119,123,127,131,135,139,143,147,151,155,159,163,167,171,175,179,182-hexatetracontaoxatrioctacontahect-1-yl 3,4-bis(tetradecyloxy)butylcarbamate

EXAMPLE 36B (100 mg) was dissolved in dichloromethane (1-2 mL) and mPEG-NPC (26.0 mg) was added. Hunig's base (26 mg) was added, and the mixture was stirred overnight at room temperature. The mixture was loaded directly onto a silica gel column (4 g Analogix) and chromatographed (Analogix 280, dichloromethane/methanol, 0-20%) to give Example 39. MS (MALDI) m/z 2472.2; ¹H NMR (300 MHz, CDCl₃) δ ppm 4.16-4.24 (m, 2H) 3.78-3.92 (m, 1H) 3.59-3.70 (m, 180H) 3.52-3.61 (m, 4H) 3.19-3.49 (m, 9H) 1.48-1.82 (m, 6H) 1.21-1.35 (m, 44H) 0.82-0.93 (m, 6H).

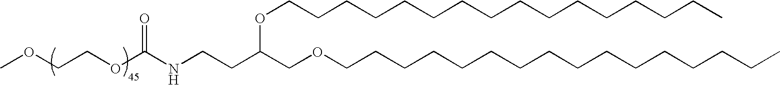

### Example 40

### 3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hexatetracontaoxanonatriacontahect-1-yl 3,4-bis(hexadecyloxy)butylcarbamate

EXAMPLE 40 was prepared using the same procedure described for EXAMPLE 39, substituting EXAMPLE 37B for EXAMPLE 36B. MS (MALDI) m/z 2395.0; ¹H NMR (300 MHz, CDCl₃) δ ppm 4.15-4.23 (m, 2H) 3.81-3.92 (m, 1H) 3.60-3.71 (m, 180H) 3.47-3.59 (m, 4H) 3.33-3.48 (m, 9H) 1.48-1.81 (m, 6H) 1.19-1.34 (m, 52H) 0.83-0.92 (m, 6H).

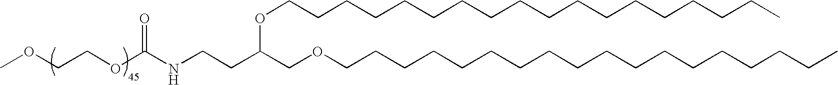

### Example 41

### 3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hexatetracontaoxanonatriacontahect-1-yl 3,4-bis(octadecyloxy)butylcarbamate

EXAMPLE 41 was prepared using the same procedure described for EXAMPLE 39, substituting EXAMPLE 38B for EXAMPLE 36B. MS (MALDI) m/z 2495.8; ¹H NMR (300 MHz, CDCl₃) δ ppm 4.16-4.24 (m, 2H) 3.82-3.92 (m, 1H) 3.60-3.71 (m, 180H) 3.49-3.59 (m, 4H) 3.17-3.49 (m, 9H) 1.48-1.80 (m, 6H) 1.18-1.37 (m, 60H) 0.82-0.93 (m, 6H).

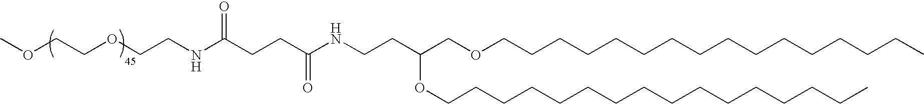

### Example 42

### N-[3,4-bis(hexadecyloxy)butyl]-N′-3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hexatetracontaoxan onatriacontahect-1-ylsuccinamide

EXAMPLE 42 was prepared using the same procedure described for EXAMPLE 35F, substituting RAPP 12 2000-35 (Rapp Polymere) for mPEG2000-SCM. MS (MALDI) m/z 2584.3; ¹H NMR (300 MHz, CDCl₃) δ ppm 6.43-6.61 (m, 2H) 3.60-3.68 (m, 200H) 3.36-3.58 (m, 16H) 2.42-2.57 (m, 4H) 1.49-1.85 (m, 6H) 1.19-1.35 (m, 52H) 0.82-0.92 (m, 6H).

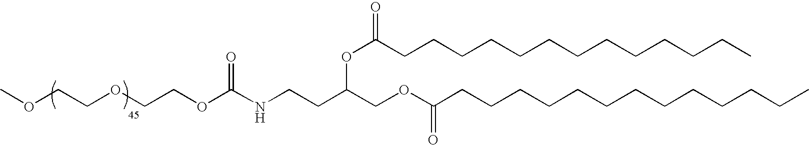

### Example 43

### 6-oxo-2-(tetradecanoyloxy)-7,10,13,16,19,22,25,28,31,34,37,40,43,46,49,52,55,58,61,64,67,70,73,76,79,82,85,88,91,94,97,100,103,106,109,112,115,118,121,124,127,130,133,136,139,142,145-heptatetracontaox a-5-azahexatetracontahect-1-yl myristate

EXAMPLE 43 was prepared using the same procedure described for EXAMPLE 35F, substituting mPEG-NPC (Creative PEGWorks) for mPEG2000-SCM (Laysan Bio, Inc.). MS (MALDI) m/z 2588.5; ¹H NMR (300 MHz, CDCl₃) δ ppm 5.14 (m, 1H) 4.17-4.26 (m, 3H) 4.01-4.11 (m, 1H) 3.83-3.91 (m, 1H) 3.60-3.71 (m, 180H) 3.48-3.60 (m, 4H) 3.35-3.44 (m, 5H) 2.23-2.37 (m, 4H) 1.62-1.86 (m, 6H) 1.21-1.37 (m, 40H) 0.83-0.93 (m, 6H).

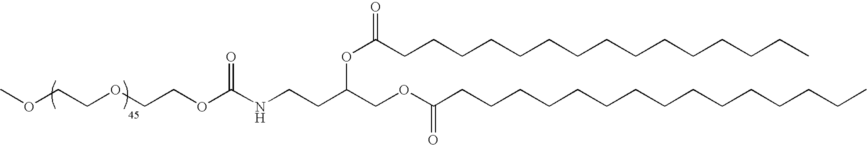

### Example 44

### 6-oxo-2-(palmitoyloxy)-7,10,13,16,19,22,25,28,31,34,37,40,43,46,49,52,55,58,61,64,67,70,73,76,79,82,85,88,91,94,97,100,103,106,109,112,115,118,121,124,127,130,133,136,139,142,145-heptatetracontaoxa-5-azahexatetracontahect-1-yl palmitate

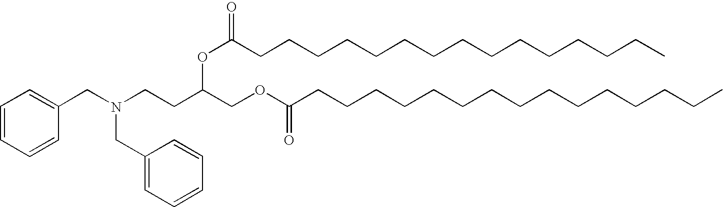

### Example 44A

### 4-(dibenzylamino)butane-1,2-diyl dipalmitate

EXAMPLE 44A was prepared using the same procedure described for EXAMPLE 35D, substituting hexadecanoic acid for tetradecanoic acid. MS (ESI) m/z 762.4 (M+H)⁺; ¹H NMR (300 MHz, CDCl₃) δ ppm 7.15-7.42 (m, 10H) 5.06-5.21 (m, 1H) 4.12 (dd, J=11.90, 3.57 Hz, 1H) 3.91 (dd, J=11.90, 5.95 Hz, 1H) 3.43-3.62 (m, 4H) 2.34-2.58 (m, 2H) 2.25 (t, J=7.34 Hz, 2H) 2.01-2.16 (m, 2H) 1.77 (q, J=7.14 Hz, 2H) 1.40-1.64 (m, 4H) 1.14-1.37 (m, 48H) 0.82-0.95 (m, 6H).

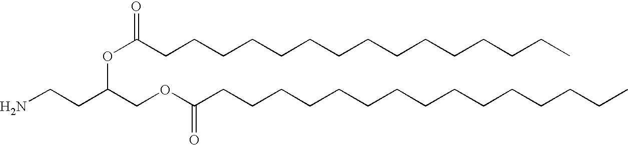

### Example 44B

### 4-aminobutane-1,2-diyl dipalmitate

EXAMPLE 44B was prepared using the same procedure described for EXAMPLE 35E, substituting EXAMPLE 44A for EXAMPLE 35D. MS (ESI) m/z 482.6 (M+H)⁺.

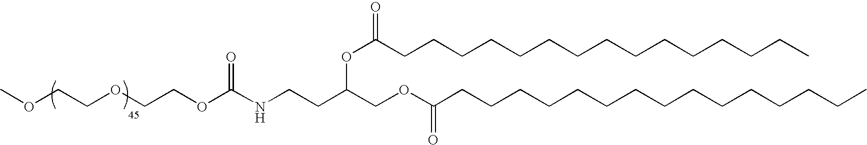

### Example 44C

### 6-oxo-2-(palmitoyloxy)-7,10,13,16,19,22,25,28,31,34,37,40,43,46,49,52,55,58,61,64,67,70,73,76,79,82,85,88,91,94,97,100,103,106,109,112,115,118,121,124,127,130,133,136,139,142,145-heptatetracontaoxa-5-azahexatetracontahect-1-yl palmitate

EXAMPLE 44C was prepared using the same procedure described for EXAMPLE 35F, substituting EXAMPLE 44B for EXAMPLE 35E. MS (MALDI) m/z 2689.0; ¹H NMR (300 MHz, CDCl₃) δ ppm 5.09-5.19 (m, 1H) 4.17-4.26 (m, 3H) 4.01-4.11 (m, 1H) 3.73-3.91 (m, 1H) 3.61-3.70 (m, 180H) 3.48-3.60 (m, 4H) 3.35-3.44 (m, 5H) 2.23-2.36 (m, 4H) 1.54-1.84 (m, 6H) 1.21-1.36 (m, 48H) 0.82-0.93 (m, 6H).

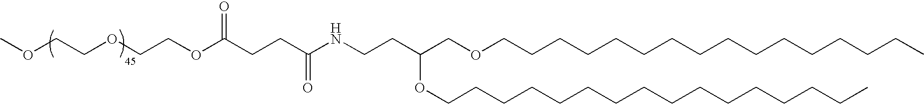

### Example 45

### 3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hexatetracontaoxanonatriacontahect-1-yl 4-{[3,4-bis(hexadecyloxy)butyl]amino}-4-oxobutanoate

EXAMPLE 37B (100 mg) and mPEG-COOH (278 mg, PSA-288, Creative PEGWorks) were combined in dichloromethane (2 mL). N¹-((ethylimino)methylene)-N³, N³-dimethylpropane-1,3-diamine hydrochloride (346 mg) was added followed by 4-(dimethylamino)pyridine (2 mg). The mixture was stirred overnight at room temperature then loaded directly onto a 4 g silica gel column (Analogix) and purified (Analogix 280, dichloromethane:methanol 0-20%). (MALDI) m/z 2628.4; ¹H NMR (300 MHz, CDCl₃) δ ppm 4.19-4.28 (m, 2H) 3.83-3.92 (m, 1H) 3.65 (none, 180H) 3.36-3.59 (m, 16H) 2.69 (t, J=6.78 Hz, 2H) 2.43 (t, J=6.95 Hz, 2H) 1.47-1.71 (m, 6H) 1.22-1.32 (m, 52H) 0.84-0.92 (m, 6H).

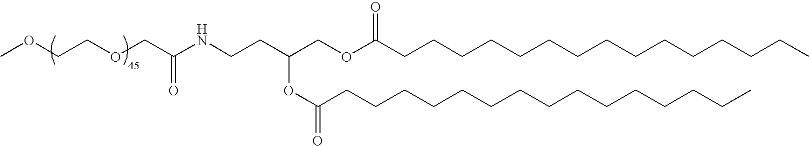

### Example 46

### 6-oxo-2-(palmitoyloxy)-8,11,14,17,20,23,26,29,32,35,38,41,44,47,50,53,56,59,62,65,68,71,74,77,80,83,86,89,92,95,98,101,104,107,110,113,116,119,122,125,128,131,134,137,140,143-hexatetracontaoxa-5-azate tratetracontahect-1-yl palmitate

This example was prepared using the same procedure described for EXAMPLE 35F, substituting EXAMPLE 44B for EXAMPLE 35E. MS (MALDI) m/z 2835.3; ¹H NMR (300 MHz, CDCl₃) δ ppm 5.07-5.20 (m, 1H) 4.24 (dd, J=11.90, 3.57 Hz, 1H) 4.06 (dd, J=11.90, 6.35 Hz, 1H) 3.98 (s, 2H) 3.61-3.68 (m, 180H) 3.49-3.60 (m, 4H) 3.36-3.48 (m, 5H) 2.25-2.36 (m, 4H) 1.77-1.87 (m, 2H) 1.26 (m, 48H) 0.83-0.93 (m, 6H).

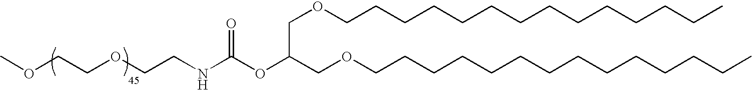

### Example 47

### 2-(tetradecyloxy)-1-((tetradecyloxy)methyl)ethyl 3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hex atetracontaoxanonatriacontahect-1-ylcarbamate

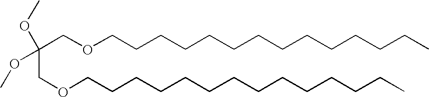

### Example 47A

### 1-(2,2-dimethoxy-3-(tetradecyloxy)propoxy)tetradecane

To a solution of 2,2-dimethoxypropane-1,3-diol (1 g) in toluene (30 mL) at 0° C. was added NaH (1.484 g). The mixture was stirred at room temperature for 1 hour. The mixture was cooled to 0° C., and 1-bromotetradecane (4.99 mL) was added. The mixture was heated at reflux for 2 hours. The mixture was cooled to 0° C., and ethanol was added until it became clear. The mixture was concentrated. The concentrate was taken up in dichloromethane and dried onto silica gel. The silica was loaded into an Analogix DASI module, and the product was isolated by flash chromatography (Analogix, SF65×200 g, 2% ethyl acetate/hexanes for six column volumes, then 4% ethyl acetate/hexanes until major product eluted). MS (ESI) m/z 512 (M-CH₃+1).

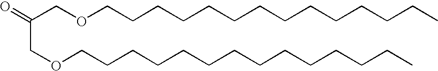

### Example 47B

### 1,3-bis(tetradecyloxy)propan-2-one

To a solution of 1-(2,2-dimethoxy-3-(tetradecyloxy)propoxy)tetradecane (2.2 g) in tetrahydrofuran (60 mL) was added 6N hydrogen chloride (5.55 mL). The mixture was stirred at room temperature overnight then concentrated. The concentrate was taken up in ethyl acetate, washed with saturated NaHCO₃, dried over Na₂SO₄, filtered, and concentrated. The concentrate was dissolved in dichloromethane and concentrated onto silica gel. The silica gel was loaded into an Analogix DASI module, and the product was isolated by flash chromatography (Analogix, SF65×200 g, 2% ethyl acetate/hexanes for six column volumes, then 4% ethyl acetate/hexanes until the product eluted. MS (ESI) m/z 500.4 (M+18).

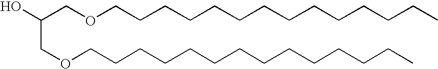

### Example 47C

### 1,3-bis(tetradecyloxy)propan-2-ol

To a solution of 1,3-bis(tetradecyloxy)propan-2-one (0.68 g) in tetrahydrofuran (13 mL) at 0° C. was added sodium borohydride (0.085 g) and water (0.867 mL). The mixture was stirred at room temperature for 1 hour, cooled to 0° C., and quenched with 1N HCl. The mixture was extracted with ethyl acetate. The extract was dried over Na₂SO₄, filtered and concentrated. The concentrate was purified by flash chromatography (1:5 ethyl acetate/hexanes). MS (ESI) m/z 484 (M+1)⁺, 502 (M+18)⁺.

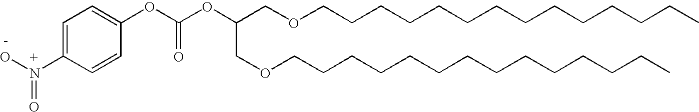

### Example 47D

### 1,3-bis(tetradecyloxy)propan-2-yl 4-nitrophenyl carbonate

To a solution of 1,3-bis(tetradecyloxy)propan-2-ol (0.3 g) in dichloromethane (3 mL) at 0° C. were added triethlyamine (0.129 mL) and 4-nitrophenyl carbonochloridate (0.137 g). The mixture was stirred at room temperature overnight and concentrated. The concentrate was purified by flash chromatography (1:10 ethyl acetate/hexanes). ¹H NMR (300 MHz, CDCl₃) δ ppm 8.24-8.30 (m, 2H), 7.37-7.42 (m, 2H), 5.06-5.13 (m, 1H), 3.67 (d, J=5.16 Hz, 4H), 3.41-3.55 (m, 4H), 1.55-1.60 (m, 4H), 1.19-1.38 (m, 44H), 0.85-0.90 (m, 6H).

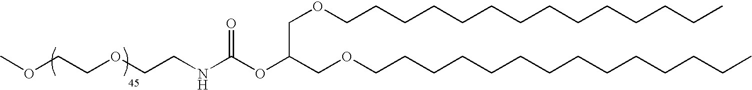

### Example 47E

### 2-(tetradecyloxy)-1-((tetradecyloxy)methyl)ethyl 3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hex atetracontaoxanonatriacontahect-1-ylcarbamate

To a solution of CH₃O-PEG2000-NH₂ (12 2000-2 Rapp Polymere, 0.2 g) in dichloromethane (1 mL) were added 1,3-bis(tetradecyloxy)propan-2-yl 4-nitrophenyl carbonate (0.195 g) and triethylamine (0.015 g). The mixture was stirred at room temperature overnight. The mixture was directly purified by flash chromatography (5-20% methanol/dichloromethane). ¹H NMR (300 MHz, CDCl₃) δ ppm 3.53-3.66 (m, 180H), 3.32-3.49 (m, 9H), 3.38 (s, 3H), 1.51-1.59 (m, 4H), 1.21-1.35 (m, 44H), 0.86-0.90 (m, 6H); MS (MALDI) m/z 2549.

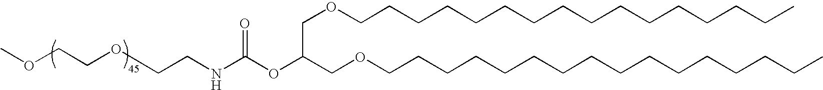

### Example 48

### 2-(hexadecyloxy)-1-((hexadecyloxy)methyl)ethyl 3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hexat etracontaoxanonatriacontahect-1-ylcarbamate

This EXAMPLE was prepared as described in EXAMPLE 47, substituting hexadecyl methanesulfonate for 1-bromotetradecane in EXAMPLE 47A. ¹H NMR (300 MHz, CDCl₃) δ ppm 3.54-3.66 (m, 180H), 3.32-3.49 (m, 9H), 3.38 (s, 3H), 1.51-1.59 (m, 4H), 1.21-1.36 (m, 48H), 0.86-0.90 (m, 6H); MS (MALDI) m/z 2614.

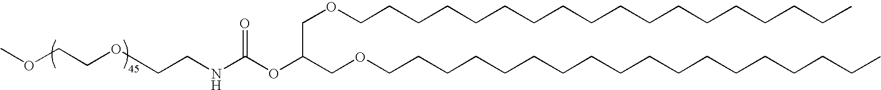

### Example 49

### 2-(octadecyloxy)-1-((octadecyloxy)methyl)ethyl 3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hexat etracontaoxanonatriacontahect-1-ylcarbamate

This EXAMPLE was prepared as described in EXAMPLE 47 substituting octadecyl methanesulfonate for 1-bromotetradecane in EXAMPLE 47A. ¹H NMR (300 MHz, CDCl₃) δ ppm 3.52-3.66 (m, 180H), 3.32-3.49 (m, 9H), 3.38 (s, 3H), 1.51-1.59 (m, 4H), 1.21-1.36 (m, 52H), 0.86-0.90 (m, 6H); MS (MALDI) m/z 2557.

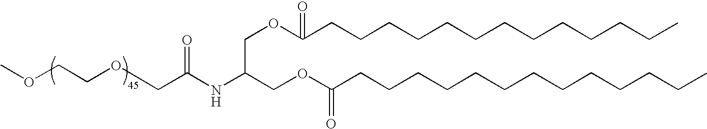

### Example 50

### 2-2,5,8,11,14,17,20,23,26,29,32,35,38,41,44,47,50,53,56,59,62,65,68,71,74,77,80,83,86,89,92,94,97,100,103,106,109,112,115,118,121,124,127,130,133,136-hexatetracontaoxaoctatriacontahectanamidopropane-1,3-diyl ditetradecanoate

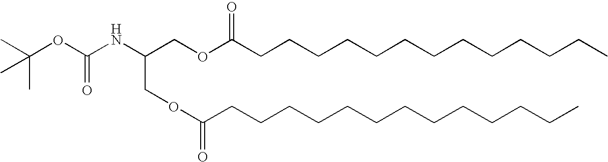

### Example 50A

### 2-(tert-butoxycarbonylamino)propane-1,3-diyl ditetradecanoate

To a solution of tetradecanoic acid (1.051 g) in dichloromethane (10 mL) at 0° C. were added tert-butyl 1,3-dihydroxypropan-2-ylcarbamate (0.40 g), 4-(dimethylamino)pyridine (0.562 g), N-methylmorpholine (1.150 mL), and 1-ethyl-3-(3-(dimethylamino)propyl)carbodiimide hydrochloride (0.882 g). The mixture was stirred at room temperature overnight. The mixture was partitioned between water and dichloromethane. The aqueous layer was extracted with dichloromethane. The extract were dried over Na₂SO₄, filtered, and concentrated. The concentrate was purified by flash chromatography (1:10 ethyl acetate/hexanes). MS (ESI) m/z 512.4 (M-CO₂-tert-butyl+1)⁺.

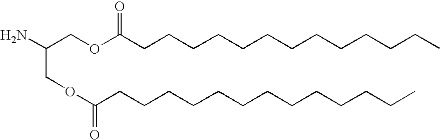

### Example 50B

### 2-aminopropane-1,3-diyl ditetradecanoate

To a solution of 2-(tert-butoxycarbonylamino)propane-1,3-diyl ditetradecanoate in dichloromethane (10 mL) was added trifluoroacetic acid. The mixture was stirred at room temperature for 2 hours then concentrated. The concentrate was purified by flash chromatography. MS (ESI) m/z 512.4 (M+1)⁺.

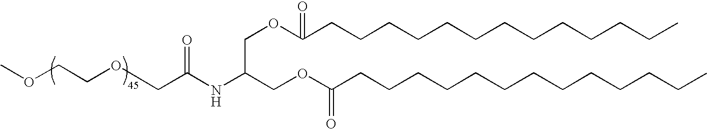

### Example 50C

### 2-2,5,8,11,14,17,20,23,26,29,32,35,38,41,44,47,50,53,56,59,62,65,68,71,74,77,80,83,86,89,92,94,97,100,103,106,109,112,115,118,121,124,127,130,133,136-hexatetracontaoxaoctatriacontahectanamidopropane-1,3-diyl ditetradecanoate

To a flask was charged with mPEG2000-SCM (Laysan, 0.2 g) and 2-aminopropane-1,3-diyl ditetradecanoate (0.077 g) was added dichloromethane (2 mL). The mixture was stirred at room temperature overnight and concentrated. The concentrate was purified by flash chromatography (5-20% methanol/dichloromethane). ¹H NMR (300 MHz, CDCl₃) δ ppm 4.11-4.21 (m, 4H), 4.01 (s, 2H), 3.53-3.68 (m, 180H), 3.39-3.42 (m, 1H), 3.38 (s, 3H), 2.31 (t, J=7.46 Hz, 4H), 1.57-1.64 (m, 4H), 1.20-1.37 (m, 40H), 0.85-0.90 (m, 6H); MS (MALDI) m/z 2632.

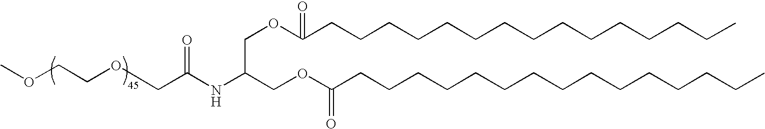

### Example 51

### 2-2,5,8,11,14,17,20,23,26,29,32,35,38,41,44,47,50,53,56,59,62,65,68,71,74,77,80,83,86,89,92,94,97,100,103,106,109,112,115,118,121,124,127,130,133,136-hexatetracontaoxaoctatriacontahectanamidopropane-1,3-diyl dipalmitate

This EXAMPLE was prepared as described in EXAMPLE 50, substituting hexadecanoic acid for tetradecanoic acid in EXAMPLE 50A. ¹H NMR (300 MHz, CDCl₃) δ ppm 4.10-4.21 (m, 4H), 4.01 (s, 2H), 3.53-3.69 (m, 180H), 3.39-3.42 (m, 1H), 3.38 (s, 3H), 2.31 (t, J=7.63 Hz, 4H), 1.56-1.63 (m, 4H), 1.20-1.33 (m, 44H), 0.85-0.91 (m, 6H); MS (MALDI) m/z 2732.

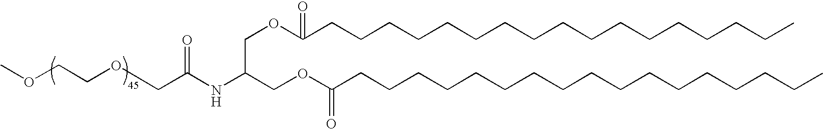

### Example 52

### 2-2,5,8,11,14,17,20,23,26,29,32,35,38,41,44,47,50,53,56,59,62,65,68,71,74,77,80,83,86,89,92,94,97,100,103,106,109,112,115,118,121,124,127,130,133,136-hexatetracontaoxaoctatriacontahectanamidopropane-1,3-diyl distearate

This EXAMPLE was prepared as in EXAMPLE 50, substituting octadecanoic acid for tetradecanoic acid in EXAMPLE 50A. ¹H NMR (300 MHz, CDCl₃) δ ppm 4.10-4.21 (m, 4H), 4.01 (s, 2H), 3.53-3.69 (m, 180H), 3.39-3.42 (m, 1H), 3.38 (s, 3H), 2.31 (t, J=7.63 Hz, 4H), 1.57-1.63 (m, 4H), 1.21-1.33 (m, 48H), 0.85-0.90 (m, 6H); MS (MALDI) m/z 2832.

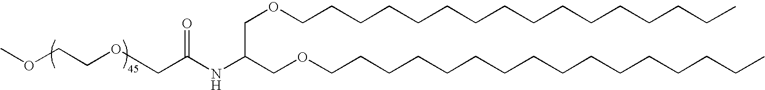

### Example 53

### N-(2-(hexadecyloxy)-1-((hexadecyloxy)methyl)ethyl)-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-h exatetracontaoxanonatriacontahectan-139-amide

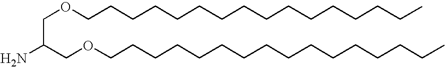

### Example 53A

In a 100 mL round-bottomed flask was added N-Boc-serinol (1,1-dimethylethyl (2-hydroxy-1-(hydroxymethyl)ethyl)carbamate) (2.0 g) and sodium hydride (1.255 g) in N,N-dimethylformamide (50 mL). The mixture was cooled using an ice/water bath, and 1-bromohexadecane (7.98 g) was added to it. The mixture was heated at 70° C. overnight, then cooled to room temperature. The mixture was cooled to 0° C. and quenched with a few drops of cold water. The mixture was diluted with saturated ammonium chloride (50 mL). The aqueous layer was extracted with ethyl acetate, and the extract was washed with brine, dried over Na₂SO₄, and concentrated. The concentrate was added to a silica gel column and was eluted with ethyl acetate/hexane (1:9). The product, tert-butyl 1,3-bis(hexadecyloxy) propan-2-ylcarbamate, was directly used for the next step.

In a 100 mL round-bottomed flask was added tert-butyl 1,3-bis(hexadecyloxy)propan-2-ylcarbamate (5.0 g) and CH₂Cl₂ (40 mL). Trifluoroacetic acid (20 mL) was then added dropwise. The mixture was stirred under nitrogen for 3 hours and concentrated. The concentrate was added to a silica gel column and eluted with CH₂Cl₂/methanol (9:1). The product was dried under vacuum. ¹H NMR (300 MHz, CDCl₃) δ ppm 3.53-3.63 (m, 4H), 3.42-3.46 (t, 4H), 3.23 (m, 1H), 2.92-2.97 (m, 2H), 1.53-1.64 (m, 4H), 1.18-1.40 (m, 52H), 0.86-0.90 (t, 6H). MS (ESI) m/z 540.6 (M+1)⁺.

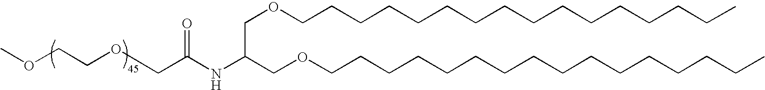

### Example 53B

Into a 40 mL glass vial was added 1,3-bis(hexadecyloxy)propan-2-amine (1.75 g) and mPEG2000-SCM (Laysan, 0.25 g, 1.081 mmol) in CH₂Cl₂ (10 mL). Triethylamine (0.50 mL) was added dropwise. The reaction solution was stirred under nitrogen for one day. The crude product was added to a silica gel column and was eluted with CH₂Cl₂/methanol (9:1). The product was dried under vacuum. ¹H NMR (300 MHz, CDCl₃) δ ppm 4.17-4.18 (m, 1H), 4.14 (s, 2H), 3.86-3.88 (m, 4H), 3.74-3.76 (t, 4H), 3.61-3.71 (m, 180H), 3.38 (s, 3H), 1.51-1.59 (m, 4H), 1.23-1.32 (m, 56H), 0.86-0.90 (m, 6H); MS (MALDI) m/z 2700.

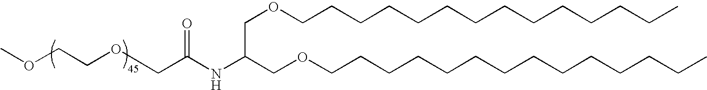

### Example 54

### N-(2-(tetradecyloxy)-1-((tetradecyloxy)methyl)ethyl)-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide

This EXAMPLE was prepared as described in EXAMPLE 53, substituting 1-bromotetradecane for 1-bromohexadecane in EXAMPLE 53A. ¹H NMR (300 MHz, CDCl₃) δ ppm 4.18 (m, 1H), 4.10 (s, 2H), 3.86-3.89 (m, 4H), 3.72-3.75 (t, 4H), 3.61-3.71 (m, 180H), 3.38 (s, 3H), 1.50-1.60 (m, 4H), 1.24-1.30 (m, 48H), 0.86-0.90 (m, 6H); MS (MALDI) m/z 2400.

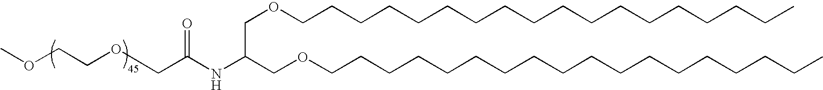

### Example 55

### N-(2-(octadecyloxy)-1-((octadecyloxy)methyl)ethyl)-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-h exatetracontaoxanonatriacontahectan-139-amide

This EXAMPLE was prepared as described in EXAMPLE 53, substituting 1-bromooctadecane hexadecane for 1-bromotetradecane in EXAMPLE 53A. ¹H NMR (300 MHz CDCl₃) δ ppm 4.14-4.20 (m, 1H), 4.08 (s, 2H), 3.86-3.89 (t, 4H), 3.71-3.75 (m, 4H), 3.61-3.70 (m, 180H), 3.38 (s, 3H), 1.50-1.56 (m, 4H), 1.20-1.30 (m, 64H), 0.86-0.90 (m, 6H); MS (MALDI) m/z 2900.

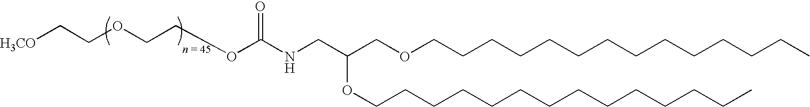

### Example 56

### N-(2,3-dimyristyloxypropyl)carbamate polyethyleneglycol-2000 methyl ether

EXAMPLE 56 was prepared using the known synthetic route; see: Heyes, J.; Hall, K.; Tailor, V.; Lenz, R.; MacLachlan, I. J. Controlled Release 2006, 112, 280-290.

We claim:

1. A cationic lipid having Formula (I) 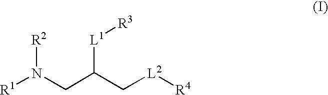
wherein
R¹ and R² are independently H, cycloalkyl, cycloalkenyl or R⁵; or
R¹ and R², taken together with the atoms to which they are attached, are heterocycloalkyl or heteroaryl;
L¹ is O, OC(O) or (O)CO;
L² is O, OC(O) or (O)CO;
one of R³ and R⁴ is H, and the other is C₁₄-C₂₀-alkenyl or C₁₄-C₂₀-alkyl; or
R³ and R⁴ are independently C₁₄-C₂₀-alkenyl or C₁₄-C₂₀-alkyl; or
R³ and R⁴ combine to form CR²⁰R²¹, wherein R²⁰ is H and R²¹ is C₁₄-C₂₀-alkenyl or C₁₄-C₂₀-alkyl; or R²⁰ and R²¹ are the same or are different and are C₁₄-C₂₀-alkenyl, C₁₄-C₂₀-alkyl, or (CH₂O)—C₁₄-C₂₀ alkenyl;
R⁵ is alkyl, which is unsubstituted or substituted with one or more R⁶, OR⁶, SR⁶, S(O)R⁶, SO₂R⁶, C(O)R⁶, CO(O)R⁶, OC(O)R⁶, OC(O)OR⁶, NH₂, NHR⁶, N(R⁶)₂, NHC(O)R⁶, NR⁶C(O)R⁶, NHS(O)₂R⁶, NR⁶S(O)₂R⁶, NHC(O)OR⁶, NR⁶C(O)OR⁶, NHC(O)NH₂, NHC(O)NHR⁶, NHC(O)N(R⁶)₂, NR⁶C(O)NHR⁶, NR⁶C(O)N(R⁶)₂, C(O)NH₂, C(O)NHR⁶, C(O)N(R⁶)₂, C(O)NHOH, C(O)NHOR⁶, C(O)NHSO₂R⁶, C(O)NR⁶SO₂R⁶, SO₂NH₂, SO₂NHR⁶, SO₂N(R⁶)₂, C(O)H, C(O)OH, C(N)NH₂, C(N)NHR⁶, C(N)N(R⁶)₂, CNOH, CNOCH₃, OH, (O), CN, N₃, NO₂, CF₃, CF₂CF₃, OCF₃, OCF₂CF₃, F, Cl, Br or I;
R⁶ is R⁷, R⁸, R⁹, or R¹⁰;
R⁷ is phenyl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;
R⁸ is heteroaryl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;
R⁹ is cycloalkyl, cycloalkenyl, heterocycloalkyl or heterocycloalkenyl, each of which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;
R¹⁰ is alkyl, alkenyl or alkynyl;
wherein each foregoing cyclic moiety is independently unsubstituted or substituted with one or more R¹¹, OR¹¹, SR¹¹, S(O)R¹¹, SO₂R¹¹, C(O)R¹¹, CO(O)R¹¹, OC(O)R¹¹, OC(O)OR¹¹, NH₂, NHR¹¹, N(R¹¹)₂, NHC(O)R¹¹, NR¹¹C(O)R¹¹, NHS(O)₂R¹¹, N R¹¹S(O)₂R¹¹, NHC(O)OR¹¹, NR¹⁰C(O)OR¹¹, NHC(O)NH₂, NHC(O)NHR¹¹, NHC(O)N(R¹¹)₂, NR¹¹C(O)NHR¹¹, NR¹¹C(O)N(R¹¹)₂, C(O)NH₂, C(O)NHR¹¹, C(O)N(R¹¹)₂, C(O)NHOH, C(O)NHOR¹¹, C(O)NHSO₂R¹¹, C(O)NR¹¹SO₂R¹¹, SO₂NH₂, SO₂NHR¹¹, SO₂N(R¹¹)₂, C(O)H, C(O)OH, C(N)NH₂, C(N)NHR¹¹, C(N)N(R¹¹)₂, CNOH, CNOCH₃, OH, (O), CN, N₃, NO₂, CF₃, CF₂CF₃, OCF₃, OCF₂CF₃, F, Cl, Br or I;
R¹¹ is R¹²R¹³, R¹⁴, or R¹⁵;
R¹² is phenyl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;
R¹³ is heteroaryl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;
R¹⁴ is cycloalkyl, cycloalkenyl, heterocycloalkyl or heterocycloalkenyl, each of which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;
R¹⁵ is alkyl, alkenyl or alkynyl, each of which is unsubstituted or substituted with one or two of independently selected R¹⁶, OR¹⁶, SR¹⁶, S(O)₂R¹⁶, C(O)OH, NH₂, NHR¹⁶N(R¹⁶)₂, C(O)R¹⁶,C(O)NH₂, C(O)NHR¹⁶, C(O)N(R¹⁶)₂, NHC(O)R¹⁶, NR¹⁶C(O)R¹⁶, NHC(O)OR¹⁶NR¹⁶C(O)OR¹⁶, OH, F, Cl, Br or I;
R¹⁶ is alkyl, alkenyl, alkynyl, or R¹⁷;
R¹⁷ is phenyl, heteroaryl, cycloalkyl, cycloalkenyl, heterocycloalkyl or heterocycloalkenyl, each of which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;
wherein R¹², R¹³, R¹⁴, and R¹⁷ are independently unsubstituted or substituted with one or more R¹⁸, OR¹⁸, SR¹⁸, S(O)R¹⁸, SO₂R¹⁸, C(O)R¹⁸, CO(O)R¹⁸, OC(O)R¹⁸, OC(O)OR¹⁸, NH₂, NHR¹⁸, N(R¹⁸)₂, NHC(O)R¹⁸, NR¹⁸C(O)R¹⁸, NHS(O)₂R¹⁸, NR¹⁸S(O)₂R¹⁸, NHC(O)OR¹⁸, NR¹⁸C(O)OR¹⁸, NHC(O)NH₂, NHC(O)NHR¹⁸, NHC(O)N(R¹⁸)₂, NR¹⁸C(O)NHR¹⁸, NR¹⁸C(O)N(R¹⁸)₂, C(O)NH₂, C(O)NHR¹⁸, C(O)N(R¹⁸)₂, C(O)NHOH, C(O)NHOR¹⁸, C(O)NHSO₂R¹⁸, C(O)NR¹⁸O₂R¹⁸, SO₂NH₂, SO₂NHR¹⁸, SO₂N(R¹⁸)₂, C(O)H, C(O)OH, C(N)NH₂, C(N)NHR¹⁸, C(N)N(R¹⁸)₂, CNOH, CNOCH₃, OH, (O), CN, N₃, NO₂, CF₃, CF₂CF₃, OCF₃, OCF₂CF₃, F, Cl, Br or I; and
R¹⁸ is alkyl, alkenyl, alkynyl, phenyl, heteroaryl, cycloalkyl, cycloalkenyl, heterocycloalkyl or heterocycloalkenyl.

2. A Cationic-Based Lipid Encapsulation System (CaBLES) comprising:
one or more (PEG)-lipid conjugates,
one or more non-cationic lipids, and
one or more cationic lipids of claim 1.

3. A Lipid-Based Particle, comprising:
one or more (PEG)-lipid conjugates,
one or more non-cationic lipids,
one or more cationic lipids of claim 1, and
one or more therapeutic agents.

4. The CaBLES of claim 2, or the Lipid-Based Particle of claim 3, wherein the cationic lipids comprise about 2 to about 60 weight/weight percent of total lipid in the particle.

5. The CaBLES of claim 2, or the Lipid-Based Particle of claim 3, wherein one or more cationic lipids are chosen from 1-(2,3-bis((9Z,12Z)-octadeca-9,12-dienyloxy)propyl)pyrrolidine, 1-(2,3-bis((9Z,12Z)-octadeca-9,12-dienyloxy)propyl)-1H-imidazole, 1-(2,3-bis((9Z,12Z)-octadeca-9,12-dienyloxy)propyl)-4-methylpiperazine, 1-(2,3-bis((9Z,12Z)-octadeca-9,12-dienyloxy)propyl)-4-methyl-1,4-diazepane, 1-(2,3-bis((9Z,12Z)-octadeca-9,12-dienyloxy)propyl)-4-phenylpiperazine, 1-(2,3-bis((9Z,12Z)-octadeca-9,12-dienyloxy)propyl)-4-pyridin-2-ylpiperazine, 1-(2,3-bis((9Z,12Z)-octadeca-9,12-dienyloxy)propyl)piperidine, 4-(2,3-bis((9Z,12Z)-octadeca-9,12-dienyloxy)propyl)morpholine, 1-((2R)-2,3-bis((9Z,12Z)-octadeca-9,12-dienyloxy)propyl)pyrrolidine, 1-((2S)-2,3-bis((9Z,12Z)-octadeca-9,12-dienyloxy)propyl)pyrrolidine, 1-(2,3-bis((9Z,12Z)-octadeca-9,12-dienyloxy)propyl)-4-ethylpiperazine, N-(2,3-bis((9Z,12Z)-octadeca-9,12-dienyloxy)propyl)-N-methyl-N-(3-(pyrrolidin-1-ylmethyl)benzyl)amine, N-(2-(4-(2,3-bis((9Z,12Z)-octadeca-9,12-dienyloxy)propyl)piperazin-1-yl)ethyl)-N,N-dimethylamine, 1-((2S)-2,3-bis((9Z,12Z)-octadeca-9,12-dienyloxy)propyl)-4-methylpiperazine, 1-((2R)-2,3-bis((9Z,12Z)-octadeca-9,12-dienyloxy)propyl)-4-methylpiperazine, 1-(2,3-bis((9Z,12Z)-octadeca-9,12-dienyloxy)propyl)-4-(2-pyrrolidin-1-ylethyl)piperazine, 2-(4-(2,3-bis((9Z,12Z)-octadeca-9,12-dienyloxy)propyl)piperazin-1-yl)pyrimidine, 1-(2,3-bis((9Z,12Z)-octadeca-9,12-dienyloxy)propyl)-N,N-diethylpyrrolidin-3-amine, 1-((9Z,12Z)-octadeca-9,12-dienyloxy)-3-pyrrolidin-1-ylpropan-2-ol, 2-[(9Z,12Z)-octadeca-9,12-dienoyloxy]-1-(pyrrolidin-1-ylmethyl)ethyl (9Z,12Z)-octadeca-9,12-dienoate, 2-[(9Z,12Z)-octadeca-9,12-dienyloxy]-1-(pyrrolidin-1-ylmethyl)ethyl (9Z,12Z)-octadeca-9,12-dienoate, 1-({2-[(8Z,11Z)-heptadeca-8,11-dienyl]-2-[(9Z,12Z)-octadeca-9,12-dienyl]-1,3-dioxolan-4-yl}methyl)pyrrolidine, 1-{2,3-bis[(5Z,8Z,11Z,14Z)-icosa-5,8,11,14-tetraenyloxy]propyl}pyrrolidine, 1-{3-[(5Z,8Z,11Z,14Z)-icosa-5,8,11,14-tetraenyloxy]-2-[(9Z,12Z)-octadeca-9,12-dienyloxy]propyl}pyrrolidine, 1-{2,3-bis[(9E,12E)-octadeca-9,12-dienyloxy]propyl}pyrrolidine, 1-{2-[(9E,12E)-octadeca-9,12-dienyloxy]-3-[(9Z,12Z)-octadeca-9,12-dienyloxy]propyl}pyrrolidine, 1-[2,3-bis(tetradecyloxy)propyl]pyrrolidine, 1-[2,3-bis(octadecyloxy)propyl]pyrrolidine, 1-{2,3-bis[(9Z)-octadec-9-enyloxy]propyl}pyrrolidine, 1-[2,3-bis(dodecyloxy)propyl]pyrrolidine, 1-{2,3-bis[(9Z,12Z)-octadeca-9,12-dienyloxy]propyl}pyrrolidin-3-ol, 1-{3-[(9Z,12Z)-hexadeca-9,12-dienyloxy]-2-[(9Z)-octadec-9-enyloxy]propyl}pyrrolidine, 1-{2,3-bis[(9Z,12Z)-octadeca-9,12-dienyloxy]propyl}-N,N-dimethylpyrrolidin-3-amine, and 1-[3-[(9Z,12Z)-hexadeca-9,12-dienyloxy]-2-(tetradecyloxy)propyl]pyrrolidine.

6. The CaBLES of claim 2, or the Lipid-Based Particle of claim 3, wherein one or more non-cationic lipids are chosen from cholesterol, cholesterol sulfate, ceramide, sphingomyelin, lecithin, sphingomyelin, egg sphingomyelin, milk sphingomyelin; egg phosphatidylcholine, hydrogenated egg phosphatidylcholine, hydrogenated soybean phosphatidylethanolamine, egg phosphatidylethanolamine, hydrogenated soybean phosphatidylcholine, soybean phosphatidylcholine, 1,2-dilauroyl-sn-glycerol, 1,2-dimyristoyl-sn-glycerol, 1,2-dipalmitoyl-sn-glycerol, 1,2-distearoyl-sn-glycerol, 1,2-dilauroyl-sn-glycero-3-phosphatidic acid, 1,2-dimyristoyl-sn-glycero-3-phosphatidic acid, 1,2-dipalmitoyl-sn-glycero-3-phosphatidic acid, 1,2-distearoyl-sn-glycero-3-phosphatidic acid, 1,2-diarachidoyl-sn-glycero-3-phosphocholine, 1,2-dilauroyl-sn-glycero-3-phosphocholine, 1,2-dimyristoyl-sn-glycero-3-phosphocholine, dioleoylphosphatidylcholine, 1,2-dierucoyl-sn-glycero-3-phosphocholine, 1-myristoyl-2-palmitoyl-sn-glycero-3-phosphocholine, 1-myristoyl-2-stearoyl-sn-glycero-3-phosphocholine, 1-palmitoyl-2-myristoyl-sn-glycero-3-phosphocholine, 1-palmitoyl-2-stearoyl-sn-glycero-3-phosphocholine, 1-stearoyl-2-myristoyl-sn-glycero-3-phosphocholine, 1-stearoyl-2-palmitoyl-sn-glycero-3-phosphocholine, 1-myristoyl-2-oleoyl-sn-glycero-3-phosphocholine, 1-palmitoyl-2-oleoyl-sn-glycero-3-phosphocholine; 1-stearoyl-2-oleoyl-sn-glycero-3-phosphocholine, 1-myristoyl-2-lyso-sn-glycero-3-phosphocholine, 1-palmitoyl-2-lyso-sn-glycero-3-phosphocholine, 1-stearoyl-2-lyso-sn-glycero-3-phosphocholine, 1,2-dipalmitoyl-sn-glycero-O-ethyl-3-phosphocholine, 1,2-dipalmitoyl-sn-glycero-3-phosphocholine; 1,2-distearoyl-sn-glycero-3-phosphocholine; 1-palmitoyl-2-linoleoyl-sn-glycero-3-phosphocholine, dioleoylphosphatidylethanolamine, palmitoyloleoyl-phosphatidylethanolamine, dioleoylphosphatidylglycerol,1,2-dilauroyl-sn-glycero-3-phosphoethanolamine, 1,2-dimyristoyl-sn-glycero-3-phosphoethanolamine, 1,2-dipalmitoyl-sn-glycero-3-phosphoethanolamine, 1,2-distearoyl-sn-glycero-3-phosphoethanolamine, 1,2-dioleoyl-sn-glycero-3-phosphoethanolamine, 1,2-dilauroyl-sn-glycero-3-phosphoglycerol, 1,2-dimyristoyl-sn-glycero-3-phosphoglycerol, 1,2-dimyristoyl-sn-glycero-3-phospho-sn-1-glycerol, 1,2-dipalmitoyl-sn-glycero-3-phosphoglycerol, 1,2-distearoyl-sn-glycero-3-phosphoglycero, 1,2-distearoyl-sn-glycero-3-phospho-sn-1-glycerol, 1-palmitoyl-2-oleoyl-sn-glycero-3-phosphoglycerol, 1-palmitoyl-2-oleoyl-sn-glycero-3-phosphoglycerol, 1,2-dipalmitoyl-sn-glycero-3-phospho-L-serine, 1,2-dimyristoyl-sn-glycero-3-phospho-L-serine, 1,2-dipalmitoyl-sn-glycero-3-phospho-L-serine, 1,2-distearoyl-sn-glycero-3-phospho-L-serine, 1,2-dioleoyl-sn-glycero-3-phospho-L-serine, or 1-palmitoyl-2-oleoyl-sn-glycero-3-phospho-L-serine.

7. The CaBLES of claim 2, or the Lipid-Based Particle of claim 3, wherein the non-cationic lipids comprise about 5 to about 90 weight/weight percent of total lipid in the particle.

8. The CaBLES of claim 2, or the Lipid-Based Particle of claim 3, wherein one or more PEG-lipid conjugates are chosen from 2-(tetradecyloxy)-1-((tetradecyloxy)methyl)ethyl 3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hexatetracontaoxanonatriacontahect-1-ylcarbamate, 2-(hexadecyloxy)-1-((hexadecyloxy)methyl)ethyl 3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hexatetracontaoxanonatriacontahect-1-ylcarbamate, 2-(octadecyloxy)-1-((octadecyloxy)methyl)ethyl 3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hexatetracontaoxanonatriacontahect-1-ylcarbamate, 2-2,5,8,11,14,17,20,23,26,29,32,35,38,41,44,47,50,53,56,59,62,65,68,71,74,77,80,83,86,89,92,94,97,100,103,106,109,112,115,118,121,124,127,130,133,136-hexatetracontaoxaoctatriacontahectanamidopropane-1,3-diyl ditetradecanoate, 2-2,5,8,11,14,17,20,23,26,29,32,35,38,41,44,47,50,53,56,59,62,65,68,71,74,77,80,83,86,89,92,94,97,100,103,106,109,112,115,118,121,124,127,130,133,136-hexatetracontaoxaoctatriacontahectanamidopropane-1,3-diyl dipalmitate, 2-2,5,8,11,14,17,20,23,26,29,32,35,38,41,44,47,50,53,56,59,62,65,68,71,74,77,80,83,86,89,92,94,97,100,103,106,109,112,115,118,121,124,127,130,133,136-hexatetracontaoxaoctatriacontahectanamidopropane-1,3-diyl distearate, N-(2-(hexadecyloxy)-1-((hexadecyloxy)methyl)ethyl)-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide, N-(2-(tetradecyloxy)-1-((tetradecyloxy)methyl)ethyl)-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide, N-(2-(octadecyloxy)-1-((octadecyloxy)methyl)ethyl)-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide, 6-oxo-2-(tetradecanoyloxy)-8,11,14,17,20,23,26,29,32,35,38,41,44,47,50,53,56,59,62,65,68,71,74,77,80,83,86,89,92,95,98,101,104,107,110,113,116,119,122,125,128,131,134,137,140,143-hexatetracontaoxa-5-azatetratetracontahect-1-yl myristate, N-[3,4-bis(tetradecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanon atriacontahectan-139-amide, N-[3,4-bis(hexadecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide, N-[3,4-bis(octadecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide, 3,7,11,15,19,23,27,31,35,39,43,47,51,55,59,63,67,71,75,79,83,87,91,95,99,103,107,111,115,119,123,127,131,135,139,143,147,151,155,159,163,167,171,175,179,182-hexatetracontaoxatrioctacontahect-1-yl 3,4-bis(tetradecyloxy)butylcarbamate, 3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hexatetracontaoxanonatriacontahect-1-yl 3,4-bis(hexadecyloxy)butylcarbamate, 3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hexatetracontaoxanonatriacontahect-1-yl 3,4-bis(octadecyloxy)butylcarbamate, N-[3,4-bis(hexadecyloxy)butyl]-N′-3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hexatetracontaoxanonatriacontahect-1-ylsuccinamide, 6-oxo-2-(tetradecanoyloxy)-7,10,13,16,19,22,25,28,31,34,37,40,43,46,49,52,55,58,61,64,67,70,73,76,79,82,85,88,91,94,97,100,103,106,109,112,115,118,121,124,127,130,133,136,139,142,145-heptatetracontaoxa-5-azahexatetracontahect-1-yl myristate, 6-oxo-2-(palmitoyloxy)-7,10,13,16,19,22,25,28,31,34,37,40,43,46,49,52,55,58,61,64,67,70,73,76,79,82,85,88,91,94,97,100,103,106,109,112,115,118,121,124,127,130,133,136,139,142,145-heptatetracontaoxa-5-azahexatetracontahect-1-yl palmitate, 3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hexatetracontaoxanonatriacontahect-1-yl 4-{[3,4-bis(hexadecyloxy)butyl]amino}-4-oxobutanoate, 6-oxo-2-(palmitoyloxy)-8,11,14,17,20,23,26,29,32,35,38,41,44,47,50,53,56,59,62,65,68,71,74,77,80,83,86,89,92,95,98,101,104,107,110,113,116,119,122,125,128,131,134,137,140,143-hexatetracontaoxa-5-azatetratetracontahect-1-yl palmitate, 1,2-distearoyl-sn-glycerol-methoxypolyethyleneglycol-750,1,2-dimyristoyl-sn-glycerol-methoxypolyethyleneglycol-750,1,2-dipalmitoyl-sn-glycerol-methoxypolyethyleneglycol-750, poly(oxy-1,2-ethanediyl)-2000-1-(3)-cholest-5-en-3-yl-omega-hydroxy, 1,2-dipalmitoyl-sn-glycerol-methoxypolyethyleneglycol-5000, poly(oxy-1,2-ethanediyl)-5000-1-(3)-cholest-5-en-3-yl-omega-hydroxy, (2S,3R,E)-3-hydroxy-2-stearamidooctadec-4-enyl polyethyleneglycol-2000 methyl ether succinate, (2S,3R,E)-3-hydroxy-2-icosanamidooctadec-4-enyl polyethyleneglycol-2000 methyl ether succinate, N-(2,3-dimyristyloxypropyl)carbamate polyethyleneglycol-2000 methyl ether, N-(carbonylmethoxypolyethyleneglycol-750)-1,2-dimyristoyl-sn-glycero-phosphatidylethanolamine, N-(carbonyl-methoxypolyethyleneglycol-750)-1,2-distearoyl-sn-glycero-3-phosphoethanolamine, N-(carbonyl-methoxypolyethyleneglycol-750)-1,2-dipalmitoyl-sn-glycero-3-phosphoethanolamine, N-(carbonyl-methoxypolyethyleneglycol-2000)-1,2-dimyristoyl-sn-glycero-3-phosphoethanolamine, N-(carbonyl-methoxypolyethyleneglycol-2000)-1,2-dipalmitoyl-sn-glycero-3-phosphoethanolamine, N-(carbonyl-methoxypolyethyleneglycol-2000)-1,2-distearoyl-sn-glycero-3-phosphoethanolamine, N-(carbonyl-methoxypolyethyleneglycol-2000)-dioleoyl-phosphatidylethanolamine, 1,2-distearoyl-sn-glycerol-methoxypolyethyleneglycol-2000,1,2-dimyristoyl-sn-glycerol-methoxypolyethyleneglycol-2000,1,2-dipalmitoyl-sn-glycerol-methoxypolyethyleneglycol-2000, mPEG-2000-cholesterol, octanoyl-mPEG-2000-ceramide, palmitoyl-mPEG-2000-ceramide, N-(carbonyl-methoxypolyethyleneglycol-5000)-1,2-dimyristoyl-sn-glycero-3-phosphoethanolamine, N-(carbonyl-methoxypolyethyleneglycol-5000)-1,2-dipalmitoyl-sn-glycero-3-phosphoethanolamine, N-(carbonyl-methoxypolyethyleneglycol-5000)-1,2-distearoyl-sn-glycero-3-phosphoethanolamine, 1,2-dimyristoyl-sn-glycerol-methoxypolyethyleneglycol-5000,1,2-distearoyl-sn-glycerol-methoxypolyethyleneglycol-5000, mPEG-5000-cholesterol, octanoyl-mPEG-5000-ceramide, or palmitoyl-mPEG-5000-ceramide.

9. The CaBLES of claim 2, or the Lipid-Based Particle of claim 3, wherein the PEG-lipid conjugates comprise 0.1 to about 20 weight/weight percent of total lipid in the particle.

10. The Lipid-Based Particle of claim 3, wherein the therapeutic agent is RNA, antisense oligonucleotide, a DNA, a plasmid, a ribosomal RNA (rRNA), a micro RNA (miRNA), transfer RNA (tRNA), a small inhibitory RNA (siRNA), small nuclear RNA (snRNA), an antigen, fragments thereof, a protein, a peptide, small-molecules, or mixtures thereof.

11. The CaBLES of claim 2 or Lipid-Based Particle of claim 3, wherein said PEG lipid conjugate is about 0.1-20 weight/weight % of total lipid in particle, said DSPC is about 1-30 weight/weight % of total lipid in particle, said cholesterol is about 5-45 weight/weight % of total lipid in particle, and said cationic lipid is about 5-60 weight/weight % of total lipid in particle.

12. A pharmaceutical composition comprising a Lipid-Based Particle of claim 3 and a pharmaceutically acceptable carrier.

13. A pharmaceutical composition of claim 12, wherein said Lipid-Based Particle comprises cholesterol, DSPC, 1-(2,3-bis((9Z,12Z)-octadeca-9,12-dienyloxy)propyl)pyrrolidine, one or more PEG-lipid conjugates, and one or more nucleic acids.

14. A pharmaceutical composition of claim 13, wherein said (PEG)-lipid conjugates are about 0.1-20 weight/weight % of total lipid in particle, said DSPC is about 1-30 weight/weight % of total lipid in particle, said cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

15. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugate is N-(2,3-dimyristyloxypropyl)carbamate polyethyleneglycol-2000 methyl ether, and said therapeutic agent is siRNA.

16. The Lipid-Based Particle of claim 15, wherein said N-(2,3-dimyristyloxypropyl)carbamate polyethyleneglycol-2000 methyl ether is about 0.1-20 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

17. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugate is N-(carbonyl-methoxypolyethyleneglycol-2000)-1,2-distearoyl-sn-glycero-3-phosphoethanolamine, and said therapeutic agent is siRNA.

18. The Lipid-Based Particle of claim 17, wherein said N-(carbonyl-methoxypolyethyleneglycol-2000)-1,2-distearoyl-sn-glycero-3-phosphoethanolamine is about 0.1-20 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

19. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugate is N-(carbonyl-methoxypolyethyleneglycol-2000)-1,2-dipalmitoyl-sn-glycero-3-phosphoethanolamine, and said therapeutic agent is siRNA.

20. The Lipid-Based Particle of claim 19, wherein said N-(carbonyl-methoxypolyethyleneglycol-2000)-1,2-dipalmitoyl-sn-glycero-3-phosphoethanolamine is about 0.1-20 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

21. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugate is N-(carbonyl-methoxypolyethyleneglycol-2000)-1,2-dimyristoyl-sn-glycero-3-phosphoethanolamine, and said therapeutic agent is siRNA.

22. The Lipid-Based Particle of claim 21, wherein said N-(carbonyl-methoxypolyethyleneglycol-2000)-1,2-dimyristoyl-sn-glycero-3-phosphoethanolamine is about 0.1-20 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

23. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugate is 1,2-distearoyl-sn-glycerol-methoxypolyethyleneglycol-2000, and said therapeutic agent is siRNA.

24. The Lipid-Based Particle of claim 23, wherein said 1,2-distearoyl-sn-glycerol methoxypolyethyleneglycol-2000 is about 0.1-20 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

25. The Lipid-Based Particle of claim 3, wherein said non-cationic lipids are cholesterol and DSPC, said cationic lipid is 1-(2,3bis((9Z,12Z)-octadeca-9,12-dienyloxy)propyl)pyrrolidine, said PEG-lipid conjugate is 1,2-dipalmitoyl-sn-glycerol-methoxypolyethyleneglycol-2000, and said therapeutic agent is siRNA.

26. The Lipid-Based Particle of claim 25, wherein said 1,2-dipalmitoyl-sn-glycerol-methoxypolyethyleneglycol-2000 is about 0.1-20 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

27. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugate is 1,2-dimyristoyl-sn-glycerol-methoxypolyethyleneglycol-2000, and said therapeutic agent is siRNA.

28. The Lipid-Based Particle of claim 27, wherein said 1,2-dimyristoyl-sn-glycerol-methoxypolyethyleneglycol-2000 is about 0.1-20 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

29. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugate is N-[3,4-bis(tetradecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide, and said therapeutic agent is siRNA.

30. The Lipid-Based Particle of claim 29, wherein said N-[3,4-bis(tetradecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide is about 0.1-20 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

31. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugate is N-[3,4-bis(hexadecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide, and said therapeutic agent is siRNA.

32. The Lipid-Based Particle of claim 31, wherein said N-[3,4-bis(hexadecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide is about 0.1-20 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

33. A pharmaceutical composition of claim 12, wherein said Lipid-Based Particle comprises, cholesterol, SPC, 1-(2,3-bis((9Z,12Z)-octadeca-9,12-dienyloxy)propyl)pyrrolidine, one or more PEG-lipid conjugates, and one or more nucleic acids.

34. A pharmaceutical composition of claim 33, wherein said (PEG)-lipid conjugates are about 0.1-20 weight/weight % of total lipid in particle, said SPC is about 1-30 weight/weight % of total lipid in particle, said cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

35. The Lipid-Based Particle of claim 3, wherein said non-cationic lipids are cholesterol and SPC, said cationic lipid is 1-(2,3-bis((9Z,12Z)-octadeca-9,12-dienyloxy)propyl)pyrrolidine, said PEG-lipid conjugate is 1,2-dipalmitoyl-sn-glycerol-methoxypolyethyleneglycol-2000 and, and said therapeutic agent is siRNA.

36. The Lipid-Based Particle of claim 35, wherein said 1,2-dipalmitoyl-sn-glycerol-methoxypolyethyleneglycol-2000 is about 0.1-20 weight/weight % of total lipid in particle, said SPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

37. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugates are 1,2-dimyristoyl-sn-glycerol-methoxypolyethyleneglycol-2000 and 1,2-dipalmitoyl-sn-glycerol-methoxypolyethyleneglycol-2000, and said therapeutic agent is siRNA.

38. The Lipid-Based Particle of claim 37, wherein said 1,2-dimyristoyl-sn-glycerol-methoxypolyethyleneglycol-2000 and 1,2-dipalmitoyl-sn-glycerol-methoxypolyethyleneglycol-2000 are about 0.1-20 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

39. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugates are 1,2-dimyristoyl-sn-glycerol-methoxypolyethyleneglycol-2000 and 1,2-distearoyl-sn-glycerol-methoxypolyethyleneglycol-2000, and said therapeutic agent is siRNA.

40. The Lipid-Based Particle of claim 39, wherein said 1,2-dimyristoyl-sn-glycerol-methoxypolyethyleneglycol-2000 and 1,2-distearoyl-sn-glycerol-methoxypolyethyleneglycol-2000 are about 0.1-20 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

41. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugates are 1,2-dipalmitoyl-sn-glycerol-methoxypolyethyleneglycol-2000 and 1,2-distearoyl-sn-glycerol-methoxypolyethyleneglycol-2000, and said therapeutic agent is siRNA.

42. The Lipid-Based Particle of claim 41, wherein said 1,2-dipalmitoyl-sn-glycerol-methoxypolyethyleneglycol-2000 and 1,2-distearoyl-sn-glycerol-methoxypolyethyleneglycol-2000 are about 0.1-20 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

43. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugates are N-(carbonyl-methoxypolyethyleneglycol-2000)-1,2-dimyristoyl-sn-glycero-3-phosphoethanolamine and N-(carbonyl-methoxypolyethyleneglycol-2000)-1,2-dipalmitoyl-sn-glycero-3-phosphoethanolamine, and said therapeutic agent is siRNA.

44. The Lipid-Based Particle of claim 43, wherein said N-(carbonyl-methoxypolyethyleneglycol-2000)-1,2-dimyristoyl-sn-glycero-3-phosphoethanolamine and N-(carbonyl-methoxypolyethyleneglycol-2000)-1,2-dipalmitoyl-sn-glycero-3-phosphoethanolamine are about 0.1-20 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

45. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugates are N-(carbonyl-methoxypolyethyleneglycol-2000)-1,2-distearoyl-sn-glycero-3-phosphoethanolamine and N-(carbonyl-methoxypolyethyleneglycol-2000)-1,2-dipalmitoyl-sn-glycero-3-phosphoethanolamine, and said therapeutic agent is siRNA.

46. The Lipid-Based Particle of claim 45, wherein said N-(carbonyl-methoxypolyethyleneglycol-2000)-1,2-distearoyl-sn-glycero-3-phosphoethanolamine and N-(carbonyl-methoxypolyethyleneglycol-2000)-1,2-dipalmitoyl-sn-glycero-3-phosphoethanolamine are about 0.1-20 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

47. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugates are N-[3,4-bis(hexadecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide and 1,2-distearoyl-sn-glycerol-methoxypolyethyleneglycol-2000, and said therapeutic agent is siRNA.

48. The Lipid-Based Particle of claim 47, wherein said N-[3,4-bis(hexadecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide and 1,2-distearoyl-sn-glycerol-methoxypolyethyleneglycol-2000 are about 0.1-20 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

49. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugates are N-(carbonyl-methoxypolyethyleneglycol-2000)-1,2-dimyristoyl-sn-glycero-3-phosphoethanolamine and N-(carbonyl-methoxypolyethyleneglycol-2000)-1,2-distearoyl-sn-glycero-3-phosphoethanolamine, and said therapeutic agent is siRNA.

50. The Lipid-Based Particle of claim 49, wherein said N-(carbonyl-methoxypolyethyleneglycol-2000)-1,2-dimyristoyl-sn-glycero-3-phosphoethanolamine and N-(carbonyl-methoxypolyethyleneglycol-2000)-1,2-distearoyl-sn-glycero-3-phosphoethanolamine are about 0.1-20 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

51. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugates are 1,2-dimyristoyl-sn-glycerol-methoxypolyethyleneglycol-2000 and N-(carbonyl-methoxypolyethyleneglycol-2000)-1,2-distearoyl-sn-glycero-3-phosphoethanolamine, and said therapeutic agent is siRNA.

52. The Lipid-Based Particle of claim 51, wherein said 1,2-dimyristoyl-sn-glycerol-methoxypolyethyleneglycol-2000 and N-(carbonyl-methoxypolyethyleneglycol-2000)-1,2-distearoyl-sn-glycero-3-phosphoethanolamine are about 0.1-20 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

53. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugates are 1,2-distearoyl-sn-glycerol-methoxypolyethyleneglycol-2000 and N-(carbonyl-methoxypolyethyleneglycol-2000)-1,2-dimyristoyl-sn-glycero-3-phosphoethanolamine, and said therapeutic agent is siRNA.

54. The Lipid-Based Particle of claim 53, wherein said 1,2-distearoyl-sn-glycerol-methoxypolyethyleneglycol-2000 and N-(carbonyl-methoxypolyethyleneglycol-2000)-1,2-dimyristoyl-sn-glycero-3-phosphoethanolamine are about 0.1-20 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

55. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugates are N-[3,4-bis(hexadecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide and N-(carbonyl-methoxypolyethyleneglycol-2000)-1,2-distearoyl-sn-glycero-3-phosphoethanolamine, and said therapeutic agent is siRNA.

56. The Lipid-Based Particle of claim 55, wherein said N-[3,4-bis(hexadecyloxy)butyl]-2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137-hexatetracontaoxanonatriacontahectan-139-amide and N-(carbonyl-methoxypolyethyleneglycol-2000)-1,2-distearoyl-sn-glycero-3-phosphoethanolamine are about 0.1-20 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

57. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugates are N-(carbonyl-methoxypolyethyleneglycol-2000)-1,2-dimyristoyl-sn-glycero-3-phosphoethanolamine and 1,2-dipalmitoyl-sn-glycerol-methoxypolyethyleneglycol-2000, and said therapeutic agent is siRNA.

58. The Lipid-Based Particle of claim 57, wherein said N-(carbonyl-methoxypolyethyleneglycol-2000)-1,2-dimyristoyl-sn-glycero-3-phosphoethanolamine and 1,2-dipalmitoyl-sn-glycerol-methoxypolyethyleneglycol-2000 are about 0.1-20 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

59. A pharmaceutical composition of claim 12, wherein said Lipid-Based Particle comprises, cholesterol, DSPC, 1-(2,3-bis((9Z,12Z)-octadeca-9,12-dienyloxy)propyl)-4-methylpiperazine, one or more PEG-lipid conjugates, and one or more nucleic acids.

60. A pharmaceutical composition of claim 59, wherein said (PEG)-lipid conjugates are about 0.1-20 weight/weight % of total lipid in particle, said DSPC is about 1-30 weight/weight % of total lipid in particle, said cholesterol is about 5-45 weight/weight % of total lipid in particle, and 1-(2,3-bis((9Z,12Z)-octadeca-9,12-dienyloxy)propyl)-4-methylpiperazine is about 5-60 weight/weight % of total lipid in particle.

61. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)-4-methylpiperazine, said PEG-lipid conjugate is N-(2,3-dimyristyloxypropyl)carbamate polyethyleneglycol-2000 methyl ether, and said therapeutic agent is siRNA.

62. The Lipid-Based Particle of claim 61, wherein said N-(2,3-dimyristyloxypropyl)carbamate polyethyleneglycol-2000 methyl ether is about 0.1-20 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)-4-methylpiperazine is about 5-60 weight/weight % of total lipid in particle.

63. A pharmaceutical composition of claim 12, wherein said Lipid-Based Particle comprises, cholesterol, DSPC, 1-((2S)-2,3-bis((9Z,12Z)-octadeca-9,12-dienyloxy)propyl)pyrrolidine, one or more PEG-lipid conjugates, and one or more nucleic acids.

64. A pharmaceutical composition of claim 63, wherein said (PEG)-lipid conjugates are about 0.1-20 weight/weight % of total lipid in particle, said DSPC is about 1-30 weight/weight % of total lipid in particle, said cholesterol is about 5-45 weight/weight % of total lipid in particle, and 1-((2S)-2,3-bis((9Z,12Z)-octadeca-9,12-dienyloxy)propyl)pyrrolidine is about 5-60 weight/weight % of total lipid in particle.

65. The Lipid-Based Particle of claim 3, wherein said non-cationic lipids are cholesterol and DSPC, said cationic lipid is 1-((2S)-2,3-bis((9Z,12Z)-octadeca-9,12-dienyloxy)propyl)pyrrolidine, said PEG-lipid conjugate is N-(2,3-dimyristyloxypropyl)carbamate polyethyleneglycol-2000 methyl ether, and said therapeutic agent is siRNA.

66. The Lipid-Based Particle of claim 65, wherein said N-(2,3-dimyristyloxypropyl)carbamate polyethyleneglycol-2000 methyl ether is about 0.1-20 weight/weight % of total lipid in particle, said DSPC is about 1-30 weight/weight % of total lipid in particle, said cholesterol is about 5-45 weight/weight % of total lipid in particle, and said 1-((2S)-2,3-bis((9Z,12Z)-octadeca-9,12-dienyloxy)propyl)pyrrolidine is about 5-60 weight/weight % of total lipid in particle.

67. A pharmaceutical composition of claim 12, wherein said Lipid-Based Particle comprises, cholesterol, DSPC, 1-((2R)-2,3-bis((9Z,12Z)-octadeca-9,12-dienyloxy)propyl)pyrrolidine, one or more PEG-lipid conjugates, and one or more nucleic acids.

68. A pharmaceutical composition of claim 67, wherein said (PEG)-lipid conjugates are about 0.1-20 weight/weight % of total lipid in particle, said DSPC is about 1-30 weight/weight % of total lipid in particle, said cholesterol is about 5-45 weight/weight % of total lipid in particle, and 1-((2R)-2,3-bis((9Z,12Z)-octadeca-9,12-dienyloxy)propyl)pyrrolidine is about 5-60 weight/weight % of total lipid in particle.

69. The Lipid-Based Particle of claim 3, wherein said non-cationic lipids are cholesterol and DSPC, said cationic lipid is 1-((2R)-2,3-bis((9Z,12Z)-octadeca-9,12-dienyloxy)propyl)pyrrolidine, said PEG-lipid conjugate is N-(2,3-dimyristyloxypropyl)carbamate polyethyleneglycol-2000 methyl ether, and said therapeutic agent is siRNA.

70. The Lipid-Based Particle of claim 69, wherein said N-(2,3-dimyristyloxypropyl)carbamate polyethyleneglycol-2000 methyl ether is about 0.1-20 weight/weight % of total lipid in particle, said DSPC is about 1-30 weight/weight % of total lipid in particle, said cholesterol is about 5-45 weight/weight % of total lipid in particle, and said 1-((2R)-2,3-bis((9Z,12Z)-octadeca-9,12-dienyloxy)propyl)pyrrolidine is about 5-60 weight/weight % of total lipid in particle.

71. A pharmaceutical composition of claim 12, wherein said Lipid-Based Particle comprises, cholesterol, DSPC, 1-{2-[(9E,12E)-octadeca-9,12-dienyloxy]-3-[(9Z,12Z)-octadeca-9,12-dienyloxy]propyl}pyrrolidine, one or more PEG-lipid conjugates, and one or more nucleic acids.

72. A pharmaceutical composition of claim 71, wherein said (PEG)-lipid conjugates are about 0.1-20 weight/weight % of total lipid in particle, said DSPC is about 1-30 weight/weight % of total lipid in particle, said cholesterol is about 5-45 weight/weight % of total lipid in particle, and 1-{2-[(9E,12E)-octadeca-9,12-dienyloxy]-3-[(9Z,12Z)-octadeca-9,12-dienyloxy]propyl}pyrrolidine is about 5-60 weight/weight % of total lipid in particle.

73. The Lipid-Based Particle of claim 3, wherein said non-cationic lipids are cholesterol and DSPC, said cationic lipid is 1-{2-[(9E,12E)-octadeca-9,12-dienyloxy]-3-[(9Z,12Z)-octadeca-9,12-dienyloxy]propyl}pyrrolidine, said PEG-lipid conjugate is 1,2-dipalmitoyl-sn-glycerol-methoxypolyethyleneglycol-2000, and said therapeutic agent is siRNA.

74. The Lipid-Based Particle of claim 73, wherein said 1,2-dipalmitoyl-sn-glycerol-methoxypolyethyleneglycol-2000 is about 0.1-20 weight/weight % of total lipid in particle, said DSPC is about 1-30 weight/weight % of total lipid in particle, said cholesterol is about 5-45 weight/weight % of total lipid in particle, and said 1-{2-[(9E,12E)-octadeca-9,12-dienyloxy]-3-[(9Z,12Z)-octadeca-9,12-dienyloxy]propyl}pyrrolidine is about 5-60 weight/weight % of total lipid in particle.

75. A method of making the Lipid-Based Particle of claim 3, comprising:
(a) mixing the cationic lipid(s), the non-cationic lipid(s) and the PEG-lipid conjugate(s);
(b) adding the mixture of step (a) to one or more therapeutic agents; and
(c) separating and purifying resulting suspension of step (b).

76. The method of claim 25, wherein said therapeutic agent is warmed to about 60° C. prior to the addition of said mixture of step (a) via needle injection.

77. The CaBLES of claim 2 which effectively encapsulate therapeutic agents, with efficiencies from about 50-100%.

78. The CaBLES of claim 2 which effectively encapsulate therapeutic agents, with efficiencies from about 80-100%.

79. The CaBLES of claim 2 used to deliver a therapeutic agent wherein one or more cationic lipids are chosen from 1-(2,3-bis((9Z,12Z)-octadeca-9,12-dienyloxy)propyl)pyrrolidine, 1-(2,3-bis((9Z,12Z)-octadeca-9,12-dienyloxy)propyl)-1H-imidazole, 1-(2,3-bis((9Z,12Z)-octadeca-9,12-dienyloxy)propyl)-4-methylpiperazine, 1-(2,3-bis((9Z,12Z)-octadeca-9,12-dienyloxy)propyl)-4-methyl-1,4-diazepane, 1-(2,3-bis((9Z,12Z)-octadeca-9,12-dienyloxy)propyl)-4-phenylpiperazine, 1-(2,3-bis((9Z,12Z)-octadeca-9,12-dienyloxy)propyl)-4-pyridin-2-ylpiperazine, 1-(2,3-bis((9Z,12Z)-octadeca-9,12-dienyloxy)propyl)piperidine, 4-(2,3-bis((9Z,12Z)-octadeca-9,12-dienyloxy)propyl)morpholine, 1-((2R)-2,3-bis((9Z,12Z)-octadeca-9,12-dienyloxy)propyl)pyrrolidine, 1-((2S)-2,3-bis((9Z,12Z)-octadeca-9,12-dienyloxy)propyl)pyrrolidine, 1-(2,3-bis((9Z,12Z)-octadeca-9,12-dienyloxy)propyl)-4-ethylpiperazine, N-(2,3-bis((9Z,12Z)-octadeca-9,12-dienyloxy)propyl)-N-methyl-N-(3-(pyrrolidin-1-ylmethyl)benzyl)amine, N-(2-(4-(2,3-bis((9Z,12Z)-octadeca-9,12-dienyloxy)propyl)piperazin-1-yl)ethyl)-N,N-dimethylamine, 1-((2S)-2,3-bis((9Z,12Z)-octadeca-9,12-dienyloxy)propyl)-4-methylpiperazine, 1-((2R)-2,3-bis((9Z,12Z)-octadeca-9,12-dienyloxy)propyl)-4-methylpiperazine, 1-(2,3-bis((9Z,12Z)-octadeca-9,12-dienyloxy)propyl)-4-(2-pyrrolidin-1-ylethyl)piperazine, 2-(4-(2,3-bis((9Z,12Z)-octadeca-9,12-dienyloxy)propyl)piperazin-1-yl)pyrimidine, 1-(2,3-bis((9Z,12Z)-octadeca-9,12-dienyloxy)propyl)-N,N-diethylpyrrolidin-3-amine, 1-((9Z,12Z)-octadeca-9,12-dienyloxy)-3-pyrrolidin-1-ylpropan-2-ol, 2-[(9Z,12Z)-octadeca-9,12-dienoyloxy]-1-(pyrrolidin-1-ylmethyl)ethyl (9Z,12Z)-octadeca-9,12-dienoate, 2-[(9Z,12Z)-octadeca-9,12-dienyloxy]-1-(pyrrolidin-1-ylmethyl)ethyl (9Z,12Z)-octadeca-9,12-dienoate, 1-({2-[(8Z,11Z)-heptadeca-8,11-dienyl]-2-[(9Z,12Z)-octadeca-9,12-dienyl]-1,3-dioxolan-4-yl}methyl)pyrrolidine, 1-{2,3-bis[(5Z,8Z,11Z,14Z)-icosa-5,8,11,14-tetraenyloxy]propyl}pyrrolidine, 1-{3-[(5Z,8Z,11Z,14Z)-icosa-5,8,11,14-tetraenyloxy]-2-[(9Z,12Z)-octadeca-9,12-dienyloxy]propyl}pyrrolidine, 1-{2,3-bis[(9E,12E)-octadeca-9,12-dienyloxy]propyl}pyrrolidine, 1-{2-[(9E,12E)-octadeca-9,12-dienyloxy]-3-[(9Z,12Z)-octadeca-9,12-dienyloxy]propyl}pyrrolidine, 1-[2,3-bis(tetradecyloxy)propyl]pyrrolidine, 1-[2,3-bis(octadecyloxy)propyl]pyrrolidine, 1-{2,3-bis[(9Z)-octadec-9-enyloxy]propyl}pyrrolidine, 1-[2,3-bis(dodecyloxy)propyl]pyrrolidine, 1-{2,3-bis[(9Z,12Z)-octadeca-9,12-dienyloxy]propyl}pyrrolidin-3-ol, 1-{2,3-bis[(9Z,12Z)-octadeca-9,12-dienyloxy]propyl}-N,N-dimethylpyrrolidin-3-amine, 1-{3-[(9Z,12Z)-hexadeca-9,12-dienyloxy]-2-[(9Z)-octadec-9-enyloxy]propyl}pyrrolidine, and 1-[3-[(9Z,12Z)-hexadeca-9,12-dienyloxy]-2-(tetradecyloxy)propyl]pyrrolidine.

80. The CaBLES of claim 2 used to deliver a therapeutic agent wherein one or more cationic lipids are chosen from 1-(2,3-bis((9Z,12Z)-octadeca-9,12-dienyloxy)propyl)pyrrolidine, 1-(2,3-bis((9Z,12Z)-octadeca-9,12-dienyloxy)propyl)-4-methylpiperazine, N-(2-(4-(2,3-bis((9Z,12Z)-octadeca-9,12-dienyloxy)propyl)piperazin-1-yl)ethyl)-N,N-dimethylamine, 1-((2S)-2,3-bis((9Z,12Z)-octadeca-9,12-dienyloxy)propyl)-4-methylpiperazine, 1-((2R)-2,3-bis((9Z,12Z)-octadeca-9,12-dienyloxy)propyl)-4-methylpiperazine, 1-(2,3-bis((9Z,12Z)-octadeca-9,12-dienyloxy)propyl)-4-(2-pyrrolidin-1-ylethyl)piperazine, 1-{2,3-bis[(9Z,12Z)-octadeca-9,12-dienyloxy]propyl}-N,N-dimethylpyrrolidin-3-amine, 1-{2,3-bis[(9E,12E)-octadeca-9,12-dienyloxy]propyl}pyrrolidine, 1-{2-[(9E,12E)-octadeca-9,12-dienyloxy]-3-[(9Z,12Z)-octadeca-9,12-dienyloxy]propyl}pyrrolidine, 1-{2,3-bis[(9Z)-octadec-9-enyloxy]propyl}pyrrolidine, 1-{2,3-bis[(9Z,12Z)-octadeca-9,12-dienyloxy]propyl}pyrrolidin-3-ol, and 1-{3-[(9Z,12Z)-hexadeca-9,12-dienyloxy]-2-[(9Z)-octadec-9-enyloxy]propyl}pyrrolidine.

81. The CaBLES of claim 2 used to deliver a therapeutic agent wherein one or more cationic lipids are chosen from 1-(2,3-bis((9Z,12Z)-octadeca-9,12-dienyloxy)propyl)pyrrolidine, 1-(2,3-bis((9Z,12Z)-octadeca-9,12-dienyloxy)propyl)-4-methylpiperazine, 1-((2R)-2,3-bis((9Z,12Z)-octadeca-9,12-dienyloxy)propyl)pyrrolidine, 1-((2S)-2,3-bis((9Z,12Z)-octadeca-9,12-dienyloxy)propyl)pyrrolidine, and 1-{2-[(9E,12E)-octadeca-9,12-dienyloxy]-3-[(9Z,12Z)-octadeca-9,12-dienyloxy]propyl}pyrrolidine.

82. The Lipid-Based Particle of claim 3, wherein the ratio of one or more (PEG)-lipid conjugates, one or more non-cationic lipids, and one or more cationic lipids of claim 1, to one or more therapeutic agents is between about 50:1 to about 5:1.

83. The Lipid-Based Particle of claim 3, wherein the ratio of one or more (PEG)-lipid conjugates, one or more non-cationic lipids, and one or more cationic lipids of claim 1, to one or more therapeutic agents is between about 30:1 to about 10:1.


SEQUENCE LISTING (2 sequences)

SEQ ID NO: 1 - DNA, 21 bp, Artificial Sequence
  siRNA sequence
ggggaaagcuggcaagauuuu

SEQ ID NO: 2 - DNA, 21 bp, Artificial Sequence
  siRNA sequence
aaucuugccagcuuuccccuu